SEQUENCE LISTING (29 sequences)

SEQ ID NO: 1 - DNA, 49 bp, synthetic construct
  misc_feature 1..49: note = Synthetic construct
  misc_feature 1..49: note = Gene32FAM-fw
  misc_feature 2: note = 6-FAMN
  source 1..49: mol_type = other DNA
catggtgattacgattcttgcccagtatgtcaatacatcagtaaaaata

SEQ ID NO: 2 - DNA, 48 bp, synthetic construct
  misc_feature 1..48: note = Synthetic construct
  misc_feature 1..48: note = Gene32-rv
  misc_feature 1: note = p 5phos
  source 1..48: mol_type = other DNA
atttttactgatgtattgacatactgggcaagaatcgtaatcaccatg

SEQ ID NO: 3 - DNA, 64 bp, synthetic construct
  misc_feature 1..64: note = Synthetic construct
  misc_feature 1: note = 5Phos
  misc_feature 32..33: note = ideoxyu
  source 1..64: mol_type = other DNA
gatcggaagagcacacgtctgaactccagtcacactctttccctacacgacgctcttccgatct

SEQ ID NO: 4 - DNA, 38 bp, synthetic construct
  misc_feature 1..38: note = Synthetic construct
  source 1..38: mol_type = other DNA
ctttaagaaggagatataccatgggcagcagccaccac

SEQ ID NO: 5 - DNA, 38 bp, synthetic construct
  misc_feature 1..38: note = Synthetic construct
  source 1..38: mol_type = other DNA
ttttttgctaatcgtctcattcctggcgcgcctatagc

SEQ ID NO: 6 - DNA, 23 bp, synthetic construct
  misc_feature 1..23: note = Synthetic construct
  source 1..23: mol_type = other DNA
atgagacgattagcaaaaaattc

SEQ ID NO: 7 - DNA, 26 bp, synthetic construct
  misc_feature 1..26: note = Synthetic construct
  source 1..26: mol_type = other DNA
ggtatatctccttcttaaagttaaac

SEQ ID NO: 8 - DNA, 38 bp, synthetic construct; identical to SEQ ID NO: 4
  misc_feature 1..38: note = Synthetic construct
  source 1..38: mol_type = other DNA

SEQ ID NO: 9 - DNA, 39 bp, synthetic construct
  misc_feature 1..39: note = Synthetic construct
  source 1..39: mol_type = other DNA
ttttttgctaatcgtctcatggaaccagagccagaaccg

SEQ ID NO: 10 - DNA, 7155 bp, synthetic construct
  misc_feature 1..7155: note = Synthetic construct
  misc_feature 1..7155: note = plasmid pHS-BspQI constructed by in-frame fusion of 6xHis-SNAPPCR DNA fragment (nts 5071-5670) to a BspQI coding gene (nts5671-6963) in pET28 His-tag-AdK-SUMO-BspQI E. coli expressionvector; no GSGSGS linker between SNAP-tag and BspQI
  source 1..7155: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccaccaccatcatcaccatggcgacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggacctgcagctataggcgcgccaggaatgagacgattagcaaaaaattcacggaacgacagttatttaagtaatagggattaccaggaaatcgtgagggaaaataccactacaatatcgtttcccttaaaagaaaaacatactctgactttaacgaaaaaaatagggctaaatcagactgctggattcggaggatggtttttccctgattcaccatgtttattaacagtaactgtactatcctctttcggtacaaaggtaacttctaaaacctttagcctttctaaagattggaatcgtgttgggcttgcttggattaacgagcattcgagtgacaccatgagcattgtcctagagtttagtgatgtggaaatagttcatacatggggacttacatgtgatgtttttaatgtccatgaattaattattgatgctatagaagatcaaaataaactaatagacgtgctaaatcaagaacatttatctcctgaaacatattatttaaaccatgactctgatactgatttaattgagaatttggaatctacagaagagataaagatagttaaccaaagccaaaagcaaatctctttaaaaaaatgctgttattgtcaacgttatatgcctgtgaacatattagttcgttcaaattcatcatttcataaacacaagagtaagaaaactggttttcaaaatgaatgtcgggcttgtaagaagtggagaataaataattcattcaatccagtcagaacaaaagaccaactacatgaatcagcagttattacacgtgaaaaaaaaatattacttaaagaacctgaaatattacagaaaatcaaaaatagaaataacggtgagggcttaaaaagtattatatggaaaaaatttgataaaaaatgctttaattgtgaaaaagaattaaccattgaagaggtacgcctagaccatacaagaccacttgcttatctgtggcctatcgatgaacacgcaacttgtttatgtgaaaaatgcaacaatacaaaacatgatatgtttcctatcgatttttatcaaggggacgaagacaaattaagacgtttagctagaattacggggttagattatgaatctctagttaagagggacgtaaatgaagttgaacttgcaagaataatcaataacattgaagactttgcaactaatgtagaggcacgtacttttcgctcaataagaaataaagtaaaagaagtacgtcccgatactgacctatttgaaattcttaaatctaaaaatattaatttatataatgaacttcaatatgaacttcttacccgtaaggattaacgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 11 - DNA, 7197 bp, synthetic construct
  misc_feature 1..7197: note = Synthetic construct
  misc_feature 1..7197: note = plasmid pHS-GSx6-BspQI constructed by in-frame fusion of6xHis-SNAP26m DNA fragment (nts 5071-5670) with a 6xGly-Serlinker (nts 5671-5712) to a BspQI coding gene (nts 5713-7005) ina pET28 14xHis-adk-sumo-BspQI E. coli expression vector
  source 1..7197: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccaccaccatcatcaccatggcgacaaagattgcgaaatgaaacgtaccaccctggatagcccgctgggcaaactggaactgagcggctgcgaacagggcctgcatgaaattaaactgctgggtaaaggcaccagcgcggccgatgcggttgaagttccggccccggccgccgtgctgggtggtccggaaccgctgatgcaggcgaccgcgtggctgaacgcgtattttcatcagccggaagcgattgaagaatttccggttccggcgctgcatcatccggtgtttcagcaggagagctttacccgtcaggtgctgtggaaactgctgaaagtggttaaatttggcgaagtgattagctatcagcagctggcggccctggcgggtaatccggcggccaccgccgccgttaaaaccgcgctgagcggtaacccggtgccgattctgattccgtgccatcgtgtggttagctctagcggtgcggttggcggttatgaaggtggtctggcggtgaaagagtggctgctggcccatgaaggtcatcgtctgggtaaaccgggtctgggacctgcagctataggcgcgccaggatccggttctggctctcctgcaggcggttctggctctggttccatgagacgattagcaaaaaattcacggaacgacagttatttaagtaatagggattaccaggaaatcgtgagggaaaataccactacaatatcgtttcccttaaaagaaaaacatactctgactttaacgaaaaaaatagggctaaatcagactgctggattcggaggatggtttttccctgattcaccatgtttattaacagtaactgtactatcctctttcggtacaaaggtaacttctaaaacctttagcctttctaaagattggaatcgtgttgggcttgcttggattaacgagcattcgagtgacaccatgagcattgtcctagagtttagtgatgtggaaatagttcatacatggggacttacatgtgatgtttttaatgtccatgaattaattattgatgctatagaagatcaaaataaactaatagacgtgctaaatcaagaacatttatctcctgaaacatattatttaaaccatgactctgatactgatttaattgagaatttggaatctacagaagagataaagatagttaaccaaagccaaaagcaaatctctttaaaaaaatgctgttattgtcaacgttatatgcctgtgaacatattagttcgttcaaattcatcatttcataaacacaagagtaagaaaactggttttcaaaatgaatgtcgggcttgtaagaagtggagaataaataattcattcaatccagtcagaacaaaagaccaactacatgaatcagcagttattacacgtgaaaaaaaaatattacttaaagaacctgaaatattacagaaaatcaaaaatagaaataacggtgagggcttaaaaagtattatatggaaaaaatttgataaaaaatgctttaattgtgaaaaagaattaaccattgaagaggtacgcctagaccatacaagaccacttgcttatctgtggcctatcgatgaacacgcaacttgtttatgtgaaaaatgcaacaatacaaaacatgatatgtttcctatcgatttttatcaaggggacgaagacaaattaagacgtttagctagaattacggggttagattatgaatctctagttaagagggacgtaaatgaagttgaacttgcaagaataatcaataacattgaagactttgcaactaatgtagaggcacgtacttttcgctcaataagaaataaagtaaaagaagtacgtcccgatactgacctatttgaaattcttaaatctaaaaatattaatttatataatgaacttcaatatgaacttcttacccgtaaggattaacgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 12 - DNA, 17 bp, synthetic construct
  misc_feature 1..17: note = Synthetic construct
  source 1..17: mol_type = other DNA
aaaaaaaaaagaagagc

SEQ ID NO: 13 - DNA, 60 bp, synthetic construct
  misc_feature 1..60: note = Synthetic construct
  misc_feature 1..60: note = BspQI Substrate FAM Forward
  misc_feature 1: note = 56FAM
  misc_feature 33..39: note = BspQI recognition sequence
  source 1..60: mol_type = other DNA
tgccgctttctgcatcagcacatcatcttcaggctcttcgtcagcctcgcgccggttcag

SEQ ID NO: 14 - DNA, 60 bp, synthetic construct; identical to SEQ ID NO: 13
  misc_feature 1..60: note = Synthetic consstruct
  misc_feature 1..60: note = BspQI Substrate Forward
  misc_feature 33..39: note = BspQI recognition sequence
  source 1..60: mol_type = other DNA

SEQ ID NO: 15 - DNA, 60 bp, synthetic construct
  misc_feature 1..60: note = Synthetic construct
  misc_feature 1..60: note = BspQI Substrate Reverse
  source 1..60: mol_type = other DNA
ctgaaccggcgcgaggctgacgaagagcctgaagatgatgtgctgatgcagaaagcggca

SEQ ID NO: 16 - DNA, 74 bp, synthetic construct
  misc_feature 1..74: note = Synthetic construct
  misc_feature 1..74: note = SNAP-FCE-T7 RNAP
  source 1..74: mol_type = other DNA
atcatcatggcgcgggcaccaaaatcgaagaagacaaggactgcgaaatgaaacgtaccaccctggatagcccg

SEQ ID NO: 17 - DNA, 38 bp, synthetic construct
  misc_feature 1..38: note = Synthetic construct
  misc_feature 1..38: note = SNAP-FCE-T7 RNAP
  source 1..38: mol_type = other DNA
caacgctgcagacgcttcgcgcccgcggtcgcgccgcc

SEQ ID NO: 18 - DNA, 20 bp, synthetic construct
  misc_feature 1..20: note = Synthetic construct
  misc_feature 1..20: note = Amplification primer
  source 1..20: mol_type = other DNA
gcgaagcgtctgcagcgttg

SEQ ID NO: 19 - DNA, 18 bp, synthetic construct
  misc_feature 1..18: note = Synthetic construct
  misc_feature 1..18: note = Amplification primer
  source 1..18: mol_type = other DNA
ggtgcccgcgccatgatg

SEQ ID NO: 20 - RNA, 25 nt, synthetic construct
  misc_feature 1..25: note = Synthetic construct
  misc_feature 1..25: note = Synthetic 25mer capping substrate
  source 1..25: mol_type = other RNA
gtagaacttcgtcgagtacgctcaa

SEQ ID NO: 21 - RNA, 173 nt, synthetic construct
  misc_feature 1..173: note = Synthetic construct
  misc_feature 1..173: note = pRNA21 transcript sequence
  source 1..173: mol_type = other RNA
gggaaataagagagaaaagaagagtaagaagaaatataagagccaccatggttaacgctgccttctgcggggcttgccttctggccatgcccttcttctctcccttgcacctgtacctcttggtctttgaataaagcctgagtaggaagaaaaaaaaaaaaaaaaaaaaaaaa

SEQ ID NO: 22 - protein, 1033 aa, synthetic construct
  REGION 1..1033: note = Synthetic construct
  REGION 1..1033: note = MISC_FEATURE - SNAP-T7-RNAP
MKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQL

SEQ ID NO: 23 - protein, 1079 aa, synthetic construct
  REGION 1..1079: note = Synthetic construct
  REGION 1..1079: note = MISC_FEATURE - SNAP-FCE
MKIEEDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGSATASAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGASATSHHHHHHHHST

SEQ ID NO: 24 - protein, 544 aa, synthetic construct
  REGION 1..544: note = Synthetic construct
  REGION 1..544: note = MISC_FEATURE - SNAP-vaccinia cap 2'-O-methyltransferase
HHHHHHGDKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLGPAAIGAPGSGSGSPAGGSGSGSDVVSLDKPFMYFEEIDNELDYEPESANEVAKKLPYQGQLKLLLGELFFLSKLQRHGILDGATVVYIGSAPGTHIRYLRDHFYNLGVIIKWMLIDGRHHDPILNGLRDVTLVTRFVDEEYLRSIKKQLHPSKIILISDVRSKRGGNEPSTADLLSNYALQNVMISILNPVASSLKWRCPFPDQWIKDFYIPHGNKMLQPFAPSYSAEMRLLSIYTGENMRLTRVTKSDAVNYEKKMYYLNKIVRNKVVVNFDYPNQEYDYFHMYFMLRTVYCNKTFPTTKAKVLFLQQSIFRFLNIPTTSTEKVSHEPIQRKISSKNSMSKNRNSKRSVRSNKLE

SEQ ID NO: 25 - protein, 1968 aa, synthetic construct
  REGION 1..1968: note = Synthetic construct
  REGION 1..1968: note = MISC_FEATURE - SNAP-FCE-T7-RNAP
MKIEEHHHHHHHHGAGTKIEEDKDCEMKRTTLDSPLGKLELSGCEQGLHRIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGGATAGAKRLQRCQDVNQVCEIYNSKGGIGELELRFDKLPQNLFAGVFDKLKPDGEIQTTMRVSNRDGVAREITFGGGVKTNEIFVKKQNICVFDVVDIFSYKVAVSTEETVVEKPTMETTAGVRFKIRLSVEDVVKDWRIDLTAVKTAELGKIAQHTASIVQRTFPDNLLKLTGAEVAKLAADSYELELEYTGKSPATNEKVNVAAKYAVELLSSVRNANSTAAASFGESVSDLCRVAKIIHTHEYANVVCRTPSFKMLLPQVVSLTKSSYYGGLYPPENLWLAGKTDGVRALVVCEDGVAKVITAESVDITHGVCSATTILDCELNVDAKILYVFDVIISNNTQVYTQPFSTRITTDISDIKIDGYKIEMKPFVKVVKADEATFKSAYKAPHNEGLIMIEDGAAYAATKTYKWKPLSHNTIDFLIKACPKQLINVDPYKPRAGYKLWLLFTTISLDQQRELGIEFIPAWKILFTDINMFGSRVPIQFQPAINPLAYVCYLPEDVNVNDGDIVEMRAVDGYDTIPKWELVRSRNDRKNEPGFYGNNYKIASDIYLNYIDVFHFEDLYKYNPGYFEKNKSDIYVAPNKYRRYLIKSLFGRYLRDAKWVIDAAAGRGADLHLYKAECVEHLLAIDIDPTAISELVRRRNEITGYNKSHRGGRNMHSHRGQSHCAKSTSLHALVADLRENPDVLIPKIIQSRPHERCYDAIVINFAIHYLCDTDEHIRDFLITVSRLLAPNGVFIFTTMDGESIVKLLADHKVRPGEAWTIHTGDVNSPDSTVPKYSIRRLYDSDKLTKTGQQIEVLLPMSGEMKAEPLCNIKNIISMARKMGLDLVESANFSVLYEAYARDYPDIYARMTPDDKLYNDLHTYAVFKRKKGAATAGTNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 26 - DNA, 22 bp, synthetic construct
  misc_feature 1..22: note = Synthetic construct
  misc_feature 22: note = 3deSBioTEG
  source 1..22: mol_type = other DNA
cttcttttctctcttatttccc

SEQ ID NO: 27 - DNA, 25 bp, synthetic construct
  misc_feature 1..25: note = Synthetic construct
  modified_base 7: mod_base = OTHER; note = n is uridine
  modified_base 25: mod_base = OTHER; note = n is uridine
  source 1..25: mol_type = other DNA
gggaaanaagagagaaaagaagagn

SEQ ID NO: 28 - DNA, 24 bp, synthetic construct
  misc_feature 1..24: note = Synthetic construct
  modified_base 7: mod_base = OTHER; note = n is uridine
  source 1..24: mol_type = other DNA
gggaaanaagagagaaaagaagag

SEQ ID NO: 29 - DNA, 26 bp, synthetic construct
  misc_feature 1..26: note = Synthetic construct
  modified_base 7: mod_base = OTHER; note = n is uridine
  modified_base 25: mod_base = OTHER; note = n is uridine
  source 1..26: mol_type = other DNA
gggaaanaagagagaaaagaagagna

(19) United States
(12) Patent Application Publication
(10) Pub. No.: US 2023/0287376 A1
(43) Pub. Date: Sep. 14, 2023

(54) IMMOBILIZED ENZYME COMPOSITIONS AND METHODS

(71) Applicant: New England Biolabs, Inc., Ipswich, MA (US)

(72) Inventors: Ming-Qun Xu, Hamilton, MA (US); Guillermo García-Marquina, Peabody, MA (US); Siu-hong Chan, Ipswich, MA (US); Ivan R. Correa, Jr., Hamilton, MA (US); Aihua Zhang, Hamilton, MA (US); Yi Fang, Ipswich, MA (US); Michael Sproviero, Ipswich, MA (US)

(73) Assignee: New England Biolabs, Inc., Ipswich, MA (US)

(21) Appl. No.: 18/182,122

(22) Filed: Mar. 10, 2023

Related U.S. Application Data
(60) Provisional application No. 63/329,262, filed on Apr. 8, 2022.
(60) Provisional application No. 63/319,157, filed on Mar. 11, 2022.

(51) Int. Cl. C12N 9/22; C07K 14/01; C12N 15/09

(52) U.S. Cl. CPC C12N 9/22; C07K 14/01; C12N 15/09

(57) ABSTRACT

The present disclosure relates, according to some embodiments, to immobilized enzyme compositions and methods for cleaving polynucleotide molecules including, for example, double-stranded DNA. Immobilized enzymes may comprise, for example, an enzyme (e.g., a type IIS restriction endonuclease, an RNAP, a capping enzyme), a support (e.g., a magnetic bead), and optionally, a linker disposed between the enzyme and the support. In some embodiments, methods may include contacting an immobilized enzyme with a polynucleotide substrate to form reaction products, separating the immobilized enzyme from the reaction products, and optionally reusing the immobilized enzymes in one or more subsequent reactions. preparing a library for sequencing. For example, a method may comprise (a) in a coupled reaction, (i) contacting a population of nucleic acid fragments with a tailing enzyme to produce tailed fragments, and (ii) ligating to the tailed fragments a sequencing adapter with a ligase to produce adapter-tagged fragments; and/or separating adapter-tagged fragments from the tailing enzyme and the ligase to produce separated adapter-tagged fragments and, optionally, separated tailing enzyme and/or separated ligase.

### CROSS REFERENCE TO RELATED APPLICATION

This application claims the benefit of provisional application Ser. No. 63/319,157, filed on Mar. 11, 2022, and provisional application Ser. No. 63/329,262, filed on Apr. 8, 2022, which applications are incorporated by reference herein in their entirety.

### SEQUENCE LISTING STATEMENT

This disclosure includes a Sequence Listing submitted electronically in .xml format under the file name “NEB-459-US.xml” created on Mar. 10, 2023, and having a size of 67.1 KB This Sequence Listing is incorporated herein in its entirety by this reference.

### BACKGROUND

The manufacture of RNA has received new attention in light of the rise of RNA therapeutics and vaccines, including vaccines against SARS-Cov2. In addition, synthetic RNA may be used to generate pluripotent stem cells or in applications of CRISPR/Cas genome editing technology. In vitro transcription of a linear DNA substrate may be used to produce an RNA having a desired sequence with or without modified nucleotides. RNA produced by IVT may be capped during synthesis via co-transcriptional capping or afterward by post-transcriptional capping. There is a need for compositions, methods, systems and workflows that deliver synthetic RNA efficiently and reliably having the desired yield, length, and sequence.

### SUMMARY

The present disclosure relates, in some embodiments, to an immobilized enzyme comprising a support and an enzyme immobilized thereto. For example, an immobilized restriction enzyme may comprise a type IIS restriction endonuclease, a glycine-serine linker attached to the type IIS restriction endonuclease by a peptide bond, a protein tag (e.g., a SNAP-tag) attached to the linker by a peptide bond, O⁶-benzyleguanine bound to the protein tag (e.g., SNAP-tag®); and magnetic beads having a surface modification comprising the O⁶-benzyleguanine. In some embodiments, a type IIS restriction endonuclease (e.g., BspQI) may have a recognition sequence and cleave site of 5′ GCTCTTC N1/N4 3′. In some embodiments, a type IIS restriction endonuclease (e.g., an engineered type IIS restriction endonuclease, Nt.BspQI) may have a recognition sequence and cleave (nicking) site of 5′ GCTCTTC N1 3′. In some embodiments, a support of an immobilized restriction endonuclease may comprise a magnetic bead. A magnetic bead may comprise, for example, one or more surface modifications. Surface modifications may include, for example, O⁶-benzyleguanine and/or PEG₇₅₀.

The present disclosure relates, in some embodiments, to immobilized BspQI. Immobilized BspQI may comprise, for example, BspQI, a glycine-serine linker attached to the BspQI by a peptide bond, a protein tag (e.g., SNAP-tag®) attached to the linker by a peptide bond, O⁶-benzyleguanine bound to the protein tag (e.g., SNAP-tag®); and magnetic beads having a surface modification comprising the O⁶-benzyleguanine.

The present disclosure relates, in some embodiments, to immobilized RNA polymerase (e.g., T7 RNA polymerase). Immobilized RNA polymerase may comprise, for example, an RNA polymerase (e.g., T7 RNA polymerase), a glycine-serine linker attached to the RNA polymerase by a peptide bond, a protein tag (e.g., SNAP-tag®) attached to the linker by a peptide bond, O⁶-benzyleguanine bound to the protein tag (e.g., SNAP-tag®); and magnetic beads having a surface modification comprising the O⁶-benzyleguanine.

The present disclosure relates, in some embodiments, to immobilized capping enzyme (e.g., FCE). Immobilized RNA polymerase may comprise, for example, a capping enzyme (e.g., FCE), a glycine-serine linker attached to the capping enzyme by a peptide bond, a protein tag (e.g., SNAP-tag®) attached to the linker by a peptide bond, O⁶-benzyleguanine bound to the protein tag (e.g., SNAP-tag®); and magnetic beads having a surface modification comprising the O⁶-benzyleguanine.

The present disclosure relates, in some embodiments, to methods of cleaving a double stranded DNA substrate. Methods may comprise, for example, contacting a first portion of the double stranded DNA substrate with an immobilized enzyme comprising a type IIS restriction endonuclease (e.g., BspQI, Nt.BspQI, BsaI, BsmB, PaqCII) to produce double stranded DNA cleavage products; separating the immobilized enzyme from the double stranded DNA cleavage products to form separated immobilized enzyme and separated double stranded DNA cleavage products; and, optionally, contacting a second portion of the double stranded DNA substrate with the separated immobilized enzyme comprising a type IIS restriction endonuclease to produce more double stranded DNA cleavage products. In some embodiments, methods may comprise repeating the separating and subsequent contacting steps from 2 to 50 times. Methods may comprise, according to some embodiments, combining the separated double stranded DNA cleavage products with the more double stranded DNA cleavage products to produce pooled products.

The present disclosure relates, in some embodiments, to methods of producing an RNA (e.g., a mRNA). Methods may comprise, for example, contacting a first portion of a double stranded DNA substrate with an immobilized enzyme comprising a type IIS restriction endonuclease (e.g., BspQI, Nt.BspQI, BsaI, BsmB, PaqCII) to produce double stranded DNA cleavage products; separating the immobilized enzyme from the double stranded DNA cleavage products to form separated immobilized enzyme and separated double stranded DNA cleavage products; and, optionally, contacting a second portion of the double stranded DNA substrate with the separated immobilized enzyme comprising a type IIS restriction endonuclease to produce more double stranded DNA cleavage products. In some embodiments, methods may comprise repeating the separating and subsequent contacting steps from 2 to 50 times. Methods may comprise, according to some embodiments, combining the separated double stranded DNA cleavage products with the more double stranded DNA cleavage products to produce pooled products. A method may further comprise contacting a first portion of the double stranded DNA cleavage products with a second immobilized enzyme comprising an RNA polymerase (e.g., a type II RNA polymerase, a T7 RNA polymerase) to form an RNA product. A method may further comprise separating the second immobilized enzyme from the RNA product to form separated second immobilized enzyme and separated RNA product; and, optionally, contacting a second portion of the double stranded DNA cleavage products with the separated second immobilized enzyme comprising an RNA polymerase to produce more RNA product. In some embodiments, double stranded DNA cleavage products comprise one or more of a promoter (e.g., a bacteriophage promoter), a 5′ untranslated sequence, a coding sequence, a 3′ untranslated sequence, and a poly(A) sequence (or, in each case, the complement thereof), wherein each sequence is operably linked. In some embodiments, the RNA may comprise, in a 5′-3′ direction, a sequence complementary to the coding sequence and a poly(A) sequence. In some embodiments, a method may further comprise chemically capping the RNA to form a capped RNA product.

Methods of cleaving a double stranded DNA substrate may comprise, in some embodiments, contacting the double stranded DNA substrate with an immobilized enzyme comprising a type IIS restriction endonuclease (e.g., BspQI) to produce double stranded DNA cleavage products, wherein the double stranded DNA cleavage products comprise at least one nick; separating the immobilized enzyme from the double stranded DNA cleavage products to form separated immobilized enzyme and separated double stranded DNA cleavage products; and contacting the separated double stranded DNA cleavage products with a second enzyme. A double stranded DNA substrate may comprise on the non-template strand, in a 5′-3′ direction, a phage polymerase promoter sequence (e.g., a T7 promoter, an SP6 promoter), a 5′ untranslated region a coding sequence, a 3′ untranslated region an optional poly(A) sequence, and a type IIS restriction recognition site (e.g., a BspQI recognition site). The template strand of the template comprises of the complementary sequence of the non-template strand.

The present disclosure relates to methods of producing RNA, according to some embodiments. Methods of producing RNA may comprise, for example, (a) contacting a polynucleotide template with an immobilized enzyme to form a cleaved polynucleotide template; and (b) contacting the cleaved polynucleotide template with an RNA polymerase (e.g., a type II RNA polymerase, a phage RNA polymerase) to produce transcription products comprising the RNA. A polynucleotide template may comprise on the non-template strand, in a 5′-3′ direction, a phage polymerase promoter sequence (such as a T7 promoter or a SP6 promoter), a 5′ untranslated region, a coding sequence, 3′ untranslated region, an optional poly(A) sequence, and a type IIS restriction recognition site (e.g., a BspQI recognition site). The template strand of the template comprises of the complementary sequence of the non-template strand. An immobilized enzyme may comprise a type IIS restriction endonuclease (e.g., BspQI), a support, and a linker disposed between the type IIS restriction endonuclease and the support. For example, an immobilized enzyme may comprise immobilized BspQI. In some embodiments, the (a) contacting and the (b) contacting steps may be performed as a coupled reaction. According to some embodiments, the RNA produced comprises, in a 5′-3′ direction, a sequence based on the template sequence of an optional poly(A) sequence. In some embodiments, methods may further comprise contacting the RNA with a capping enzyme to form a capped RNA product (e.g., co-transcriptionally or post-transcriptionally). Methods may further comprise, in some embodiments, chemically capping the RNA to form a capped RNA product.

The present disclosure provides, according to some embodiments, methods of performing in vitro transcription. For example, a method may comprise (a) contacting a double stranded DNA template with a first enzyme to form a nicked template, the first enzyme comprising a type IIS restriction endonuclease (e.g., BspQI, Nt.BspQI); (b) contacting the nicked template with a second enzyme to form a transcription product, the second enzyme comprising an RNA polymerase (e.g., type IIS restriction endonuclease, T7 RNA polymerase); and (c) optionally contacting the transcription product with a third enzyme to form a capped transcription product, the third enzyme comprising a capping enzyme (e.g., Faustovirus capping enzyme), wherein at least one of the first enzyme, the second enzyme, and the third enzyme are immobilized enzymes. In some embodiments, contacting the transcription product with the capping enzyme further comprises contacting the transcription product with a Cap-0 capping enzyme (e.g., Faustovirus capping enzyme) to form a Cap-0 transcription product and optionally contacting the Cap-0 transcription product with a Cap-1 capping enzyme (e.g., 2′OMTase) to form a Cap-1 transcription product. Capping efficiency for an in vitro transcription reaction may be at least 40%, at least 50%, at least 60%, at least 70%, at least 80%, at least 90%, at least 95%, at least 97%, or up to 100%.

The present disclosure relates, in some embodiments, to an IVT reactor system. An IVT reactor system may comprise a first reactor having an inlet, an outlet, and a reaction chamber disposed between the inlet and the outlet. A first reactor may comprise one or more enzymes, each independently selected from a soluble enzyme, a soluble fusion protein comprising one or more enzymes, an immobilized enzyme, and an immobilized fusion protein comprising one or more enzymes. A first reactor may comprise, for example,
a soluble type IIS restriction endonuclease (e.g., BspQI),
a soluble RNA polymerase (e.g., T7 RNA polymerase, SP6 RNA polymerase, T3 RNA polymerase or variants thereof),
a soluble capping enzyme (e.g., VCE, FCE, 2′OMT),
a soluble fusion protein comprising an RNA polymerase (e.g., T7 RNA polymerase) and a capping enzyme (e.g., FCE),
an immobilized type IIS restriction endonuclease (e.g., BspQI),
an immobilized RNA polymerase (e.g., T7 RNA polymerase, SP6 RNA polymerase, T3 RNA polymerase or variants thereof),
an immobilized capping enzyme (e.g., VCE, FCE, 2′OMT)),
an immobilized fusion protein comprising an RNA polymerase and a capping enzyme, or
combinations thereof,
   optionally wherein the immobilized type IIS restriction endonuclease, the immobilized RNA polymerase, or the immobilized capping enzyme is co-immobilized on a support with one or both of the other two enzymes. For example, a first reactor may comprise (a) a soluble RNA polymerase or an immobilized RNA polymerase or (b) a soluble capping enzyme or an immobilized capping enzyme or (c)(i) a soluble RNA polymerase or an immobilized RNA polymerase and (ii) a soluble capping enzyme or an immobilized capping enzyme. Each included enzyme may be a soluble enzyme, a soluble fusion protein, an immobilized enzyme, or an immobilized fusion protein, wherein immobilized enzymes may be immobilized, for example, on a magnetic bead or agarose support). According to some embodiments, an IVT reactor system may comprise a first reactor and a second reactor, for example, wherein the first reactor comprises at least one enzyme and the second reactor comprises at least one enzyme, wherein the first reactor enzyme(s) differ from the second reactor enzyme(s). For example, a first reactor may comprise a soluble type HS restriction endonuclease (e.g., BspQI) or an immobilized type IIS restriction endonuclease (e.g., BspQI) and a second reactor may comprise (a) a soluble RNA polymerase or an immobilized RNA polymerase or (b) a soluble capping enzyme or an immobilized capping enzyme or (c)(i) a soluble RNA polymerase or an immobilized RNA polymerase and (ii) a soluble capping enzyme or an immobilized capping enzyme. A first reactor may comprise (a) a soluble RNA polymerase or an immobilized RNA polymerase or (b) a soluble Cap-0 capping enzyme or an immobilized Cap-0 capping enzyme or (c)(i) a soluble RNA polymerase or an immobilized RNA polymerase and (ii) a soluble Cap-0 capping enzyme or an immobilized Cap-0 capping enzyme and a second reactor may comprise a soluble Cap-1 capping enzyme or an immobilized Cap-1 capping enzyme. An IVT reactor system may further comprise one or more pumps operably linked to one or more reactors, one or more feed chambers (e.g., comprising substrate dsDNA, NTPs, buffering agents, one or more soluble enzymes) in fluid communication with a reactor (e.g., via the reactor inlet). A reactor system may include a reactor and one or more heating elements and/or cooling elements in thermal communication with the reactor. An apparatus or system may comprise, for example, a fraction collector in fluid communication with the outlet of a reactor. The present disclosure also relates to compositions comprising the foregoing components of reactor systems without, for example, the reactors, inlets, outlets, chambers, pumps, fraction collector and the like.

### BRIEF DESCRIPTION OF THE FIGURES

FIGS. 1A-1E schematically illustrate five methods for preparing nucleic acid libraries for sequencing (e.g., Nanopore MinION® sequencing). FIG. 1A shows library construction in two sequential enzymatic steps using soluble enzymes without AMPure® bead purification (Sol without BP). FIG. 1B shows library construction in two sequential enzymatic steps using soluble enzymes with AMPure® bead purification (Sol-seq). FIG. 1C shows library construction in a coupled enzymatic reaction using soluble enzymes with AMPure® bead purification (Sol-cpl). FIG. 1D shows library construction in two sequential enzymatic steps using immobilized enzymes without bead purification (Im-seq). FIG. 1E shows library construction in a coupled enzymatic reaction using immobilized enzymes without bead purification (Im-cpl).

FIG. 2 shows that poly(A) extension is dependent on poly(A) polymerase concentration. An RNA 45-mer oligo strand was treated with different concentrations of untagged poly(A) polymerase from NEB and poly(A) tailing activity of poly(A) polymerase was evaluated by capillary electrophoresis (CE).

FIG. 3 shows that poly(A) extension is dependent on the concentration of poly(A) polymerase—SNAP fusion protein. An RNA 45-mer oligo strand was treated with different concentrations of a poly(A) polymerase—SNAP fusion protein and poly(A) tailing activity of was evaluated by capillary electrophoresis (CE).

FIG. 4 shows poly(A) polymerase immobilization on PEG₇₅₀ coated O⁶-benzylguainine (BG) beads.

FIG. 5 shows that poly(A) extension is dependent on the concentration of an immobilized poly(A) polymerase. An RNA 45-mer oligo strand was treated with different concentrations of an immobilized poly(A) polymerase and poly(A) tailing activity was evaluated by capillary electrophoresis (CE).

FIG. 6 shows that poly(A) tailing activity from soluble or immobilized SNAP-tagged poly(A) polymerase (PAP). The following four reaction samples (from top to bottom) were analyzed by CE technique: Sample 1, 45-mer RNA oligo substrate without enzymatic treatment; Sample 2, substrate treated with soluble PAP; Sample 3, substrate reacted with PAP immobilized to BG-magnetic beads with no PEG₇₅₀-coating; sample 4: substrate reacted with PAP immobilized to PEG₇₅₀-coated BG magnetic beads. Sample 2 and Sample 4 displayed poly(A) tailing activity.

FIG. 7 shows relative activity of T4 DNA ligase immobilized on magnetic beads stored at −20° C. or 25° C. over a period of 7 days. The activity at day 1 is normalized to 100%.

FIG. 8 shows that T4 DNA ligase immobilized on agarose beads is more thermostable than two soluble T4 DNA ligases. Heat treatment was conducted with two soluble T4 DNA Ligases, untagged T4 DNA Ligase (NEB M0202) and SNAP-tagged T4 DNA Ligase (HS-T4 DNA Ligase), and immobilized T4 DNA Ligase (HS-T4 DNA Ligase-Agarose). Ligase activity was monitored using FMA-labeled synthetic double-stranded DNA (FAM-dsDNA). The substrate/enzyme mixtures were treated at various temperatures (40° C.-100° C.), followed by incubation at 4° C. overnight. Fluorescent gel scanning was used to visualize substrate and ligation products (including the major product, termed Product), as detected in positive controls when the reactions were pre-treated at 4° C. but were absent in the negative controls (NO Enzyme).

FIGS. 9A-9C show that SNAP-tagged T4 DNA Ligase immobilized onto agarose beads displayed ligase activity after heat treatment at 45° C. for 30 min but the soluble form showed little or no ligase activity under the same conditions. Aliquots of HS-T4 DNA Ligase conjugated to BG-Agarose beads were incubated for 30 min at 4° C. (A), 37° C. (B) or 45° C. (C), followed by ligation reactions at room temperature (23° C.) for 2 hours. The samples (in the same order) were loaded onto three PAGE gels for electrophoretic separation, followed by fluorescent gel scanning. 1, No Enzyme; 2, Ligase (untagged T4 DNA Ligase, NEB M0202); 3, HS-Ligase (SNAP-tagged T4 DNA Ligase); 4, Ligase-Agarose; 5, Ligase-Chitin; 6, Ligase-Mag; 7, Ligase-SiM. Arrows indicate the expected positions of ligation product for soluble T4 DNA Ligase (arrows in lane 2) or Ligase-Agarose (arrows in lane 4).

FIGS. 10A-10B show that SNAP-tagged T4 DNA Ligase immobilized onto agarose beads displayed more ligase activity after heat treatment at 55° C. or 65° C. than the soluble form, which had little or no ligase activity under the same conditions. DNA ligation was monitored using a fluorophore (FAM)-labeled DNA substrate. The reactions were incubated for 10 min at 4° C., 55° C. or 65° C., followed by ligation reactions at 23° C. for 2 hours (FIG. 10A) or at 4° C. overnight (FIG. 10B). The samples were electrophoresed on PAGE gels, followed by fluorescent gel scanning. Ligase, untagged T4 DNA Ligase, NEB M0202; HS-Ligase, SNAP-tagged T4 DNA Ligase; Ligase-Agarose, HS-T4 DNA Ligase immobilized to BG-Agarose Beads; Ligase-Mag, HS-T4 DNA Ligase conjugated to BG-Magnetic Beads. Arrows indicate the major ligation product from the reactions with Ligase-Agarose.

FIG. 11 shows that immobilized enzymes can be reused for consecutive reactions. Data from reactions #1, #10, and #20 of twenty consecutive ligation reactions catalyzed by a single preparation of immobilized T4 DNA ligase. Performance over these twenty reactions matches the results observed in a single reaction with soluble T4 DNA ligase.

FIG. 12 shows number of direct RNA sequencing reads from libraries prepared by two methods. In the first, labeled Sol-seq, the library was prepared by two sequential steps of poly(A) tailing and adaptor ligation. In the second, labeled Sol-cpl, the library was prepared by carrying out poly(A) tailing and adaptor ligation simultaneously.

FIG. 13 shows the poly(A) tailing and adaptor ligation activity from immobilized poly(A) polymerase and immobilized T4 DNA Ligase. Four samples were examined by CE (from top to bottom): Sample 1, untreated FAM-labeled RNA substrate showing a distinct peak; sample 2, FAM-labeled RNA substrate treated by immobilized Poly(A) polymerase; Sample 3, Sample 2 treated with immobilized T4 DNA ligase and RTA-poly(dT)₁₅; Sample 4, Sample 2 treated with immobilized T4 DNA ligase and RTA-poly(dT)₁₀. A bell-shaped peak in Sample 2 represents addition of 3′ poly(A) tails of various length to the RNA substrate (Sample 1). Ligation of an RTA adaptor to the poly(A) tailed products generated higher molecular mass products resulting in a shift of the bell-shaped peak to the right.

FIG. 14 compares Nanopore RNA sequence reads obtained with libraries prepared by different methods. Each library was prepared using soluble enzymes without bead purification (Sol w/o BP), soluble enzymes with sequential poly(A) tailing and ligation with bead purification (Sol-seq), or immobilized enzymes with sequential poly(A) tailing and ligation protocol without bead purification (Im-seq). 164 ng of RNA library was used for each sequencing run.

FIG. 15 shows total sequence reads from Nanopore direct RNA sequencing of RNA libraries constructed with 500 ng input RNA using immobilized enzymes without AMPure® bead purification following either sequential reaction protocol (Im-seq) or coupled reaction protocol (Im-cpl). After enzymatic treatment enzyme-conjugated beads were pelleted on a magnetic rack and the supernatants were transferred to a fresh tube. 105 ng of total RNA from each library was mixed with the solution provided by ONT before loading onto MinION® R9.1.4 flow cells for direct RNA sequencing.

FIG. 16A-16B shows that co-immobilized enzymes displayed both poly(A) polymerase activity and T4 DNA ligase activity. FIG. 16A shows that immobilized poly(A) polymerase, co-immobilized with T4 DNA ligase, is active in a poly(A) tailing assay in which a poly(A) tail is added to a 35-mer RNA (lower panel), but not a corresponding control (upper panel). FIG. 16B shows that immobilized T4 DNA ligase, co-immobilized with poly(A) polymerase on BG-modified beads (BGPL), displayed activity in an adapter ligation assay in which adapters RTA and RMX were ligated to each other (lower panel), but not a control with RTA alone (upper panel).

FIG. 17 shows an example of fully automated Nanopore sequencing workflow which includes library construction catalyzed by immobilized enzymes.

FIG. 18A-18B shows the number of Nanopore RNA sequencing reads obtained with low-input RNA libraries prepared with immobilized enzymes. FIG. 18A shows the number of sequencing reads obtained from 100 ng of Listeria monocytogenes RNA libraries prepared using immobilized enzymes following either a sequential reaction method (“Im-seq 100”; Example 4D) or a coupled reaction method (“Im-cpl 100”; Example 4E) without AMPure® bead purification. FIG. 18B shows the number of sequencing reads obtained from 100 ng of mammalian RNA libraries prepared using immobilized ligase (“ImL”) or immobilized polymerase and immobilized ligase (“ImP & ImL”) without AMPure® bead purification. Each output library was loaded onto Nanopore R9.4.1. flow cells for direct RNA sequencing.

FIG. 19 shows a comparison of the number of Nanopore RNA sequencing reads obtained with RNA libraries prepared according to one of five methods: Sol-seq libraries were prepared with soluble enzymes and bead purification using a sequential reaction protocol; Sol-cpl libraries were prepared with soluble enzymes and bead purification using a coupled reaction protocol; Sol w/o BP libraries prepared with soluble enzymes sequentially without bead purification; Im-seq libraries were prepared with immobilized enzymes using the sequential reaction protocol without bead purification; Im-cpl libraries were prepared with immobilized enzymes using the coupled reaction protocol without bead purification. The sequencing reads shown were obtained after 2 hour run time.

FIG. 20 shows a comparison of functional nanopores over time during direct RNA sequencing runs in ONT flow cells. Duplicate libraries were prepared using each of the five protocols illustrated in FIGS. 1A-1E and the resulting sequencing reads were displayed in FIG. 19.

FIG. 21 shows the sequence reads per nanopore from libraries prepared by the coupled reaction method (square dots between 900 and 1100 of normalized reads) in comparison with those from the libraries prepared by the sequential reaction method with immobilized enzymes (circular dots between 200 and 500 of normalized reads). The upper curve aligns the reads/pore data points from three sequencing samples with 83 ng, 109 ng or 136.5 ng loaded on a flow cell. The lower curve aligns the reads/pore data points from four sequencing samples with various amount of RNA (38 ng, 39 ng, 105 ng and 164.4 ng) per flow cell.

FIG. 22 shows proposed DNA library preparation workflow for ONT sequencing platform. The workflow is comprised of three reactions, poly(dA) tailing catalyzed by terminal deoxynucleotidyl transferase (TdT), ligation of a 3′ Poly(dT)-containing adaptor with motor protein (dot), and gap-filling and nick sealing with DNA polymerase (Pol) and DNA Ligase. Relevant soluble or immobilized enzymes can be utilized to catalyze each enzymatic treatment. Enzymes may be removed, inactivated or present in the final sequencing library.

FIG. 23 shows a two-step reaction of poly(dA) tailing of a synthetic double-stranded DNA substrate (possessing a FAM probe) catalyzed by TdT and subsequent ligation with a synthetic adaptor, RTA-poly(dT) possessing a ROX probe. A ligated product can be detected by CE analysis due to the presence of both FAM and ROX probes (as shown in FIG. 18C).

FIG. 24A-24C shows sequential poly(dA) and ligation reactions with immobilized enzymes. FIG. 24A shows poly(dA) tailing of 5′FAM-labeled DNA substrate by TdT in two different substrate-to-dATP ratios (1:100 and 1:200). Incorporation of dAMP at the 3′ termini of 5′FAM-labeled DNA strand and the length or range of poly(dA) can be detected by CE analysis. FIG. 24B shows detection of a FAM-labeled DNA substrate ligated to ROX-labeled RTA-Poly(dT) using FAM-detecting channel by CE analysis. Top: FAM-labeled DNA substrate; Middle: FAM-labeled DNA substrate treated with TdT shows multiple species corresponding to various poly(dA) length; Bottom: the Poly(dA)-tailed DNA mixture further treated with T4 DNA Ligase in the presence of RTA-Poly(dT) exhibits a shift to a pool of higher molecular mass species with various length of poly(dA) tails. FIG. 24C shows detection of a FAM-labeled DNA substrate ligated to ROX-labeled RTA-Poly(dT) by CE analysis. Top: FAM-labeled DNA substrate without enzyme treatment; Middle: FAM-labeled DNA substrate treated with TdT shows multiple species corresponding to various length of poly(dA) tails; Bottom: detection of the ligation products using both FAM- and ROX-detecting channels (depicted in dark and light, respectively). The Poly(dA)-tailed DNA mixture (as presented in the middle graph) treated with T4 DNA Ligase in the presence of RTA-Poly(dT) exhibits a shift to a pool of higher molecular mass species with various length of poly(dA) tails, with overlapping signals from FAM and ROX probes.

FIG. 25 shows CE analysis of sequential Poly(dA) tailing and adaptor ligation reaction products catalyzed by soluble and immobilized T4 DNA ligase. FAM-labeled DNA substrate ligated to ROX-labeled RTA-Poly(dT) by TdT in a substrate-to-dATP ratio of 1:100. Subsequently, the reaction medium containing the poly(dA)-tailed DNA products (pool), was incubated with either soluble or immobilized T4 DNA ligase and RTA-poly(dT) adaptor possessing 3′ poly(dT) and ROX probe. DNA substrate: FAM-labeled DNA substrate without enzyme treatment; TdT: FAM-labeled DNA substrate treated with TdT showing multiple species corresponding to various length of poly(dA) tails; TdT+Ligase: Poly(dA) tailed DNA treated by T4 DNA Ligase was examined with both FAM- and ROX-detecting channels (depicted in dark and light gray, respectively). TdT+IM-Ligase: TdT-treated DNA was treated with immobilized T4 DNA Ligase and examined with both FAM- and ROX-detecting channels (depicted in dark and light gray, respectively). RTA-Poly(dT): adaptor without enzymatic treatment. Co-localization of the fluorescence signals of FAM (dark gray) and ROX (light gray) indicates ligation of the 5′ FAM-labeled DNA pool to the 3′ ROX-labeled strand of the adaptor.

FIG. 26 shows schematic diagrams of two methods of DNA library construction. FIG. 26A shows library construction using soluble enzymes with an AMPure® bead purification step. FIG. 26B shows library construction using immobilized DNA modifying enzymes without AMPure® bead purification.

FIG. 27 shows end repair, dA-tailing and adaptor ligation of synthetic DNA modified using immobilized enzymes with products of each step subjected to CE analysis. This method is designed for construction of Nanopore DNA library without use of AMPure® bead purification and PEG-based buffer.

FIG. 28 shows a diagram of a sequence (SEQ ID NO:12) comprising an example BspQI recognition (5′GCTCTTC3′, underlined) and cleavage sites (arrows). BspQI cleavage of a typical sequence shown can generate a DNA template for in vitro synthesis of RNA having a 3′poly(A) tail.

FIGS. 29A-29B show schematic diagrams of example SNAP-tagged BspQI fusion proteins. FIG. 29A shows a diagram of a 6×His-SNAP-BspQI fusion. FIG. 29B shows a 6×His-SNAP-(GS)₆-BspQI fusion. Here, (GS)₆ represents a repetitive Gly-Ser peptide linker. Upon incorporation into an immobilized enzyme, this linker would be positioned between SNAP-tag and the support.

FIGS. 30A-30F show embodiments of an immobilized enzyme, namely SNAP-tagged BspQI fusion proteins immobilized onto magnetic beads. Two types of recombinant fusion proteins, 6×His-SNAP-tag-BspQI (FIG. 30A, FIG. 30B and FIG. 30C) and 6×His-SNAP-tag-GSx6-BspQI (FIG. 30D, FIG. 30E and FIG. 30F) were used for covalent conjugation onto three types of 1 um of magnetic beads (possessing benzylguanine ligand), respectively—BG modified magnetic beads (FIG. 30A and FIG. 30D), BG modified magnetic beads with PEG₇₅₀ coating (FIG. 30B and FIG. 30E), and BG modified magnetic beads with PEG₇₅₀ coating and PEG4 spacer (FIG. 30C and FIG. 30F).

FIG. 31 shows results of example enzyme activity assays for immobilized BspQI.

A 1 Kb DNA ladder (N3270S, New England Biolabs, Inc.) was used for size standards (Lane 1 and 16). λ phage DNA (N3011L, New England Biolabs, Inc.) substrate without enzymatic treatment was used as negative control (Lane 2) and λ phage DNA digested with NEB BspQI was used as positive control (Lane 15). λ phage DNA was treated under the same conditions with a variety of immobilized BspQI. 6×His-SNAP-tag-BspQI (“HS-BspQI”, Lanes 3 to 8) and 6×His-SNAP-tag-GSx6-BspQI (“HS-GSx6-BspQI”, Lanes 9 to 14) were immobilized onto BG modified magnetic beads (Lanes 3, 4, 9 and 10), BG modified magnetic beads with PEG₇₅₀ coating (Lanes 5, 6, 11 and 12) and BG modified magnetic beads with PEG₇₅₀ coating and PEG₄ spacer (Lanes 7, 8, 13 and 14). For each enzyme-bead combination, the assays were repeated for 10-fold dilution (Lanes 3, 5, 7, 9, 11 and 13) and 100-fold dilution (Lanes 4, 6, 8, 10, 12 and 14) of immobilized enzymes.

FIG. 32 shows an example comparison of enzymatic activities of an immobilized HS-GSx6-BspQI and a corresponding soluble BspQI. A 1 Kb DNA ladder was used as standards (Lane 1 and 20). Untreated λ DNA was used as negative control (Lane 2 and 19). Serial 2-fold dilutions were performed for both soluble BspQI (NEB N0712L, “NEB BspQI”, Lane 3-10) and immobilized BspQI (6×His-SNAP-tag-GSx6-BspQI, “BG-GSx6-BspQI”, Lane 11-18).

FIGS. 33A-33C show example workflows for in vitro transcription mediated by immobilized enzymes. Workflows may include one or more reactors. Reactors may have any desired shape but are shown here as cylinders packed with immobilized enzymes on beads. Insets associated with reactors show enzymes as irregular curvilinear shapes on circular (representing generally spherical) beads. FIG. 33A shows a continuous workflow for in vitro RNA synthesis using one or more immobilized enzymes. Plasmid dsDNA substrate is shown entering a first reactor comprising immobilized BspQI (an example of a type IIS endonuclease) to produce linear dsDNA, which then enters a second reactor comprising immobilized T7 RNA polymerase (“RNAP”; an example RNA polymerase) to produce mRNA, which then enters a third reactor comprising immobilized FCE (an example capping enzyme). The enzymatic treatments may be performed consecutively (as shown) or concurrently. For example, T7 RNAP and FCE may be utilized in a one-pot formulation (e.g., by packing T7 RNAP beads and FCE beads in a single reactor). FIG. 33B shows such a workflow using enzymes co-immobilized onto the same microbead, for example, T7RNAP and capping enzyme FCE. FIG. 33C shows a workflow in which T7 RNAP and FCE are expressed as a fusion protein and immobilized together.

FIGS. 34A-34F show example workflows for in vitro transcription mediated by immobilized enzymes. Enzymes are shown as clusters of dots on circular beads. Enzymes may be included in one or more reactors consistent with FIGS. 33A-33C. FIG. 34A shows a cap structure in which the methyl groups that define Cap-0 and Cap-1 RNA structures are circled. Cap-1 has methyl group in the 2′-O position. FIG. 34B shows an example continuous workflow for in vitro RNA synthesis using consecutive reactions involving immobilized enzymes: BspQI generates linear DNA template (e.g., from a closed circular dsDNA substrate), T7 RNAP catalyzes in vitro transcription to produce non-capped RNA, FCE converts the non-capped RNA to Cap-0 product, and 2′OMTase adds a methyl group to the Cap-0 product to form Cap-1 product. FIG. 34C shows an example workflow comprising contacting a linear dsDNA template with T7RNAP and FCE co-immobilized onto the same microbead to achieve one-pot co-transcriptional capping. The workflow shown optionally includes contacting a circular dsDNA with immobilized BspQI to produce the linear dsDNA template and/or contacting the Cap-0 product with an immobilized 2′-O-methyltransferase to form Cap-1 product. FIG. 34D shows an example workflow comprising contacting a T7RNAP, FCE and 2′OMTase co-immobilized onto the same microbead to achieve one-pot co-transcriptional capping and second methylation. The workflow shown optionally includes contacting a circular dsDNA with immobilized BspQI to produce the linear dsDNA template. FIG. 34E shows an example workflow comprising contacting a linear dsDNA template with immobilized fusion protein SNAP-FCE-T7 RNAP to achieve one-pot co-transcriptional capping. The workflow shown optionally includes contacting a circular dsDNA with immobilized BspQI to produce the linear dsDNA template and/or contacting the Cap-0 product with an immobilized 2′-O-methyltransferase to form Cap-1 product. FIG. 34F shows an example workflow comprising contacting a linear dsDNA template with a SNAP-FCE-T7 RNAP fusion protein and 2′OMTase co-immobilized onto the same microbead to achieve one-pot co-transcriptional capping and second methylation. The workflow shown optionally includes contacting a circular dsDNA with immobilized BspQI to produce the linear dsDNA template.

FIGS. 35A-35B show an example comparison of enzymatic activity of soluble and immobilized BspQI and monitoring of BspQI immobilization by examination of enzymatic activity before and after immobilization and wash. FIG. 35A shows a schematic of an example reaction in which λ DNA (comprising 10 BspQI restriction sites) is contacted with immobilized BspQI (“BspQI@BG”) to produce λ DNA fragments. FIG. 35B shows results of cutting λ DNA with BspQI in which the lanes contain the following: lanes 1, 20, 21 and 40: 1 kb molecular weight ladder (NEB #B7025); lane 2: λDNA negative control; lanes 3-9: series of dilution of SNAP-BspQI with dilution factor of 2; lanes 10-16: series of dilutions of NEB BspQI with dilution factor of 2; lanes 22-28: series of dilution of SNAP-BspQI loaded with dilution factor of 2; lanes 29-35: series of dilution of immobilized SNAP-BspQI with dilution factor of 2; lanes 17-19 & 36-39: series of dilution of SNAP-BspQI magnetic beads flow-through with dilution factor of 2.

FIGS. 36A-36D show enzymatic activity of example soluble and immobilized BspQI. Titrations and reactions were carried out for soluble SNAP-tagged BspQI (SNAP-BspQI) in 3.1 buffer (FIG. 36A) and in T7 RNAP buffer (FIG. 36B), immobilized SNAP-tagged BspQI (BspQI@BG) (FIG. 36C) and soluble untagged BspQI (NEB BspQI) (FIG. 36C). The relative activity level at various reactions were shown as a percentage of substrate and product. The control reactions (Control) contained no enzyme addition.

FIG. 37 shows an agarose gel electrophoresis analysis of digestion of λDNA by an example immobilized BspQI in a continuous flow reactor in which the lanes contain the following: lane 1: negative control, lanes 2-4: NEB BspQI positive control (10,000 U/mL in 1:1; 1:2 and 1:4 serial dilutions), lanes 5-14: continuous process fractions (consecutive collections by changing the tube every 5 min), and lane 15: 1 kb molecular ladder (NEB #B7025).

FIG. 38 shows an agarose gel electrophoresis analysis of digestion of λDNA by an example immobilized BspQI in a continuous flow reactor in which the lanes contain the following: lane 1: negative control, lanes 2-4: NEB BspQI positive control (10,000 U/mL in 1:1; 1:2 and 1:4 serial dilutions), lanes 5-11: continuous process fractions (consecutive collections by changing the tube every 96 min), lane 12: 1 kb molecular ladder (NEB #B7025).

FIG. 39 shows an agarose gel electrophoresis analysis of digestion of λDNA by an example immobilized BspQI in a continuous flow reactor in which the lanes contain the following: lane 1: negative control, lanes 2-4: NEB BspQI positive control (10,000 U/mL in 1:1; 1:2 and 1:4 serial dilutions), lanes 5-18: continuous process fractions (consecutive collections by changing the tube every 71 min)

FIG. 40 shows an analysis of immobilized BspQI reactor in continuous flow. The sketch illustrates a process in which a volume of λDNA substrate reacted with immobilized BspQI in a column reactor over a 24 hour period concluding with collection of a first fraction (“1”) followed by additional volumes over subsequent 24 hour periods to form additional fractions (“2”-“6”). The lanes contain the following: lane 1 corresponds to the last fraction collected after the initial 24 hour reaction (i.e., the last fraction collected in experiment 3 of Example 22), lanes 2-6 correspond to 3.5 mL fractions collected every 24 h (collection time for each fraction is 60 min), and lane 7 is a 1 kb molecular ladder (NEB #B7025).

FIG. 41 shows an analysis of immobilized enzyme leaching in which the lanes contain the following: lane 1: negative control, lanes 2-4: NEB BspQI positive control (10,000 U/mL in 1:1; 1:2 and 1:4 serial dilutions), lanes 5-18: pUC19 digestion reactions performed with continuous process fractions collected in Experiment 22C, and lane 19: 1 kb molecular ladder (NEB #B7025).

FIG. 42 shows an agarose gel electrophoresis analysis of digestion of pUC19 DNA by an example immobilized BspQI in a continuous flow reactor in which the lanes contain the following: lane 1-6: NEB BspQI positive control (10,000 U/mL in 1:1; 1:2; 1:4; 1:8; 1:16 and 1:32 serial dilutions), lanes 5-18, lane 7: negative control, lanes: 8-17: continuous flow process fractions (consecutive collections by changing the tube every 81 min). Lane 18: 1 kb molecular ladder (NEB #B7025). Box indicated linearized pUC19 DNA.

FIG. 43 shows a comparison of enzymatic activity of BspQI immobilized on magnetic or agarose beads in which the lanes contain the following: lane 1: positive control, pUC19 DNA digested with soluble BspQI (NEB BspQI at 10,000 U/mL). lane 2: negative control, pUC19 DNA without BspQI treatment. lanes 3-9: reactions with BspQI@MG-BG serial dilutions (1:1; 1:2; 1:4; 1:8; 1:16; 1:32 and 1:64). lane 10: 1 kb ladder (NEB #B7025). lanes 11-17: reactions with BspQI@AG-BG serial dilutions (1:1; 1:2; 1:4; 1:8; 1:16; 1:32 and 1:64). The hollow arrow indicates linearized pUC19 product whereas the solid arrow exhibits the undigested pUC19 DNA.

FIG. 44 shows an agarose gel electrophoresis analysis of digestion of pUC19 DNA by an example immobilized BspQI in a continuous flow reactor in which the lanes contain the following: lanes 1-10: continuous flow process fractions (consecutive collections by changing the tube every 81 min), lane 11: negative control, pUC19 DNA in the absence of BspQI treatment, lane 12: 1 kb molecular ladder (NEB #B7025). Box indicates linearized pUC19 DNA.

FIGS. 45A and 45B show comparative thermostability of soluble and immobilized BspQI. BspQI samples were incubated at various temperatures indicated and subsequently used for digestion of FAM-labeled DNA substrate followed by CE analysis. FIG. 45A shows relative efficiency of substrate conversion by three BspQI enzyme samples, soluble SNAP-BspQI (Free BspQI), SNAP-tagged BspQI immobilized to BG magnetic beads (BspQI@MG-BG) and SNAP-BspQI immobilized to BG agarose beads (BspQI@AG-BG). FIG. 45B displays the decrease of BspQI enzymatic activity over time at 60° C. for the three samples examined.

FIGS. 46A and 46B show production of DNA template (for IVT) by soluble and immobilized BspQI coupled with in vitro transcription. FIG. 46A shows agarose gel electrophoresis of pRNA21 treated by either soluble or immobilized BspQI in which the lanes contain the following: lanes 1-3: positive control, BspQI digested pRNA21 (NEB BspQI, 10,000 U/mL in 1:1, 1:2 and 1:4 dilutions), lanes 4-6: pRNA21 digestion with BspQI@MG-BG serial dilutions (1:1; 1:2 and 1:4), lane 7: negative control, pRNA21 without enzyme treatment, and lane 8: 1 kb molecular ladder. FIG. 46B shows yield of in vitro transcription by T7 RNA polymerase following template production. The pRNA21 sample digested by soluble BspQI was subjected to conventional DNA purification. For the pRNA21 sample treated by immobilized BspQI, the BspQI@MG-BG beads were pelleted on a magnetic rack and the supernatant was directly mixed used for IVT by T7 RNA polymerase without a conventional purification step.

FIGS. 47A and 47B show in vitro transcription with immobilized T7 RNAP at different protein loads both with sera magnetic and agarose beads. FIG. 47A shows agarose gel electrophoresis of in vitro transcription products in which the lanes contain the following: lanes 1-5: IVT reactions for 0.2 mg/mL enzyme load, with load, T7@MG-BG, flow-through of T7@MG-BG, T7@AG-BG and flow-through of T7@AG-BG; lanes 6-10: IVT reactions for 0.5 mg/mL enzyme load, with load, T7@MG-BG, flow-through of T7@MG-BG, T7@AG-BG and flow-through of T7@AG-BG; lanes 11-16: IVT reactions for 1 mg/mL enzyme load, with load, T7@MG-BG, flow-through of T7@MG-BG, T7@AG-BG and flow-through of T7@AG-BG; and lane 17: ssRNA ladder.

FIGS. 48A-48C show in vitro mRNA capping efficiency of soluble and immobilized FCE by capillary electrophoresis. mRNA capping efficiency of serial 1:2 dilutions of soluble FCE (FIG. 48A), FCE immobilized in benzylguanine-coated sera magnetic beads (FIG. 48B) and FCE immobilized in benzylguanine and PEG750-coated sera magnetic beads (FIG. 48C). m7Gppp- is the final product (N7 methylase product), Gppp- is the product of the second enzyme activity (guanylyl transferase), p- is a byproduct of chemical synthesis of the RNA that is not reactive in capping reaction, pp- is the product of the first enzyme activity (triphosphatase) and ppp- is the substrate.

FIG. 49 shows SDS-PAGE of SNAP-T7 RNAP, SNAP-FCE and SNAP-2′OMTase co-immobilization in BG-PEG750-coated sera magnetic beads in which the lanes contain the following: lane 1: protein standard (NEB, P7719S); lane 2: SNAP-T7 RNAP+SNAP-FCE load; lane 3: SNAP-T7 RNAP+SNAP-FCE flow-through; lane 4: SNAP-T7 RNAP+SNAP-FCE+SNAP-2′OMTase load; and lane 5: SNAP-T7 RNAP+SNAP-FCE+SNAP-2′OMTase flow-through. The expected molecular weights of SNAP-T7 RNAP, SNAP-FCE, SNAP-2′OMTase are approximately 120 kDa, 120 kDa and 61 kDa, respectively.

FIGS. 50A-50F show in vitro mRNA capping efficiency of co-immobilized T7 RNAP, FCE and 2′OMTase by capillary electrophoresis. mRNA capping efficiency of serial 1:2 dilutions of T7 RNAP and FCE co-immobilization in benzylguanine-coated sera magnetic beads (FIG. 50A) and benzylguanine and PEG750-coated sera magnetic beads (FIG. 50B), FCE and 2′OMTase co-immobilization in benzylguanine-coated sera magnetic beads (FIG. 50C) and benzylguanine and PEG750-coated sera magnetic beads (FIG. 50D) and T7 RNAP, FCE and 2′OMTase co-immobilization in benzylguanine-coated sera magnetic beads (FIG. 50E) and benzylguanine and PEG750-coated sera magnetic beads (FIG. 50F). m7Gppp- is the final product (N7 methylase product), Gppp- is the product of the second enzyme activity (guanylyl transferase), p- is not a product of any capping reaction, pp- is the product of the first enzyme activity (triphosphatase) and ppp- is the substrate.

FIG. 51 shows in vitro mRNA Cap-1 capping efficiency of soluble 2′-OMTase and SNAP-2′-OMTase. Upper line and squares correspond to soluble 2′-OMTase, lower line and dots correspond to soluble SNAP-2′-OMTase.

FIG. 52 shows in vitro mRNA Cap-1 capping efficiency of soluble 2′-OMTase and immobilized SNAP-2′-OMTase. The y-axis indicates capping efficiency, Cap-0 and Cap-1 product are shown as indicated in the legend.

FIG. 53 shows in vitro mRNA Cap-0 capping efficiency at different reaction temperatures of soluble VCE and SNAP-FCE and immobilized SNAP-FCE in BG-, BG-PEG750 and BG-PEG4-PEG750 coated sera magnetic beads.

FIG. 54 shows an SDS-PAGE gel of fusion SNAP-FCE-T7 RNAP expression and purification in which the lanes contain the following: lane 1: crude extract, lane 2: soluble extract; lanes 3-4: flow-through of Ni column; lanes 5, 24: protein Standard (NEB, P7719S), lanes 6-23: elution fractions of Ni column. Solid-line box corresponds to SNAP-FCE-T7 RNAP molecular weight. The expected molecular weight of SNAP-FCE-T7 RNAP is approximately 220 kDa.

FIG. 55 shows an SDS-PAGE gel of SNAP-FCE-T7 RNAP immobilized on BG-PEG750-coated sera magnetic beads in which the lanes contain the following: lane 1: protein standard (NEB, P7719S); lane 2: SNAP-FCE-T7 RNAP load; lane 3: SNAP-FCE-T7 RNAP flow-through.

FIGS. 56A-56C show in vitro mRNA capping efficiency of soluble and immobilized fusion SNAP-FCE-T7 RNAP by capillary electrophoresis (CE). mRNA capping efficiency of serial 1:2 dilutions of SNAP-FCE-T7 RNAP in solution (FIG. 56A) and immobilized in benzylguanine-coated sera magnetic beads (FIG. 56B) and benzylguanine and PEG750-coated sera magnetic beads (FIG. 56C). m7Gppp- is the final product (N7 methylase product), Gppp- is the product of the second enzyme activity (guanylyl transferase), p- is a by-product of chemical synthesis of the RNA that is not reactive in capping reaction, pp- is the product of the first enzyme activity (triphosphatase) and ppp- is the substrate.

FIGS. 57A-57B show in vitro mRNA capping efficiency of co-immobilized FCE-T7 RNAP and 2′OMTase by capillary electrophoresis (CE). mRNA capping efficiency of serial 1:2 dilutions of FCE-T7 RNAP and 2′OMTase co-immobilization in benzylguanine-coated sera magnetic beads (FIG. 57A) and benzylguanine and PEG750-coated sera magnetic beads (FIG. 57B). m7Gppp- is the final product (N7 methylase product), Gppp- is the product of the second enzyme activity (guanylyl transferase), p- is a by-product of chemical synthesis of the RNA that is not reactive in capping reaction, pp- is the product of the first enzyme activity (triphosphatase) and ppp- is the substrate.

FIG. 58 shows in vitro transcription yield at three different temperatures using soluble SNAP-T7 RNAP and FCE at a 1:1 dilution, co-immobilized SNAP-T7 RNAP and SNAP-FCE in benzylguanine-coated sera magnetic beads at A 1:1 dilution, or a soluble SNAP-FCE-T7 RNAP fusion protein at a 1:1 dilution.

FIGS. 59A and 59B show example results of co-transcriptional capping by SNAP-T7 RNAP SNAP-FCE, SNAP-FCE-T7 RNAP and SNAP-2′OMT. FIG. 59A shows co-transcriptional capping efficiency of soluble SNAP-T7 RNAP and SNAP-FCE (Load T7+FCE), immobilized SNAP-T7 RNAP and SNAP-FCE in BG-magnetic beads (T7+FCE@BG) and in BG and PEG750-magnetic beads (T7+FCE@PEG); soluble SNAP-FCE-T7 RNAP (Load FCE-T7); immobilized SNAP-FCE-T7 RNAP in BG-magnetic beads (FCE-T7@BG) and in BG and PEG750-magnetic beads (FCE-T7@PEG). Left-Y axes and stacked bars indicates the capping efficiency, being m7Gppp- the final Cap 0 product. Right-Y axes and scattered circles indicate average RNA yield of the in vitro transcription and standard deviation of triplicates (in ng/μL), respectively. FIG. 59B shows co-transcriptional capping efficiency of soluble SNAP-T7 RNAP, SNAP-FCE and SNAP-2′OMTase (Load T7+FCE+2′OMTase), immobilized SNAP-T7 RNAP, SNAP-FCE and SNAP-2′-O-Methyltransferase in BG-magnetic beads (T7+FCE+2′OMTase@BG) and in BG and PEG750-magnetic beads (T7+FCE+2′OMTase@PEG); soluble SNAP-FCE-T7 RNAP and 2′-O-Methyltransferase (Load FCE-T7+2′OMTase); immobilized SNAP-FCE-T7 RNAP and 2′-O-Methyltransferase in BG-magnetic beads (FCE-T7+2′OMTase@BG) and in BG and PEG750-magnetic beads (FCE-T7+2′OMTase@PEG). Left-Y axes and stacked bars indicates the capping efficiency, with m7GpppGm- the final Cap 1 product. Right-Y axes and scattered circles indicate average RNA yield of the in vitro transcription and standard deviation of triplicates (in ng/μL), respectively.

### DETAILED DESCRIPTION

The present disclosure relates, in some embodiments, to an immobilized enzyme comprising a support and an enzyme immobilized thereto. According to some embodiments, a linker may be disposed between the enzyme and the support. An immobilized enzyme may comprise a type IIS restriction endonuclease with a recognition sequence and cleave site of 5′ GCTCTTC N1/N4 3′ (e.g., BspQI; positions 33-39 of SEQ ID NOS: 13 and 14), a support (e.g., a magnetic bead), and a linker (e.g., a peptide linker). In some embodiments, an immobilized enzyme may comprise a ligand (e.g., O⁶-benzyleguanine) and a receptor or tag (e.g., a SNAP-tag®) capable of binding the ligand. For example, ligands may be disposed on a support and corresponding receptors may be disposed on (e.g., covalently attached to) an enzyme to be immobilized on the support. An immobilized enzyme may comprise, in some embodiments, an enzyme (e.g., BspQI, Nt.BspQI, T7 RNAP, FCE, 2′OMT), optionally, a first linker (e.g., a peptide linker) attached to the enzyme, a polypeptide tag (e.g., a SNAP-tag®) attached to the first linker, if present, or the enzyme, a ligand corresponding to the polypeptide tag (e.g., O⁶-benzyleguanine) attached (e.g., covalently attached) to the tag, optionally, a second linker (e.g., polyethylene glycol) attached to the ligand, and a support (e.g., a magnetic bead) attached to the second linker if present or the ligand, the structure of which may be illustrated as:
ENZYME-[LINKER-]TAG-LIGAND-[LINKER-]SUPPORT,
   wherein dashes represent bonds (covalent or non-covalent) and brackets represent optional elements. For example, an immobilized BspQI may comprise BspQI, a glycine-serine peptide linker (e.g., GSx6) attached to the BspQI (e.g., by a peptide bond), a SNAP-tag attached to the linker (e.g., by a peptide bond), O⁶-benzyleguanine attached to the SNAP-tag, and magnetic beads attached to the O⁶-benzyleguanine. An immobilized RNAP may comprise RNAP, a glycine-serine peptide linker (e.g., GSx6) attached to the RNAP, (e.g., by a peptide bond), a SNAP-tag attached to the linker (e.g., by a peptide bond), O⁶-benzyleguanine attached to the SNAP-tag, and magnetic beads attached to the O⁶-benzyleguanine. An immobilized FCE may comprise FCE, a glycine-serine peptide linker (e.g., GSx6) attached to the FCE (e.g., by a peptide bond), a SNAP-tag attached to the linker (e.g., by a peptide bond), O⁶-benzyleguanine attached to the SNAP-tag, and magnetic beads attached to the O⁶-benzyleguanine. An immobilized 2′OMT may comprise 2′OMT, a glycine-serine peptide linker (e.g., GSx6) attached to the 2′OMT (e.g., by a peptide bond), a SNAP-tag attached to the linker (e.g., by a peptide bond), O⁶-benzyleguanine attached to the SNAP-tag, and magnetic beads attached to the O⁶-benzyleguanine.

According to some embodiments, an immobilized enzyme may comprise a support, a first enzyme (e.g., a type IIS restriction endonuclease, an RNAP, a capping enzyme) attached to the support, and a second enzyme (e.g., a second restriction endonuclease, an exonuclease, a polymerase, or a capping enzyme, in each case different from the first enzyme) attached to the support. In some embodiments, an immobilized enzyme composition may comprise a first support, a first enzyme (e.g., a type IIS restriction endonuclease, an RNAP, a capping enzyme) attached to the support, a second support and a second enzyme (e.g., a second restriction endonuclease, an exonuclease, a polymerase, or a capping enzyme, in each case different from the first enzyme) attached to the second support.

The present disclosure relates to methods comprising contacting an immobilized enzyme to a substrate to form a product, separating the immobilized enzymes from the product, and optionally contacting the immobilized enzyme (e.g., a fresh aliquot of the immobilized enzyme, the separated enzyme, or both) to more substrate to form more product. For example, a method may comprise contacting a first portion of a double stranded DNA substrate with an immobilized enzyme comprising a type IIS restriction endonuclease (e.g., BspQI) to produce double stranded DNA cleavage products, separating the immobilized enzyme from the double stranded DNA cleavage products to form separated immobilized enzyme and separated double stranded DNA cleavage products, and/or contacting a second portion of the double stranded DNA substrate with the separated immobilized enzyme comprising a type IIS restriction endonuclease to produce more double stranded DNA cleavage products. In some embodiments, a method may further comprise repeating the contacting and separating steps (e.g., 2-50 cycles). A method may further comprise combining the separated double stranded DNA cleavage products with the more double stranded DNA cleavage products to produce pooled products. According to some embodiments, methods may include contacting substrates and/or products with an additional enzyme (e.g., an additional immobilized enzyme). Contacting with an additional enzyme may comprise, in some embodiments, contacting a substrate precursor with the additional enzyme to produce initial reaction products comprising a substrate (e.g., a double-stranded polynucleotide substrate). In some embodiments, contacting with an additional enzyme may comprise, following contact with an immobilized enzyme to form reaction products, contacting the reaction products with the additional enzyme to produce further reaction products.

Methods may include, in some embodiments, forming a polyribonucleotide (e.g., mRNA) from a polynucleotide template (e.g., a double stranded DNA template) by contacting the template (e.g., a nicked double stranded DNA template) with a polynucleotide polymerase (e.g., an RNA polymerase). A method may further include providing a template ready for use in an in vitro transcription reaction or preparing the template. Preparing a template may comprise contacting a double stranded DNA polynucleotide comprising the template with an immobilized enzyme (e.g., BspQI) to form a nicked polynucleotide template. In some embodiments, a method may comprise contacting a double stranded DNA polynucleotide comprising the template with an immobilized enzyme to form a nicked polynucleotide template, contacting the nicked polynucleotide template with an RNA polymerase (e.g., T7 RNA polymerase, SP6 polymerase, T3 and variants thereof) to form the polyribonucleotide (e.g., a transcription product having sequence complementary to the polynucleotide template), wherein the immobilized enzyme comprises a type IIS restriction endonuclease, a support, and a linker disposed between the type IIS restriction endonuclease and the support. According to some embodiments, an RNA polymerase may be a soluble RNA polymerase or an immobilized RNA polymerase. An RNA polymerase may be immobilized on a support separate from the support on which the type IIS restriction endonuclease is immobilized or the RNA polymerase and the type IIS restriction endonuclease may be co-immobilized on a common support. A polynucleotide template may comprise, for example, in a 5′-3′ direction, a coding sequence, a poly(U) sequence, and a type IIS restriction endonuclease recognition sequence. A poly(U) sequence, in some embodiments, may be up to 100 nucleotides in length, up to 200 nucleotides in length, up to 300 nucleotides in length, up to 400 nucleotides in length, up to 500 nucleotides in length, or more than 400 nucleotides in length. RNAs, including RNAs (e.g., mRNAs) with long poly(A) sequences, may be useful in transient gene expression and/or therapeutic applications (Grier et al., Molecular Therapy—Nucleic Acids (2016) 5, e306).

A method may comprise, in some embodiments, capping an RNA (e.g., an mRNA) by contacting the RNA and a capping enzyme. Example capping enzymes may include vaccinia capping enzyme, Faustovirus capping enzyme, and 2′ O-methyltransferase (2′OMT or 2′OMTase). Example capping enzyme forms may include a soluble capping enzyme, a soluble fusion enzyme (e.g., a soluble polypeptide comprising the capping enzyme and another enzymatic function, such as an RNA polymerase), an immobilized capping enzyme, and an immobilized fusion enzyme (e.g., an immobilized polypeptide comprising the capping enzyme and another enzymatic function, such as an RNA polymerase).

According to some embodiments, a method may comprise one or more of (a) contacting a double stranded DNA template with a type IIS restriction endonuclease (e.g., BspQI) to form a nicked template, (b) contacting a nicked template with an RNA polymerase (e.g., T7 RNA polymerase) to form an RNA polynucleotide, (c) contacting an RNA polynucleotide with a Cap-0 capping enzyme (e.g., vaccinia capping enzyme or Faustovirus capping enzyme) to form a Cap-0 RNA polynucleotide, and (d) contacting a Cap-0 RNA polynucleotide with a Cap-1 capping enzyme (e.g., 2′OMT) to form a Cap-1 RNA polynucleotide, wherein the type IIS restriction endonuclease, the RNA polymerase, the Cap-0 enzyme, and the Cap-1 each enzyme independently may be a soluble enzyme or an immobilized enzyme and/or wherein each enzyme independently may be a member of a fusion protein (e.g., with one of the other enzymes).

The present disclosure also relates to methods of making an immobilized enzyme. Methods may comprise, for example, contacting an enzyme (e.g., a type IIS restriction endonuclease, an RNAP, a capping enzyme) with a support (e.g., magnetic bead) to form an immobilized enzyme (e.g., an immobilized type IIS restriction endonuclease, an immobilized RNAP, an immobilized capping enzyme). An enzyme for immobilization may include a wild-type enzyme and/or a non-naturally occurring enzyme. Enzymes may comprise one or more tags (e.g., expression, purification tags, ligand-binding tags), secretion signals, linkers, and/or post-translational modifications, each of which may be independently located at the N-terminal end, the C-terminal end, or anywhere along the length of the enzyme. For example, an enzyme for immobilization may include, in an N-terminal to C-terminal direction, a histidine purification tag, a ligand-binding tag, a linker, and an enzyme.

The present disclosure generally relates to methods and compositions for preparing polynucleotide libraries, for example, as disclosed in International PCT Application No. PCT/US2020/050520 filed Sep. 11, 2020, and incorporated herein by reference. Polynucleotide libraries, in some embodiments, may be prepared for sequencing using the disclosed methods and compositions. In some embodiments, compositions comprising polynucleotides (e.g., fragments) may be subjected to coupled reactions in which soluble enzymes, immobilized enzymes, or both soluble and immobilized enzymes repair or condition the ends of the polynucleotides, tail one or both ends, and/or ligate the polynucleotides to a sequencing adapter. One or more of the enzymes used may be immobilized on a bead (e.g., a magnetic bead) or other solid support. For example, in a coupled reaction comprising a tailing reaction and a ligation reaction, a tailing enzyme and a ligase may be immobilized on separate supports or co-immobilized on a common support. Immobilized enzymes may reduce or obviate the need for damaging bead purification steps. Bead purification may be used to remove soluble enzymes and other compounds in the reaction media, but may also damage the polynucleotides being purified and may introduce contaminating chemicals present on the beads or in required wash solutions (e.g., ethanol and PEG among others) that may interfere with subsequent uses of the purified polynucleotides (e.g., sequencing). Library preparation methods using immobilized enzymes may require lower amounts of input polynucleotides to achieve the same number of sequencing reads and may better preserve the activity of transmembrane pores used in sequencing. Library preparation and sequencing workflows using immobilized enzymes may be automated and may include reuse of immobilized enzymes, preserving reagents and lowering costs.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible. Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements. Lists of example species within a particular genus may vary in length at different places throughout the disclosure. Species lists shortened for convenience shall not be construed to exclude example species listed elsewhere in the specification.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins. It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “adapter” refers to a sequence that is joined to or can be joined to another molecule (e.g., ligated or copied onto via primer extension). An adapter can be DNA or RNA, or a mixture of the two. An adapter may be 15 to 100 bases, e.g., 50 to 70 bases, although adapters outside of this range are envisioned. In a library of polynucleotide molecules that contain an adapter (e.g., a 3′ or 5′ adapter, the adapter sequence used is not present in the DNA sequences under examination (i.e., the sequence in between the adapters). For example, if the library of polynucleotide molecules contains sequences derived from mammalian genomic DNA, cDNA or RNA, then the sequences of the adapters are not present in the mammalian genome under study. In many cases, the 5′ and 3′ adapters are of a different sequence and are not complementary. In many cases, an adapter will not contain a contiguous sequence of at least 8, 10 or 12 nucleotides that is found in the DNA under examination. Adapters may be designed to serve a specific purpose. For example, adapters may be designed for use in sequencing applications. Sequencing adapters may comprise, for example, an oligo-(dT) overhang, a barcode sequence, an overhang (other than oligo-(dT)) to anneal to another adapter, a site for anchoring a motor protein, and a sequence to bind to tethering oligos with affinity to polymer membrane for guiding a DNA or RNA fragment (on which it resides) to the vicinity of a nanopore, and combinations thereof.

In the context of the present disclosure, “adapter-containing” refers to either a nucleic acid that has been ligated to an adapter, or to a nucleic acid to which an adapter has been added by primer extension. In some embodiments, the adapters of a library of nucleic acid molecules may be made by ligating oligonucleotides to the 5′ and 3′ ends of the molecules (or specific sequences of the same) in an initial nucleic acid sample, e.g., DNA or genomic DNA, cDNA.

In the context of the present disclosure, “bead purification” refers to use of magnetic beads to preferentially adsorb polynucleotide molecules (e.g., RNA, DNA) away from soluble enzymes (and optionally, other components) through a series of binding, washing, and elution steps.

In the context of the present disclosure, “buffer” or “buffering agent” refers to an agent that, when in solution or in contact with a solution, contributes to our causes such solution to resist changes in pH upon addition of acid(s) or alkali(s) to the solution. Examples of suitable non-naturally occurring buffering agents that may be used include, for example, any of Tris, HEPES, TAPS, MOPS, tricine, and MES.

In the context of the present disclosure, “capping” refers to the enzymatic addition of a Nppp-moiety onto the 5′ end of an RNA, where N a nucleotide such as is G or a modified G. A modified G may have a methyl group at the N7 position of the guanine ring, or an added label at the 2 or 3 position of the ribose, and, in some embodiments, the label may be an oligonucleotide, a detectable label such as a fluorophore, or a capture moiety such as biotin or desthiobiotin, where the label may be optionally linked to the ribose of the nucleotide by a linker, for example. See, e.g., WO 2015/085142. A cap may have a Cap-0 structure, a Cap-1 structure or a cap 2 structure (as reviewed in Ramanathan, Nucleic Acids Res. 2016 44: 7511-7526), depending on which enzymes and/or whether SAM is present in the capping reaction.

In the context of the present disclosure, “capping efficiency” refers to the proportion of RNA transcription products that have a cap structure (e.g., Cap-0, Cap-1, or Cap-2) as measured by any available assay including, for example, the assay described in Example 38. Capping efficiency is calculated as the intensity of capped peaks (e.g., Cap-0, Cap-1, Cap-2, or combinations thereof) divided by the sum of the intensity of capped peaks (e.g., Cap-0, Cap-1, Cap-2, or combinations thereof) and the intensity of the 5′ppp peaks). Methods and compositions (e.g., compositions comprising immobilized enzyme, a co-immobilized enzyme and/or a coupled enzyme) may have a capping efficiency of at least 35%, at least 40%, at least 45%, at least 50%, at least 55%, at least 60%, at least 65%, at least 70%, at least 75%, at least 80%, at least 85%, at least 90%, at least 95%, or as much as 100%.

In the context of the present disclosure, “coupled reaction” refers to a reaction in which two or more reaction steps occur in a single reaction mixture and in a single reaction vessel (e.g., a tube, a well, a capillary, a flow cell, a surface). Sequential reaction steps in a coupled reaction may begin and/or continue without changes to reaction conditions (e.g., without addition or removal of reagents, changes in temperature, pH, volume, or washing) beyond those that arise or follow from the reactions themselves. For example, a coupled reaction may include a reaction in which a polymerase (e.g., an immobilized polymerase) is combined in a single reaction vessel with a ligase (e.g., an immobilized ligase) and both tailing and ligation reactions proceed in the same mixture (e.g., without an intervening bead purification). For clarity, coupled reactions include reactions in which microenvironments may exist (e.g., on the surface of individual microbeads in the reaction mixture).

In the context of the present disclosure, “Faustovirus RNA capping enzyme” refers to a single-chain RNA capping enzyme capable of capping RNA (e.g., having detectable TPase, GTase and N7 MTase activity) including, for example, Faustovirus enzymes described in US20210054016, US20210395706, and U.S. Ser. No. 11/028,379.

In the context of the present disclosure, “fragment” refers to a polynucleotide. A fragment may originate from in vitro or in vivo synthetic processes. A population of fragments may include full-length polynucleotides (as originally synthesized) and/or smaller portions of such full-length sequences resulting from mechanical, chemical, and/or enzymatic breakage.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a reporter protein. Alternatively, a fusion may comprise a non-naturally occurring combined polypeptide chain comprising two proteins or two protein domains joined directly to each other by a peptide bond or joined through a peptide linker.

In the context of the present disclosure, “immobilized” refers to covalent attachment of an enzyme to a solid support with or without a linker. Examples of solid supports include beads (e.g., magnetic, agarose, polystyrene, polyacrylamide, chitin). Beads may include one or more surface modifications (e.g., O⁶-benzyleguanine, polyethylene glycol) that facilitate covalent attachment and/or activity of an enzyme of interest. For example, a support may comprise a ligand and an enzyme may have a receptor for such ligand or an enzyme may comprise a ligand and a support may comprise a receptor for such ligand. Receptor-ligand binding may be covalent or non-covalent. Non-covalent attachment (e.g., avidin:biotin, chitin:CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand-receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “in vitro transcription” (IVT) refers to a cell-free reaction in which a double-stranded DNA (dsDNA) template is copied by a DNA-directed RNA polymerase (typically a bacteriophage polymerase) to produce a product that contains RNA molecules copied from the template.

In the context of the present disclosure, “library” or “polynucleotide library” refers to a mixture of different molecules. A library may comprise DNA and/or RNA (e.g., genomic DNA, organelle DNA, cDNA, mRNA, microRNA, long non-coding RNAs or other RNAs of interest) or fragments thereof from any desired source (e.g., human, non-human mammal, plant, microbe, virus, or synthetic). A library may have any desired number of different polynucleotides. For example, a library may have more than 10⁴, 10⁵, 10⁶ or 10⁷ different nucleic acid molecules. A library may have fewer different molecules, for example, where the molecules collectively have more than 10⁴, 10⁵, 10⁶ or 10⁷ or more nucleotides. In some embodiments, a library of polynucleotide molecules may be an enriched library, in which case the library may have a complexity of less than 10%, less than 5%, less than 1%, less than 0.5%, or less than 0.1%, less than 0.01%, less than 0.001% or less than 0.0001% relative to the unenriched sample (e.g., a sample made from total RNA or total genomic DNA from a eukaryotic cell sample. Molecules can be enriched by methods such as described in US2014/0287468 or US 2015/0119261. A library, in some embodiments, may include member polynucleotides that are tagged with an adapter.

In the context of the present disclosure, “ligase” refers to enzymes that join polynucleotide ends together. Ligases include ATP-dependent double-strand polynucleotide ligases, NAD+-dependent double-strand DNA or RNA ligases and single-strand polynucleotide ligases. Ligases may include any of the ligases described in EC 6.5.1.1 (ATP-dependent ligases), EC 6.5.1.2 (NAD+-dependent ligases), EC 6.5.1.3 (RNA ligases) (see ExPASy Bioinformatics Resource Portal having a URL of enzyme.expasy.org which is a repository of information concerning nomenclature of enzymes based on the recommendations of the Nomenclature Committee of the International Union of Biochemistry and Molecular Biology (IUBMB) describing each type of characterized enzyme for which an EC (Enzyme Commission) number has been provided. Specific examples of ligases include bacterial ligases such as E. coli DNA ligase and Taq DNA ligase, Ampligase® thermostable DNA ligase (Epicentre® Technologies Corp., part of Illumina®, Madison, Wis.) and phage ligases such as T3 DNA ligase, T4 DNA ligase, T7 DNA ligase, 9° N DNA ligase, and mutants thereof. In some embodiments, a ligase may be included in a fusion protein with a SNAP-tag protein.

In the context of the present disclosure, “magnetic bead” refers to a support comprising a surface and a core. A surface may comprise one or more surface modifications including polyethylene glycol (e.g., O⁶-benzyleguanine and/or PEG₇₅₀). A magnetic core may comprise one or more magnetic or magnetizable materials including, for example, iron, an iron oxide, cobalt a cobalt oxide, nickel, a nickel oxide, or combinations thereof.

In the context of the present disclosure, “magnetically gathering” refers to application of a magnetic field to a subject surface or container. A magnetic field may be applied by forming a magnetic field at or near a surface or container, or by bringing a surface or container into the effective range of an existing magnetic field, for example, by moving the surface or container near the existing field and/or by reshaping a field. Magnetically gathering immobilized enzymes into a group may include forming a pellet of immobilized enzyme. Such pellet may be sufficiently well formed and stable to tolerate manipulation or removal of a fluid, composition, or reaction mixture adjoining and/or in contact with the pellet.

In the context of the present disclosure, “modified nucleotides” (including references to modified NTP, modified ATP, modified GTP, modified CTP, and modified UTP) refers to any noncanonical nucleoside, nucleotide or corresponding phosphorylated versions thereof. Modified nucleotides may include one or more backbone or base modifications. Examples of modified nucleotides include dI, dU, 8-oxo-dG, dX, and THF. Additional examples of modified nucleotides include the modified nucleotides disclosed in U.S. Patent Publication Nos. US20170056528A1, US20160038612A1, US2015/0167017A1, and US20200040026A1. Modified nucleotides may include naturally or non-naturally occurring nucleotides.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., buffering agents, a detergent, a dye, a solvent or a preservative).

In the context of the present disclosure, “polymerase” refers to enzymes that catalyze the formation of polynucleotides (e.g., DNA, RNA) from nucleoside triphosphates in a 5′-3′ direction. Addition of nucleoside triphosphates may be templated or untemplated. Polymerases include DNA polymerases classified into families A, B, C, D, X, Y and RT. Polymerases include RNA polymerases classified into families I, II, III, IV, and V. Examples of polymerases include Q5® polymerases, Taq polymerases, Bst polymerases, Bsu polyerases, phi29 polymerases, T4 DNA polymerase, T7 DNA polymerase, DNA pol I, Therminator™, Klenow, Vent® polymerases, Deep Vent® polymerases, terminal transferase, 9° N polymerase, T3 RNA polymerase, T7 RNA polymerases, SP6 polymerases, polyA polymerases, and polyU polymerases.

In the context of the present disclosure, “type IIS restriction endonuclease” refers to enzymes that recognize asymmetric DNA sequences and cleave outside of their recognition sequence. Type IIS restriction endonucleases may have contains two catalytic sites in a single polypeptide chain. Examples of such enzymes include BspQI, Nt.BspQI, and Nt.BstNBI. Type IIS restriction endonucleases may include those commercially available, for example, from New England Biolabs, Inc. (Tools and Resources, Type IIS Restriction Enzyme Chart incorporated by reference).

In the context of the present disclosure, “tailing enzyme” refers to template-independent enzymes (e.g., polymerases, transferases) that add one or more nucleotides or ribonucleotides to the 3′ end of a polynucleotide. Tailing enzymes may add one or more As, one or more Gs, one or more Ts, one or more Cs, or one or more Us. Tailing enzymes may be selected for specific applications based on their preference for adding a particular nucleotide or ribonucleotide, for example, to compliment the end of an adapter to which the tailed polynucleotide. Examples of tailing enzymes include poly(A) polymerases, poly(G) polymerases, poly(U) polymerases, and terminal deoxynucleotidyl transferase (TdT). In some embodiments, a tailing enzyme may be included in a fusion protein with a SNAP-tag protein.

In the context of the present disclosure, “transmembrane pore” refers to protein pores and solid state pores. A transmembrane pore may be a nanopore. Transmembrane protein pores may be or comprise hemolysin, leucocidin, lysenin, a Mycobacterium smegmatis porin (e.g., MspA, MspB, MspC, MspD), CsgG, an outer membrane porin (e.g., OmpF, OmpG), outer membrane phospholipase A, Neisseria autotransporter lipoprotein (NalP), WZA, or variants thereof. In the context of the present disclosure, “unique molecule identifier” (UMI) refers to a random unique sequence of at least 6 nucleotides (6N). Longer random unique sequences may be used, for example, 2-15 nucleotides, 6-12 nucleotides, or 8-12 nucleotides. UMIs may have sufficient sequence diversity to distinguish the molecule of which they are a part (e.g., an adapter or a tagged fragment) from other molecules in a mixture.

In the context of the present disclosure, “uncapped” refers to an RNA (a) that does not have a cap and (b) that can be used as a substrate for a capping enzyme. Uncapped RNA typically has a tri- or di-phosphorylated 5′ end. RNAs transcribed in vitro have a triphosphate group at the 5′ end.

All publications (including all co-published supplemental and supporting information), patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, Mass.).

Methods

The present disclosure provides methods for preparing a library for sequencing. For example, a method may comprise (a) in a coupled reaction, (i) contacting a population of nucleic acid fragments with a tailing enzyme to produce tailed fragments, and (ii) ligating to the tailed fragments a sequencing adapter with a ligase to produce adapter-tagged fragments; and/or separating adapter-tagged fragments from the tailing enzyme and the ligase to produce separated adapter-tagged fragments and, optionally, separated tailing enzyme and/or separated ligase. In some embodiments, a tailing enzyme and/or a ligase used in library preparation may be immobilized enzymes. For example, a tailing enzyme may be immobilized on a magnetic bead and/or a ligase may be immobilized on a magnetic bead. Optionally, a tailing enzyme and a ligase may be immobilized on the separate supports or co-immobilized on a single support. A tailing enzyme and/or a ligase, according to some embodiments, may be soluble enzymes. In some embodiments, separating adapter tagged fragments may further comprise subjecting the coupled reaction to a magnetic field (e.g., bringing the sample to a magnet, bringing a magnet to the sample, activating an electromagnetic field). A population of nucleic acid fragments may comprise ribonucleic acid fragments and/or may comprise deoxyribonucleic acid fragments. In some embodiments, methods may be capable of producing sequencing libraries with little input RNA. For example, methods may use a population of nucleic acid fragments having less than 100 ng of nucleic acids or a population of nucleic acid fragments having less than 10 ng of nucleic acids.

The present disclosure further provides methods for preparing sequencing libraries comprise any combination of steps (a) and (b) and further comprise: (c) in a second coupled reaction, (i) contacting a second population of nucleic acid fragments with the separated tailing enzyme to produce additional tailed fragments, and (ii) ligating to the additional tailed fragments a second sequencing adapter with the separated ligase to produce additional adapter-tagged fragments, and/or (d) separating the additional adapter-tagged fragments from the separated tailing enzyme and the separated ligase to produce separated additional adapter-tagged fragments, separated tailing enzyme, and separated ligase. In some embodiments, a method comprise any combination of steps (a), (b), (c), and (d) and may further comprise (e) translocating the separated adapter-tagged fragments through one or more transmembrane pores; (0 detecting electrical changes as the one or more separated adapter-tagged fragments are translocated through the one or more transmembrane pores in an insulating membrane to produce an electrical signal; and/or (g) analyzing the electrical signal to generate a sequence read. In some embodiments, one or more transmembrane pores may retain about 90% of their initial activity after two hours and/or may retain about 50% of their initial activity after 8 hours. One or more transmembrane pores, according to some embodiments of the disclosure, may produce at least 900 sequence reads per transmembrane pore. In some embodiments, a sequencing adapter may be a single-stranded adapter and may comprise a leader sequence; and a first sequence and a second sequence, wherein the first and second sequences are complementary to each other and define a hairpin, wherein the leader sequence is configured to thread into the one or more transmembrane pores.

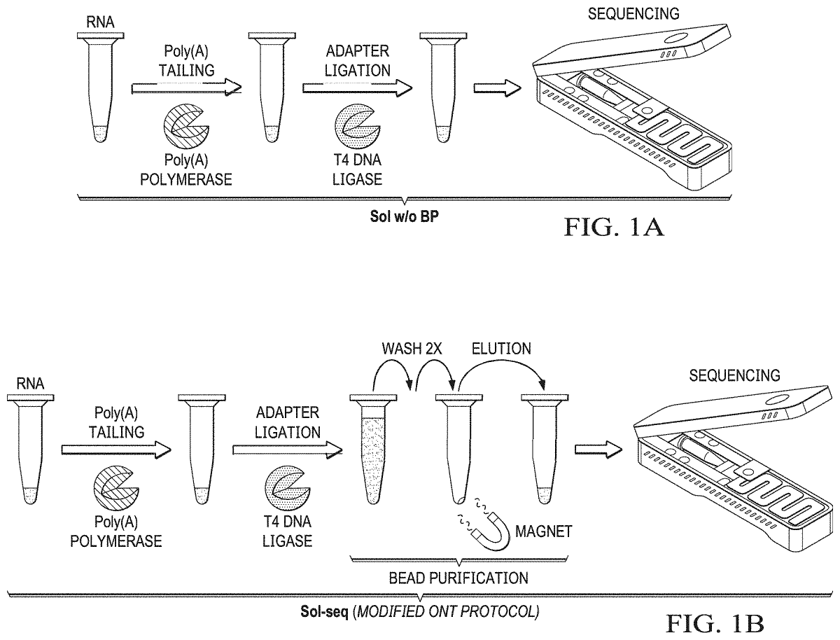

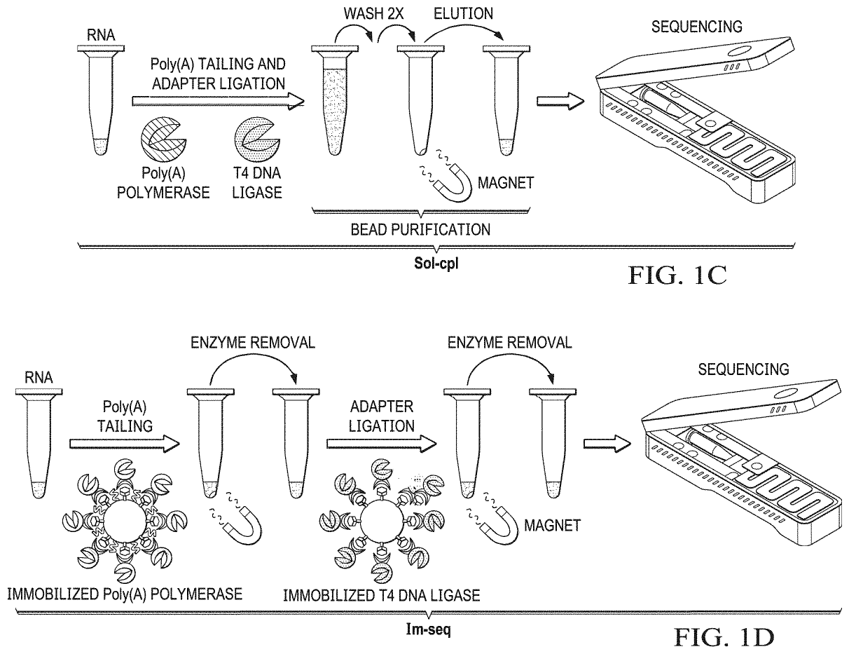

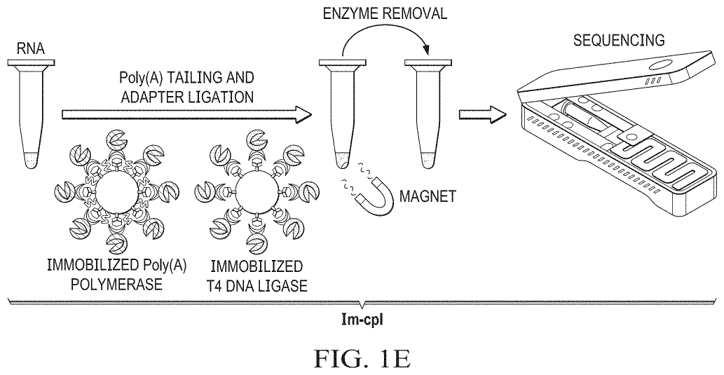

FIGS. 1A-1E illustrate some embodiments of methods and compositions disclosed herein. For example, a method may include sequentially contacting an RNA composition (e.g., comprising one or more species of RNA molecules) and a soluble tailing enzyme (optionally in the presence of a buffer) to produce a polyA tailed RNA composition, contacting the polyA tailed RNA composition with an adapter and a soluble ligase to produce ligated products, washing the ligated products, eluting the ligated products, and sequencing the ligated products (FIG. 1A).

A coupled method may include contacting an RNA composition (e.g., comprising one or more species of RNA molecules), a soluble tailing enzyme, an adapter, and a soluble ligase (optionally in the presence of a buffer) to produce ligated products, washing the ligated products, eluting the ligated products, and sequencing the ligated products (FIG. 1B). A coupled method may include contacting a linear DNA template, a soluble or immobilized RNA polymerase, a soluble or immobilized capping enzyme, and optionally one or more of NTPs, modified NTPs, S-adenosyl methionine (SAM), and a buffering agent, optionally in the range of 42° C.-47° C., e.g., 44° C.-46° C. to efficiently produce capped RNA.

A method, in some embodiments, may include sequentially contacting an RNA composition (e.g., comprising one or more species of RNA molecules) and a soluble tailing enzyme (optionally in the presence of a buffer) to produce a polyA tailed RNA composition, contacting the polyA tailed RNA composition with an adapter and a soluble ligase to produce ligated products, and directly (e.g., without washing or elution) sequencing the ligated products (FIG. 1C).

In some embodiments, a method may include sequentially contacting an RNA composition (e.g., comprising one or more species of RNA molecules) and an immobilized tailing enzyme (optionally in the presence of a buffer) to produce a polyA tailed RNA composition, removing the immobilized tailing enzyme (e.g., in the case of enzymes bound to magnetic beads, magnetically gathering the magnetic beads into a group and taking away the polyA tailed composition, for example, by pipetting away from the beads), contacting the polyA tailed RNA composition with an adapter and an immobilized ligase to produce ligated products, removing the immobilized ligase (e.g., in the case of enzymes bound to magnetic beads, magnetically gathering the magnetic beads into a group and taking away the ligated products, for example, by pipetting away from the beads), and directly (e.g., without further washing or elution) sequencing the ligated products (FIG. 1D).

A coupled method, in some embodiments, may include contacting an RNA composition (e.g., comprising one or more species of RNA molecules), an immobilized tailing enzyme, an adapter, and an immobilized ligase (optionally in the presence of a buffer) to produce ligated products, removing the immobilized tailing enzyme and the immobilized ligase (e.g., in the case of enzymes bound to magnetic beads, magnetically gathering the magnetic beads into a group and taking away the ligated products, for example, by pipetting away from the beads), and directly (e.g., without further washing or elution) sequencing the ligated products (FIG. 1E).

With respect to its corresponding soluble enzyme, an immobilized enzyme is physically constrained to a support which defines a microenvironment for the immobilized enzyme molecules and its substrates. This microenvironment may differ from a corresponding soluble enzyme with potential implications on the stability and/or activity of the immobilized enzyme. Microenvironmental differences may impact conformation of the enzyme, flexibility of the enzyme (e.g., to undergo changes required by or associated with its catalytic mechanism), the relationship between the enzyme and the milieu (e.g., solvent), accessibility of the active site to substrate, accessibility of binding sites for cofactors and/or coenzymes among others. Within these microenvironments, surface environments (e.g. charges, functional groups, morphology, hydrophilicity) of the support materials can also effect the enzymatic rate and immobilized enzyme stability. Therefore, improving and optimizing this microenvironment may enhance or maximize enzymatic activities upon immobilization. One or more strategies to alter surface microenvironment may be used to improve activity of immobilized enzymes. A single optimization solution may not be applicable to all enzymes. In some embodiments, various blocking groups or bead coatings (ethanolamine and polyethylene glycol—PEG of different lengths) can be utilized to modify hydrophilicity of support surface. For example, polyethylene glycol (PEG) moieties can be used to modify the surface of BG-functionalized magnetic beads. This PEG coating strategy has been shown to be effective in enhancing activity of several enzymes validated, including T4 DNA polymerase, Taq DNA polymerase and T4 DNA ligase) (Li et al. 2018). According to some embodiments, the distance between the immobilized enzyme and the beads surface may play a key role in retaining or reducing enzymatic activity. By using the proper conjugation chemistry, polyethylene glycol (PEG) linkage groups with variable length can be applied as a spacer in between a SNAP-reactive BG and the bead surface. Various benzylguanine (BG) moieties (with PEGlated or non-PEGylated linkers) may confer different spatial arrangement of conjugated enzyme molecules. In some embodiments, solid phase catalysis strategically considers the substrate properties and accessibility which can be affected by surface properties and enzyme orientation. In addition, CLIP-reactive benzylcytosine (BC) moieties can be utilized to substitute for BG moieties on solid support because BC moieties are considered to be more hydrophilic than BG moieties. With this strategy, a target enzyme is fused to CLIP-tag instead of SNAP-tag. According to some embodiments, a bio-orthogonal conjugation strategy can simultaneously co-immobilize two enzymes in a desired molar ratio onto beads functionalized with SANP-reactive BG and CLIP-reactive BC moieties. Selection of support materials and proper modifications may enhance enzymatic activity and thermostability. The surface properties can modulate refolding upon relaxation and denaturation of enzyme globular structures thereby maintaining or regaining activity after storage and heat treatment.

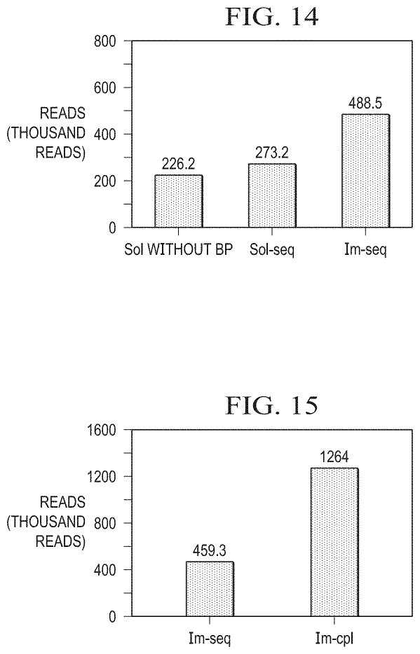

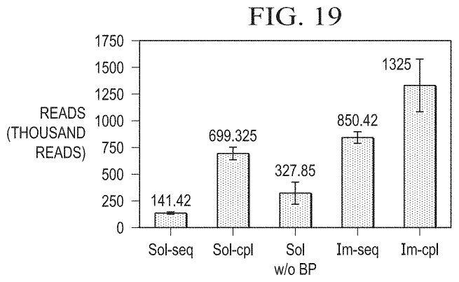

In RNA sequencing reactions or other applications using nanopores, the nanopores may be clogged, inactivated, and/or otherwise compromised by proteins that may be present in the compositions contacted with the nanopores. Accordingly, methods, applications, protocols and workflows including nanopores may comprise removing proteins (e.g., soluble proteins) by bead purification to alleviate such fouling. In some embodiments, the need for bead purification may be reduced or obviated by optimizing enzymatic reactions, for example, by reducing the amounts of enzymes used (e.g., effectively decreasing the ratio of enzyme to product in a reaction). Reducing the amount of enzyme(s) may reduce nanopore fouling thereby extending the functional time of nanopores in flow cells. While a reduction in the quantity of enzymes used may apply to all proteins or all enzymes in a reaction, since each protein and enzyme may interact with a given nanopore differently, reductions may be made on a more selective basis, targeting those that are more prone to fouling. As explained in more detail in the Example section below, FIG. 14 & FIG. 19 show that it is possible to generate sequence reads from libraries without bead purification, confirming that optimization of reactions with soluble enzymes enhance library preparation and/or performance.

Current ONT protocol uses 3 ul of T4 DNA Ligase (NEB M202M 2000 units/ul) or 6000 units for 500 ng input library. Use of immobilized enzymes and/or coupled reactions may reduce the amount of soluble T4 DNA Ligase by 90% (i.e. use of 600 units of ligase) because the immobilized enzyme protocols validated used only 180 units. For low input RNA library (<100 ng input), enzyme consumption can be further lowered.

Enzyme immobilization may provide opportunities to enhance performance of enzymatic processes, for example, by allowing faster and more efficient production of products, at least in part, by reducing or eliminating purification steps needed for corresponding soluble enzyme processes, by reducing reactant and/or product losses from washing steps, and/or by allowing enzymes to be reused in subsequent reaction cycles. Immobilization may imbue bound enzymes with additional thermostability and/or thermoactivity. For example, immobilized enzymes may tolerate higher temperatures (even if they are not catalytically active at such higher temperatures), which could be useful for applications in which enzymes are reused. In some embodiments, enzyme immobilization may allow soluble enzyme processes to be automated (or automated more efficiently). Immobilization may also allow processes to be more effective and/or efficient by reducing enzyme carry over to subsequent steps.

In some embodiments, methods including immobilized enzymes may omit or exclude heat treatments to inactivate enzymes, bead purification steps, and/or sequencing pore clogging. Heat stress can lead to the accumulation of 8-oxoguanine, deaminated cytosine, and apurinic DNA sites (AP-sites) in a cell (Bruskov V. I., Malakhova L. V., Masalimov Z. K., Chernikov A. V./Nucleic Acids Res. 2002. V. 30. P. 1354-1363.19. Lindahl T., Nyberg B./Biochemistry. 1974. V. 13. P. 3405-3410.20. Wailers R. L., Brizgys L. M./J. Cell Physiol. 1987. V. 133. P. 144-150. Elimination of bead purification may result in more uniformly sized fragments in a library to be sequenced. Bead purification may result in alteration of a library such as size distribution; For example, large or small species may be lost more than the species in the middle size range due to either less binding (leading to more loss) or tighter binding resulting in lower elution efficiencies. This step may also introduce impurities (present in loading and wash solutions) that may affect performance or parameters of nanopores such as signals or functioning time.

According to some embodiments, methods including immobilized enzymes may be adapted to and performed in microfluidic, lab-on-a-chip formats with enzymes immobilized on surfaces. For example, systems for single-cell RNA sequencing that produce RNA of a single cell may be adapted to contact such RNA with a tailing enzyme and a ligase (coupled or sequentially) on a surface or in a microfluidics device.

The present disclosure provides embodiments in which purification of nucleic acids is facilitated by combining enzymatic steps into a single reaction and/or immobilizing enzymes on magnetic beads or other supports. The present disclosure further provides embodiments in which enzyme activity and/or thermostability is enhanced by immobilization on magnetic beads or other supports.

In some embodiments, a method of preparing a library (e.g., a DNA library, an RNA library) for sequencing (e.g., ONT sequencing) may include in a coupled reaction, (a) contacting a population of nucleic acid fragments with a tailing enzyme to produce tailed fragments, and/or (b) ligating to the tailed fragments a sequencing adapter with a ligase to produce adapter-tagged fragments. A method may further include separating adapter-tagged fragments from the tailing enzyme and the ligase to produce separated adapter-tagged fragments and optionally separated tailing enzyme and/or separated ligase. A tailing enzyme, in some embodiments, may be or comprise immobilized tailing enzyme. A ligase, in some embodiments, may be or comprise immobilized ligase. For example, a tailing enzyme may be immobilized on a bead (e.g., a magnetic bead) and/or a ligase may be immobilized on a bead (e.g., a magnetic bead). Each immobilized enzyme may be attached to a separate support or may be combined on a common support. Optionally, a tailing enzyme and a ligase each may be immobilized on their own separate support or both may be co-immobilized on a single support. In some embodiments, one or more enzymes (e.g., a tailing enzyme and/or a ligase) may be soluble enzymes. For example, a method may include contacting one or more soluble enzymes with one or more substrates in a liquid (e.g., aqueous) media. In some embodiments, a method may include contacting two enzymes (e.g., a tailing enzyme and a ligase) with at least one substrate for at least one of the two enzymes (e.g., DNA or RNA) in a coupled reaction. In some embodiments of a coupled reaction, at least one product of one of the enzymes is a substrate of the other enzyme. It may be desirable to select reaction conditions to favor production of the product(s) that are substrates of the other enzyme and minimize or avoid production of anything that reduces the efficiency of any of the coupled reaction enzymes, but conditions may be adjusted to tolerate the presence of some unwanted products.

In some embodiments, separating adapter tagged fragments (e.g., where one or more enzymes used are immobilized on magnetic beads) may further comprise subjecting the coupled reaction to a magnetic field. Subjecting a coupled reaction to a magnetic field may include accomplished in any manner desired. For example, a coupled reaction may be moved into an existing magnetic field, an existing magnet may be moved into effective range of a coupled reaction, or a magnetic field may be applied, for example, by switching on an electromagnet within an effective distance of a coupled reaction. In some embodiments, subjecting a coupled reaction to a magnetic field gathers magnetic beads in the coupled reaction forming a liquid fraction comprising, for example, reaction products, buffers, and solvent, but few, if any, magnetic beads) and a bead fraction comprising, for example, magnetic beads, enzymes, and solvent, but few, if any, reaction products. Gathered magnetic beads may form a pellet or other aggregate that facilitates separation (e.g., removal) of other reaction components (e.g., components remaining in solution).

A population of nucleic acid fragments may comprise ribonucleic acid fragments and/or may comprise deoxyribonucleic acid fragments. Fragments may be of any desired size. For example, a population of nucleic acid fragments may comprise fragments ranging in length from 100 to 1000 nts, 200 to 2000 nts, 500 to 5000 nts, 1,000 to 10,000 nts, 2,000 to 20,000 nts, 5,000 to 50,000 nts, 10,000 to 100,000 nts, or combinations thereof. A population of nucleic acid fragments may comprise fragments from any desirable source including, for example, fragments synthesized or assembled in vitro and/or fragments of polynucleotides from microbes (e.g., yeast, bacteria, viruses, phage), fungi, plants, amphibians, reptiles, fish, mammals, birds, or any other organism.

In some embodiments, methods may be capable of producing sequencing libraries with little input RNA. For example, methods may use a population of nucleic acid fragments having less than 100 ng of nucleic acids or a population of nucleic acid fragments having less than 10 ng of nucleic acids. In some embodiments, methods including coupled reactions and/or immobilized enzymes may produce more sequencing reads per mass of input DNA or RNA when compared with corresponding methods that do not include coupled reactions and/or immobilized enzymes. For example, methods including a coupled reaction and/or an immobilized enzyme may produce 2×, 3×, 4×, 5×, 6×, 7×, 8×, 9×, 10×, 11×, 12× or more sequencing reads compared to methods including only sequential reactions with soluble enzymes and bead purification.

The present disclosure further provides methods for preparing sequencing libraries comprise any combination of tailing and/or ligating steps and further comprising reusing the tailing enzyme and/or the ligase. For example, a method may include, in a second reaction (e.g., a second coupled reaction), contacting a second population of nucleic acid fragments with the separated tailing enzyme (produced from the first reaction) to produce additional tailed fragments, and ligating (optionally, with a ligase also recycled from the first reaction) to the additional tailed fragments a second sequencing adapter with the separated ligase to produce additional adapter-tagged fragments. The additional adapter-tagged fragments may be separated from the separated tailing enzyme and the separated ligase to produce separated additional adapter-tagged fragments, separated tailing enzyme, and/or separated ligase.

In some embodiments, a method contacting separated adapter-tagged fragments with one or more transmembrane pores (e.g., ONT nanopores) for sequencing. For example, a method may comprise translocating separated adapter-tagged fragments through one or more transmembrane pores, (f) detecting electrical changes as the one or more separated adapter-tagged fragments are translocated through the one or more transmembrane pores in an insulating membrane to produce an electrical signal; and/or analyzing the electrical signal to generate a sequence read. In some embodiments, one or more transmembrane pores (e.g., in contact with a population of adapter-tagged fragments) may retain about 90% of their initial activity (e.g., translocation activity) after two hours and/or may retain about 50% of their initial activity after 8 hours. One or more transmembrane pores, according to some embodiments of the disclosure, may produce at least 900 sequence reads per transmembrane pore. For example, the number of sequencing reads of a population of nanopores (e.g., in contact with a population of adapter-tagged fragments) may be, on average, at least 900. In some embodiments, a sequencing adapter may be a single-stranded adapter and may comprise a leader sequence; and a first sequence and a second sequence, wherein the first and second sequences are complementary to each other and define a hairpin, wherein the leader sequence is configured to thread into the one or more transmembrane pores.

In some embodiments, a wide range of enzymes may be immobilized without loss or without substantial loss of activity including, for example, Taq DNA polymerase, T4 DNA polymerase (T4 DNA pol), T4 polynucleotide kinase (T4 PNK), T4 DNA ligase, polyA polymerase, Klenow (Exo-), T4 BGT, 9° N DNA ligase, Taq DNA ligase, Bst DNA pol 2.0, phi29 DNA pol, Vvn (nuclease), Gka Reverse transcriptase, Tbr Reverse transcriptase, RNase A (catalytic mutants), PolyA polymerase, Beta-galactosidase, PNGaseF, Endo H, Endo S, Sialidase, Human carbonyl reductase, Human Aldose reductase, Drosophila aldehyde-ketone reductase (AKR), type IIS restriction enzymes, Cap-0 enzymes (e.g., Faustovirus capping enzymes, vaccinia virus capping enzyme), and Cap-1 enzymes (e.g., 2′-O-methyltransferase). In some embodiments, BspQI, a thermostable Type IIs restriction endonuclease, may be immobilized. BspQI has the recognition sequence 5′ GCTCTTC N1/N4 3′ and contains two potential catalytic sites in a single polypeptide chain produced by the BspQI-coding gene. Therefore, the sequence and structural properties of BspQI suggest that each BspQI enzyme molecule can independently recognize, bind and cleave DNA. Soluble type IIs restriction endonucleases have been used to produce a DNA template for the purpose of in vitro synthesis of RNAs possessing 3′ poly(A) tail (Mol Ther Nucleic Acids. 2016 April; 5(4): e306.). Immobilized BspQI may have a variety of unique features and advantages as described herein. In some embodiments, a unit of BspQI is the amount of enzyme required to digest 1 μg of λ DNA in 1 hour at 50° C. in a total reaction volume of 50 μl.

Each immobilized (e.g., via covalent linkage to a support) BspQI, RNA polymerase, and/or capping enzyme molecule may contain all the required catalytic elements and thus may produce the same reaction products as its soluble counterpart. Immobilization, in some embodiments, permits and/or facilitates removal of the enzyme without heat inactivation which may cause damage of the products or affect recovery of the products of the enzymatic processing. Reaction products included in a reaction mixture may be separated from enzyme molecules immobilized to solid support. Separated BspQI optionally may be reused. Reuse may optionally include reconditioning the enzyme, for example, by contacting it with fresh reaction buffer. Use of immobilized enzymes can potentially save time and cost required for removal of soluble enzymes thereby lowering the overall cost of a workflow.

Immobilization of BspQI may provide solutions for protein aggregation because upon immobilization all the BspQI protein molecules are presumably spatially separately on the surface of a solid support to avoid protein-protein interactions that may cause aggregation leading to loss of activity. According to some embodiments, an immobilized enzyme may comprise a BspQI molecule (e.g., one or more BspQI molecules, each having at least one catalytically active site) attached to a support (e.g., a bead).

The present disclosure relates, in some embodiments, to methods of cleaving a polynucleotide. For example, a method may include contacting an immobilized enzyme with a polynucleotide substrate to form reaction products, separating the immobilized enzyme from the reaction products, and optionally reusing the immobilized enzymes in one or more subsequent reactions. Reusing the immobilized enzyme may comprise, for example, repeating the contacting step with additional substrate (e.g., the same as or different from the polynucleotide substrate) to form additional products (e.g., the same as or different from the reaction products). Reusing the immobilized enzyme may further comprise separating the immobilized enzyme from the additional reaction products and, optionally, combining the reaction products with the additional products to form pooled products. Contacting, in some embodiments, may comprise cleaving one or both strands of a double stranded polynucleotide substrate. For example, a method may comprise contacting a double stranded polynucleotide with an immobilized type IIS restriction endonuclease (e.g., BspQI) to produce double stranded polynucleotide products comprising one or more nicks (e.g., breaks in the phosphate backbone). Methods may comprise contacting an immobilized type IIS restriction endonuclease with a double stranded polynucleotide substrate to form nicked polynucleotide products, contacting the nicked polynucleotide products with a second enzyme (e.g., a second immobilized enzyme) to form further polynucleotide reaction products. In some embodiments, a second enzyme may be the same or different as the enzyme included in the immobilized enzyme of the first contacting step. For example, a second enzyme may comprise a type III restriction endonuclease and/or a polymerase (e.g., a DNA polymerase or an RNA polymerase). In some embodiments, further polynucleotide reaction products may comprise at least one single-stranded polynucleotide product, for example, as a result of a second cleavage event on the original substrate or as a result of (templated or untemplated) synthesis of a nascent strand). A method may comprise separating the second enzyme (e.g., the second immobilized enzyme) from the further polynucleotide reaction products.

The present disclosure relates, in some embodiments, methods of producing RNA (e.g., mRNA) using immobilized enzymes. For example, methods may comprise contacting a polynucleotide template with an immobilized enzyme to form a cleaved (e.g., nicked) polynucleotide template and contacting the cleaved (e.g., nicked) polynucleotide template with a polymerase (e.g., an RNA polymerase) to produce the RNA (e.g., to produce transcription products comprising the RNA). Contacting the cleaved (e.g., nicked) polynucleotide template with the polymerase may further comprise contacting the cleaved (e.g., nicked) polynucleotide template with nucleoside triphosphates. In some embodiments, the polynucleotide template comprises, in a 5′-3′ direction, a coding sequence, a poly(U) sequence, and a type IIS restriction endonuclease recognition site (e.g., a BspQI recognition site). A polynucleotide template may comprise a DNA plasmid, for example, a linear or circular plasmid. An immobilized enzyme may comprise a type IIS restriction endonuclease (e.g., BspQI, Nt.BspQI), a support (e.g., a bead such as a magnetic bead), and a linker disposed between the type IIS restriction endonuclease and the support. A polymerase (e.g., an RNA polymerase) may be soluble or may be immobilized on the same support as the type IIS polymerase or a different support from the type IIS polymerase. A method of forming RNA may be performed as a coupled reaction (e.g., in a single compartment, tube, vessel or other space). In some embodiments, a method may further comprise capping the RNA produced, for example, co-transcriptionally or post-transcriptionally. Capping may comprise contacting the RNA product (e.g., mRNA) with a soluble capping enzyme.

More efficient capping of the RNA substrate may support capping with less enzyme added to a capping reaction, producing more capped RNA (as a percentage of the RNA in the reaction) using the same amount of enzyme, terminating the reaction earlier, and/or capping RNAs that have secondary structure at the 5′ end more efficiently.

Disclosed reaction conditions may be varied, including, without limitation, reaction temperature, reaction duration, reaction component concentrations (e.g., SAM, inorganic pyrophosphatase, NTPs, transcript template), enzymes (e.g., capping enzymes, polymerases, and fusions thereof), and enzyme forms (e.g., soluble, immobilized, or co-immobilized). For example, an FCE:T7 RNA polymerase fusion protein may further improve the efficiency of Cap-0 RNA synthesis in terms of fraction of Cap-0 transcripts and transcription output. In addition, inclusion of a cap 2′O methyltransferase, such as Vaccinia cap 2′O methyltransferase, in the reactions can generate Cap-1 transcript at high efficiency. Depending on which enzyme is used and the other components in the reaction mix, disclosed methods may be used to make RNAs that have a Gppp cap, a 7-methylguanylate cap (i.e., a m7Gppp cap, or “cap-0”), or an RNA that has an m7Gppp cap that has addition modifications in the first and/or second nucleotides in the RNA (i.e., “cap-1” and “cap-2”; see Fechter J. Gen. Vir. 2005 86: 1239-49). For example, if there is no SAM in the reaction mix then RNAs that have a Gppp cap may be produced. If the reaction mix comprises SAM in addition to the capping enzyme, then cap-0 RNA may be produced. If the reaction mix comprises other enzymes, e.g., cap 2′OMTase in addition to SAM, then cap-1 and/or cap-2 RNA may be produced. A reaction mix may comprise other components in addition to those explicitly described above.

In some embodiments, uncapped RNA in the reaction mix may be prepared by solid-phase oligonucleotide synthesis chemistry (see, e.g., Li, et al J. Org. Chem. 2012 77: 9889-9892), in which case the RNA in the sample may be may have a length in the range of 10-500 bases, e.g., 20-200 bases). In other embodiments, uncapped RNA in the reaction mix may be prepared in a cell free in vitro transcription (IVT) reaction in which a double-stranded DNA template that contains a promoter for an RNA polymerase (e.g., a T7, T3 or SP6 promoter) upstream of the region that is transcribed is copied by a DNA-directed RNA polymerase (typically a bacteriophage polymerase; soluble, immobilized, or co-immobilized) to produce a product that contains RNA molecules copied from the template. In either embodiment, the RNA sample that is capped in the present method contains a single species of RNA (either the synthetic oligonucleotide or the transcript). In addition, the reaction mix may be RNase-free and may optionally comprise one or more RNase inhibitors. The RNA in the sample may contain a non-natural sequence of nucleotides and in some embodiments, may contain non-naturally occurring nucleotides. In some embodiments, the in vitro transcription reaction may employ a thermostable variant of the T7, T3 and SP6 RNA polymerase (see, e.g., PCT/US2017/013179 and U.S. application Ser. No. 15/594,090). In these embodiments, the RNAs may be transcribed at a temperature of greater than 44° C. (e.g., a temperature of at least 45° C., at least 50° C., at least 55° C. or at least 60° C., up to about 70° C. or 75° C.) in order to reduce the immunogenicity of the RNA (see, e.g., WO 2018/236617). In some cases, the uncapped RNA may be capped immediately after it is made, e.g., by adding an RNA capping enzyme and GTP/modified GTP, as needed to the in vitro transcription reaction after the reaction has run its course. In some embodiments, the RNA made in an in vitro transcription reaction may purified prior to capping.

In some embodiments, the RNA in the sample may be a therapeutic RNA. In these embodiments, the product of the present method may be used without purification of the capped RNA from the uncapped RNA. In these embodiments, the product of the present reaction may be combined with a pharmaceutical acceptable excipient to produce a formulation, where “pharmaceutical acceptable excipient” is any solvent that is compatible with administration to a living mammalian organism via transdermal, oral, intravenous, or other administration means used in the art. Examples of pharmaceutical acceptable excipients include those described for example in US 2017/0119740. The formulation may be administered in vivo, for example, to a subject, examples of which include a human or any non-human animal (e.g., mouse, rat, rabbit, dog, cat, cattle, swine, sheep, horse or primate). Depending on the subject, the RNA (modified or unmodified) can be introduced into the cell directly by injecting the RNA or indirectly via the surrounding medium. Administration can be performed by standardized methods. The RNA can either be naked or formulated in a suitable form for administration to a subject, e.g., a human. Formulations can include liquid formulations (solutions, suspensions, dispersions), topical formulations (gels, ointments, drops, creams), liposomal formulations (such as those described in: U.S. Pat. No. 9,629,804 B2; US 2012/0251618 A1; WO 2014/152211; US 2016/0038432 A1). The cells into which the RNA product is introduced may be in vitro (i.e., cells that cultured in vitro on a synthetic medium). Accordingly, the RNA product may be transfected into the cells. The cells into which the RNA product is introduced may be in vivo (cells that are part of a mammal). Accordingly, the introducing may be done by administering the RNA product to a subject in vivo. The cells into which the RNA product is introduced may be present ex vivo (cells that are part of a tissue, e.g., a soft tissue that has been removed from a mammal or isolated from the blood of a mammal).

Synthesis of large amounts of uniformly capped mRNA transcripts in a cost-effective and streamlined manner that is scalable to support multi-gram synthesis may be desired or required to manufacture mRNA for therapeutic applications. Current approaches to mRNA manufacturing use costly mRNA cap analogs in in vitro transcription reactions. Alternatively, separate reactions are required to produce 5′-triphosphate RNA by in vitro transcription followed by mRNA capping using enzymes—a more complex process that is harder to scale. A single-step in vitro synthesis of capped RNA using T7 RNA polymerase and a capping enzyme (e.g., each in soluble, immobilized, or co-immobilized forms) may streamline the manufacturing process. Single-vessel reactions with both enzymes may reduce or remove otherwise prohibitive costs of synthetic mRNA cap analogs and may reduce the complexity of scaling this workflow to support multi-gram (and beyond) synthesis.

A capping method, in some embodiments, may comprise contacting an RNA polymerase (e.g., a T7 RNA polymerase, a thermoactive Hi-T7 RNA polymerase and/or an FCE fusion), a polynucleotide (e.g., DNA or RNA) encoding a target RNA, a capping enzyme (e.g., VCE, FCE, an FCE fusion), NTPs, a buffering agent, optionally in the presence or absence of SAM, optionally wherein the RNA polymerase, the capping enzyme, or both the RNA polymerase and the capping enzyme are immobilized enzymes (e.g., immobilized on a magnetic bead or agarose support). Nucleoside triphosphates (NTPs) may include unmodified ATP, modified ATP (m6ATP, m1ATP), unmodified CTP, modified CTP (e.g. m5CTP), unmodified GTP, modified GTP, unmodified UTP, modified UTP (e.g., pseudouridine triphosphate, m1pseudouridin triphosphate), NTPs containing 2′O methylation, and/or combinations thereof. In some embodiments, a capping method may comprise contacting a capping enzyme (e.g., VCE, FCE, an FCE fusion), a target RNA, and guanosine triphosphate (GTP), and/or modified GTP. A capping method may comprise, in some embodiments, contacting a capping enzyme (e.g., VCE, FCE, an FCE fusion), a target RNA, and guanosine triphosphate (GTP) and/or modified GTP, an O-methyl transferase (e.g., thermoactive Vaccinia mRNA cap 2′O methyltransferase), in the presence or absence of SAM.

Contacting, according to some embodiments, may be performed in a single location (e.g., in a single step), for example, on any surface (e.g., plate or bead) or in any vessel (e.g., tube, flask, vial, column, container, bioreactor or other space). In some embodiments, contacting may comprise contacting some or all the subject elements in any desired order or concurrently. Contacting, in some embodiments, may comprise contacting some or all the subject elements in the presence of a buffering agent. For example, contacting may include contacting, in any order or concurrently, a capping enzyme (e.g., VCE, FCE, an FCE fusion), a target RNA, GTP, and a buffering agent in the presence or absence of SAM. In some embodiments, contacting may further comprise contacting at a temperature of 40° C. to 60° C. (e.g., at 40° C., 42° C., 44° C., 45° C., 46° C., 48° C., 50° C., 52° C., 54° C., 55° C., 56° C., 58° C., or 60° C.) for any desired period of time, for example, 1 minute to 120 minutes (e.g., 1 minute, 5 minutes, 10 minutes, 15 minutes, 20 minutes, 25 minutes, 30 minutes, 45 minutes, 60 minutes, 75 minutes, 90 minutes, 105 minutes, or 120 minutes). In any of the embodiments disclosed herein, contacting may further comprise contacting at a first temperature of 25° C. to 60° C. and cooling or warming to a second temperature of 25° C. to 60° C., different from the first temperature, wherein, optionally, at least one of the first temperature or second temperature is not less than 40° C. A first temperature may be selected to produce, stabilize and/or retain a desired amount of uncapped target RNA and a second temperature may be selected to produce, stabilize and/or retain a desired amount capped target RNA. Each temperature may be maintained for any desired period of time (e.g., 1 minute to 120 minutes). For example, a first temperature may be 37° C. for a first period of 1 to 30 minutes and a second temperature may be 28° C., 32° C., 37° C., 45° C., or 50° C. for a second period of 1 to 30 minutes. Contacting, in any of the disclosed embodiments, may include placing one item in direct contact with another, placing two items together on a surface or in a vessel in sufficient proximity that they may physically and/or chemically interact with or without further agitation or mixing. Contacting may include the initial act of placing one item in direct contact with or proximity to another and/or maintaining conditions that permit or favor the two items to contact each other.

A method, in some embodiments, may comprise synthesizing RNA (e.g., directed or undirected by a template) in vitro to produce an uncapped target RNA and capping the target RNA (e.g., in single-step reactions). Synthesizing RNA from a template may comprise, for example, contacting an RNA polymerase (e.g., a T7 RNA polymerase, a thermoactive Hi-T7 RNA polymerase and/or an FCE fusion) with the template (e.g., an RNA template or a DNA template) and one or more NTPs to produce an uncapped target RNA, a buffering agent, and optionally in the presence or absence of SAM. Nucleoside triphosphates (NTPs) may include unmodified ATP, modified ATP (m6ATP, ml ATP), unmodified CTP, modified CTP (e.g. m5CTP), unmodified GTP, modified GTP, unmodified UTP, modified UTP (e.g., pseudouridine triphosphate, m1pseudouridine triphosphate), NTPs containing 2′O methylation, and/or combinations thereof. Capping a target RNA may comprise, for example, contacting FCE (e.g., FCE or an FCE fusion) with a target RNA and guanosine triphosphate (GTP) and/or a modified GTP to produce a capped target RNA. In some embodiments, a capping method may comprise contacting a target RNA with an FCE fusion at a temperature of 25° C. to 60° C., A capping method may comprise, in some embodiments, contacting a target RNA with a decapping enzyme to remove an existing cap and/or assure that it is uncapped.

Capping may comprise, for example, contacting a capping enzyme (e.g., VCE, FCE, an FCE fusion, in each case, soluble, immobilized, or co-immobilized) with a target RNA, an O-methyltransferase (e.g., thermoactive Vaccinia mRNA cap 2′O methyltransferase), SAM, guanosine triphosphate (GTP), modified GTP, and/or a buffering agent. For example, capping may comprise contacting a capping enzyme, a target RNA, GTP (optionally, with or without modified GTP), and optionally a buffering agent. Capping may comprise contacting, in a single vessel (e.g., in a single step), a capping enzyme, a target RNA, GTP (optionally, with or without modified GTP), an O-methyltransferase, SAM, and optionally a buffering agent. Capping may comprise capping a pre-existing target RNA and/or synthesizing and capping a target RNA in a single vessel (e.g., in a single step), for example, by contacting a target RNA template (DNA or RNA) encoding the target RNA, a polymerase (e.g., a T7 RNA polymerase, a thermoactive Hi-T7 RNA polymerase and/or an FCE fusion), NTPs (optionally including or excluding one or more modified NTPs), a capping enzyme (e.g., VCE, FCE, an FCE fusion), and/or a buffering agent.

According to some embodiments,

Compositions

The present disclosure provides a variety of compositions for template preparation, RNA transcription, and RNA capping in one or more coupled reactions and/or using one or more immobilized enzymes. In some embodiments, a composition (e.g., a reaction mix) may comprise a target RNA (e.g., in an RNA sample) and/or a DNA template encoding a target RNA. A composition, according to some embodiments, may comprise an RNA polymerase (e.g., T7 RNA polymerase) optionally soluble or immobilized on a support and optionally may further comprise one or more NTPs. In some embodiments, a composition may comprise a capping enzyme (e.g., VCE, FCE, 2′OMT) optionally soluble or immobilized on a support and optionally may further comprise one or more of GTP, SAM and a buffering agent. A composition may comprise, in some embodiments, two or more enzymes. For example, a composition may comprise a first enzyme immobilized on a first support and a second enzyme immobilized on a second support. A composition may comprise a first enzyme and a second enzyme wherein the first and second enzymes are co-immobilized on a common support. The first and second enzyme may be co-immobilized to the common support as separate enzymes or as a fusion protein. A composition may have a temperature in the range of 37° C.-60° C., e.g., 37° C.-42° C., 42° C.-47° C., 47° C.-52° C. or 52° C.-60° C. In some embodiments, a composition may be free of RNase activity as a result of RNases being inhibited, inactivated or absent. For example, an RNase-free composition may comprise one or more RNase inhibitors. In some embodiments, a composition may further comprise a DNA template operably linked to a promoter, a bacteriophage polymerase that initiates transcription at the promoter, and NTPs, for transcribing the RNA. In some embodiments, a composition may include a cap 2′OMTase. A composition, according to some embodiments, may comprise an FCE fusion comprising, in N-terminal to C-terminal order, (a) Faustovirus RNA capping enzyme (e.g., an FCE RNA capping enzyme) and an RNA polymerase or (b) an RNA polymerase and an FCE RNA capping enzyme, wherein the FCE fusion optionally may further comprise a leader (e.g., operably positioned within the fusion) and/or a linker positioned between the FCE and the RNA polymerase. In some embodiments, any or all of the components referenced in this paragraph may be combined or otherwise included in a single container. Components, for example, components in a single container, may be present individually or collectively in dry (e.g., anhydrous and/or lyophilized) form. A container with one or more of the referenced components may also contain an aqueous medium.

### Kits

The present disclosure further relates to kits including immobilized enzymes. For example, a kit may include an immobilized tailing enzyme, an immobilized ligase, a polynucleotide (e.g., a population of polynucleotides) dNTPs, rNTPs, primers, buffering agents, and/or combinations thereof. In some embodiments, a kit may comprise an immobilized type IIS restriction endonuclease and one or more of a second enzyme (e.g., an additional nuclease and/or a polymerase), a capping enzyme, dNTPs, rNTPs, primers, buffering agents, and/or combinations thereof. Immobilized enzymes may be included in a storage buffer (e.g., comprising glycerol and a buffering agent). A kit may include a reaction buffer which may be in concentrated form, and the buffer may contain additives (e.g. glycerol), salt (e.g. KCl), reducing agent, EDTA or detergents, among others. A kit comprising dNTPs may include one, two, three of all four of dATP, dTTP, dGTP and dCTP. A kit comprising rNTPs may include one, two, three of all four of rATP, rUTP, rGTP and rCTP. A kit may further comprise one or more modified nucleotides. A kit may optionally comprise one or more primers (random primers, bump primers, exonuclease-resistant primers, chemically-modified primers, custom sequence primers, or combinations thereof). One or more components of a kit may be included in one container for a single step reaction, or one or more components may be contained in one container, but separated from other components for sequential use or parallel use. The contents of a kit may be formulated for use in a desired method or process.

A kit is provided that contains: (i) an immobilized tailing enzyme; and (ii) a buffer or (i) an immobilized tailing enzyme; (ii) an immobilized ligase, and (iii) a buffer. An immobilized enzyme may have a lyophilized form or may be included in a buffer (e.g., an aqueous buffer, a storage buffer or a reaction buffer in concentrated form). A kit may contain the immobilized enzyme in a mastermix suitable for receiving and amplifying a template nucleic acid. An immobilized enzyme may be a purified enzyme so as to contain substantially no DNA or RNA and/or no nucleases. A reaction buffer for and/or storage buffers containing an immobilized enzyme may include non-ionic, ionic e.g. anionic or zwitterionic surfactants and crowding agents. A kit may include an immobilized enzyme and a reaction buffer in a single tube or in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or sub-packaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### EXAMPLES

Some embodiments may be illustrated by one or more of the examples provided herein.

### Example 1: Immobilization of Poly(A) Polymerase and Kinetics Study

### Example 1A. Soluble Poly(A) Polymerase Alone

Poly(A) polymerase catalyzes poly(A) tailing at the 3′ end of RNA and the resulting tails can be hybridized with and ligated to adapters (e.g., Nanopore Adaptors) for direct RNA sequencing. The kinetics of poly(A) polymerase (NEB M0276) at different concentrations was studied as described in this Example 1. Reaction components (6 μL nuclease-free water, 1 μL 10× poly(A) polymerase reaction buffer (NEB), 1 μL 10 mM ATP, 0.5 μL RNase inhibitor, 1 μL 1 μM RNA 45-mer oligo and 0.5 μL poly(A) polymerase (at 12 nM, 24 nM, 60 nM or 120 nM)) were mixed and incubated at 37° C. for 20 min to allow poly(A) tailing. Each reaction was quenched by addition of 10 μL 50 mM EDTA with 0.7% Tween-20, diluted to a final volume of 200 μL, and sent for capillary electrophoresis (CE) analysis.

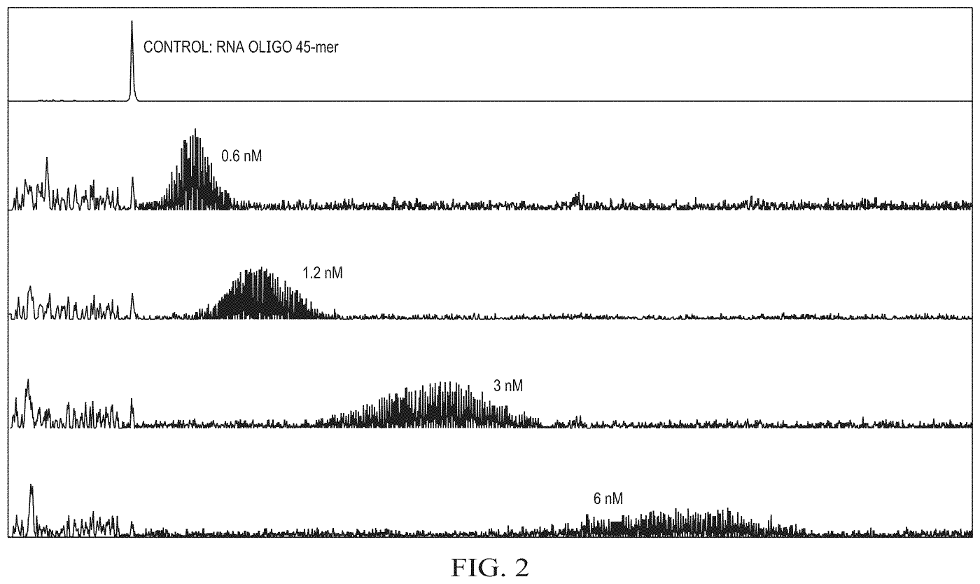

Results shown in FIG. 2 demonstrate that RNA oligo strands were extended by addition of poly(A) tails at the 3′ end of the RNA with the presence of poly(A) polymerase. More extensive strand extension was observed with increasing the concentration of poly(A) polymerase from 0.6 nM to 6 nM final concentration.

### Example 1B. Poly(A) Polymerase—SNAP-Tag® Fusion

Cells expressing a poly(A) polymerase—SNAP-Tag® fusion were harvested by centrifugation and lysed by sonication on ice. The resulting lysate was centrifuged and the clarified crude extract produced was purified on a nickel column. After loading, the column was washed and the fusion protein was eluted and dialyzed overnight. The enzyme concentration was determined using Bradford assay.

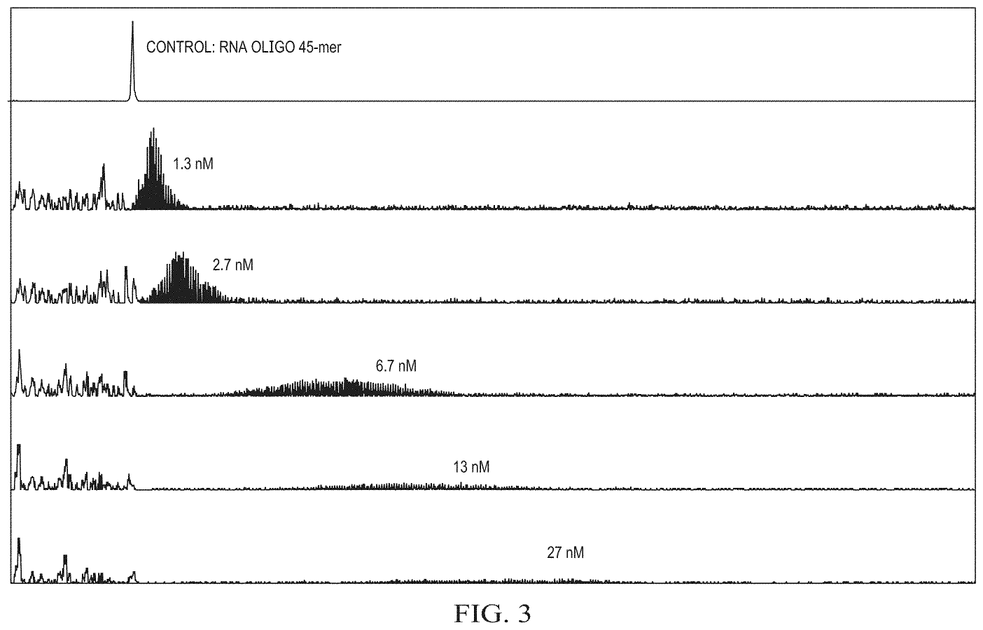

The activity of the expressed fusion protein was evaluated according to Example 1A. Results shown in FIG. 3 demonstrate RNA 45-mer oligo strand extension by the purified poly(A) polymerase—SNAP fusion protein. Comparing FIG. 2 and FIG. 3 demonstrates that RNA 45-mer oligo strand extension by the poly(A) polymerase—SNAP fusion protein aligned well with the soluble NEB poly(A) polymerase.

### Example 1C. Poly(A) Polymerase Immobilization on O⁶-Benzylguainine (BG) Magnetic Beads

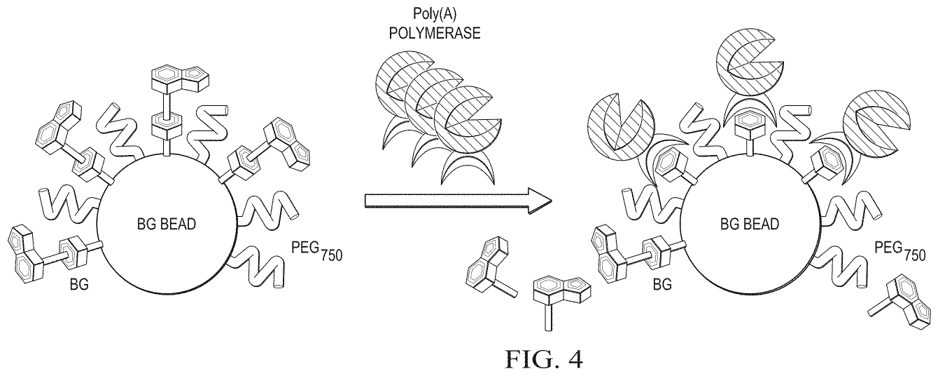

O⁶-benzylguainine (BG) functionalized magnetic beads coated with PEG₇₅₀ (100 μL of a 25% (v/v) slurry) were washed five times with 250 μL buffer (1×PBS, #9808, Cell Signaling, 1 mM DTT, 300 mM NaCl) for 5 times. Poly(A) polymerase—SNAP fusion protein (25 μg) in 125 μL buffer (1×PBS with 300 mM NaCl), was mixed with the pre-washed BG beads, and incubated at 4° C. overnight to immobilize the fusion protein (FIG. 4). The enzyme bead mixture was washed with the same buffer 8 times to remove unbound protein. Diluent C buffer without BSA (NEB) was used to resuspend the beads with immobilized fusion protein for storage at −20° C.

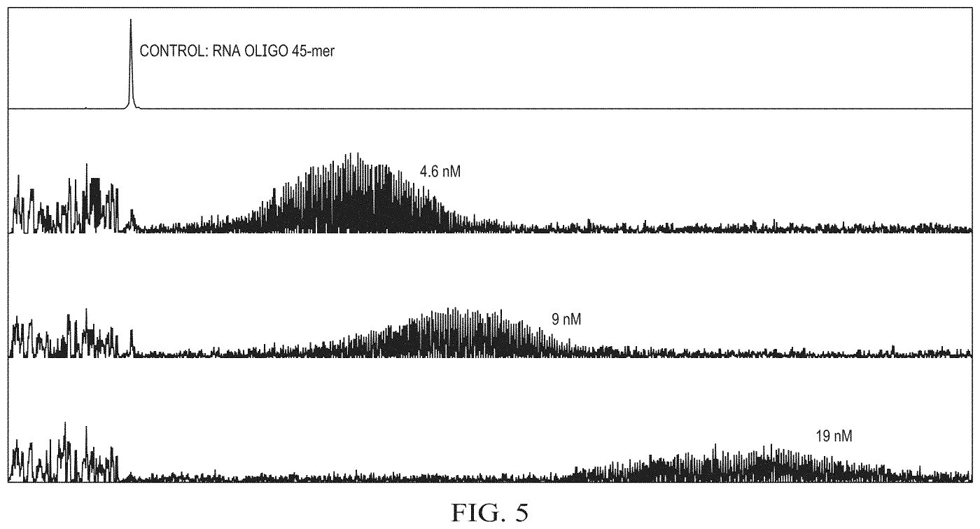

The activity of the immobilized poly(A) polymerase was evaluated according to Example 1A. Results shown in FIG. 5 demonstrate RNA 45-mer oligo strand extension by the immobilized poly(A) polymerase—SNAP fusion protein.

### Example 2: Immobilized Poly(A) POLYMERASE DISPLAYS STABILITY Including Thermostability

This example shows how to improve microenvironment for immobilized enzymes by increasing hydrophilicity of bead surface by PEG coating. Poly(A) polymerase was immobilized to two types of O⁶-benzylguainine (BG) functionalized magnetic beads coated with or without PEG₇₅₀ generally as described in Li, S et al, “Enhancing Multistep DNA Processing by Solid-Phase Enzyme Catalysis on Polyethylene Glycol Coated Beads” Bioconjugate Chem. 2018, 29, 7, 2316-2324. An aliquot of 100 μL of 25% (v/v) bead slurry was washed five times with 250 μL buffer (1×PBS, #9808, Cell Signaling, 1 mM DTT, 300 mM NaCl) for 5 times. Poly(A) polymerase—SNAP fusion protein (25 μg) was dissolved in 125 μL buffer (1×PBS with 300 mM NaCl), combined with the washed BG beads, and incubated at 4° C. overnight to immobilize the fusion protein on the beads. The immobilized poly(A) polymerase—SNAP fusion protein beads were washed with the same buffer 8 times to remove any unbound protein. Diluent C buffer (NEB) with no BSA was used to resuspend the beads with immobilized fusion protein for storage at −80° C.

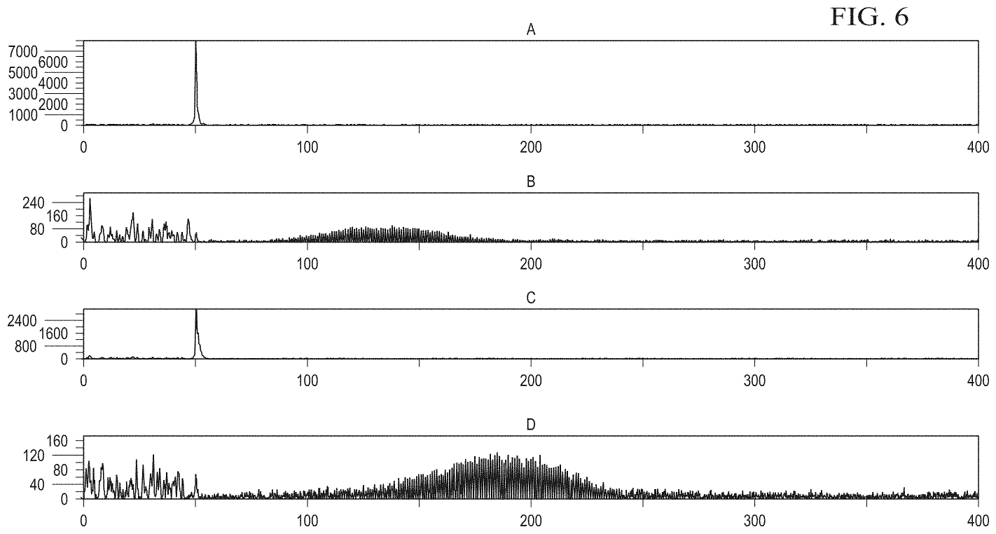

Poly(A) tailing reactions were performed using the soluble and immobilized poly(A) polymerase according to the protocols described in Example 1. The data shown in FIG. 6 demonstrate that SNAP-tagged poly(A) polymerase immobilized to PEG750-coated magnetic beads displayed poly(A) tailing activity on a 45-mer RNA oligo whereas the same fusion protein immobilized to magnetic beads without PEG₇₅₀ coating displayed little, if any, detectable poly(A) tailing.

### Example 3: Immobilized T4 DNA Ligase Displays Stability Including Thermostability

### Example 3A. T4 DNA Ligase Immobilization on Magnetic Beads and Stability Assays

This example provides immobilization of SNAP-tagged T4 DNA Ligase to BG-magnetic Beads and validation of storage stability at −20° C. and 25° C.

HS-T4 DNA Ligase protein was immobilized to BG-Magnetic-Beads by mixing 100 μg protein per 400 μl of 25% (V/V) bead slurry at 4° C. overnight in 1×PBS buffer containing 1 mM DTT, followed by extensive wash (8×). The resulting immobilized enzyme was termed BG-HS-T4 DNA Ligase and stored at −20° C. or 25° C. for 7 days. Activity testing was performed according to the Determination of the Unit Activity of T4 DNA Ligase by Capillary Electrophoresis (CE) activity assay (One unit is defined as the amount of enzyme required to give 40% to 70% (55%±15%) ligation of 0.12 μM of synthesized double-stranded DNA oligos with Hind III ends in 20 minutes at 16° C.

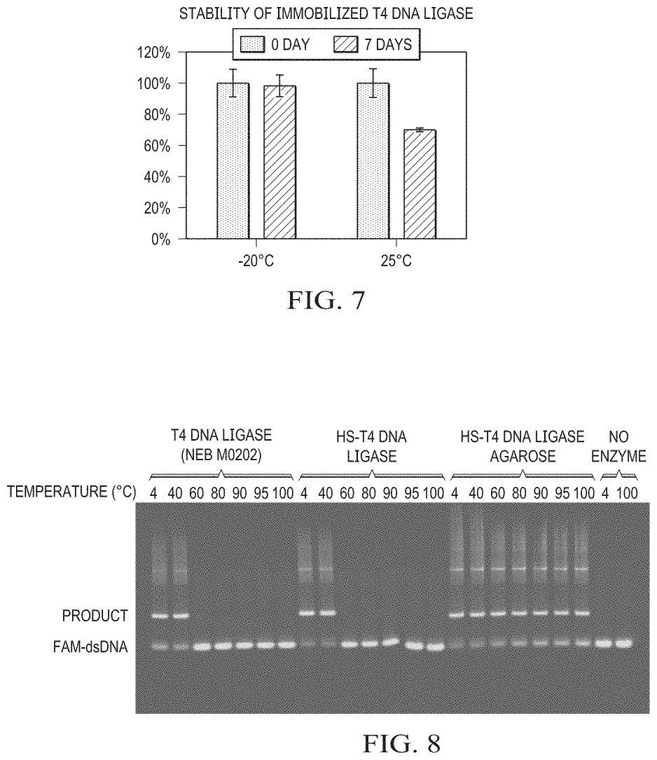

Results are shown in FIG. 7. No detectable decrease in enzyme activity was observed at −20° C. and an approximately 30% reduction in ligase activity at 25° C. during the storage period.

### Example 3B. T4 DNA Ligase Immobilization on Agarose Beads

This example demonstrates that immobilization can improve thermostability of SNAP-tagged T4 DNA ligase conjugated to BG-Agarose beads (HS-T4 DNA Ligase Agarose) compared to free SNAP-tagged T4 DNA ligase (HS-T4 DNA Ligase) or untagged T4 DNA ligase (NEB M0202). HS-T4 DNA Ligase protein was immobilized to SNAP-Capture Pull-Down Resin (a highly crosslinked agarose, NEB S9144), termed BG-Agarose, by mixing 100 μg protein per 100 μl of 50% bead slurry at 4° C. overnight in 1×PBS buffer containing 1 mM DTT, followed by extensive wash. The resulting immobilized enzyme was termed HS-T4 DNA Ligase Agarose. Each immobilized enzyme master mixture was made by mixing 32 μL of HS-T4 DNA Ligase Agarose, 20 μL of 10×T4 DNA Ligase Reaction Buffer and 74.64 μL of H₂O; Two types of soluble enzyme master mixtures were made by mixing 8 μL of T4 DNA Ligase (NEB M0202) or HS-T4 DNA Ligase, 20 μL of 10×T4 DNA Ligase Reaction Buffer and 98.64 μL of H₂O.

### Example 3C. Comparison of Ligase Activity of Soluble and Immobilized Ligases

A FAM-labeled DNA duplex was formed by annealing synthetic oligomer, Gene32FAM-fw3′A, /56-FAMN/CA TGG TGA TTA CGA TTC TTG CCC AGT ATG TCA ATA CAT CAG TAA AAA TA (SEQ ID NO:1) and Gene32-rv5′p, /5Phos/AT TTT TAC TGA TGT ATT GAC ATA CTG GGC AAG AAT CGT AAT CAC CATG (SEQ ID NO:2). A DNA substrate mixture was prepared by mixing 60 μL of 10 μM 5′FAM-labeled DNA duplex with 3′A and 160.08 μL of 15 μM TA-Adaptor possessing a 3′T, 5′-/5Phos/GAT CGG AAG AGC ACA CGT CTG AAC TCC AGT C/ideoxyu/A CAC TCT TTC CCT ACA CGA CGC TCT TCC GAT CT-3′ (SEQ ID NO:3).

For heat treatment, an aliquot of 15.83 μL from an enzyme master mixture was incubated at 4, 40, 60, 80, 90, 95 or 100° C. for 10 min, followed by addition of 9.17 μL of the DNA substrate mixture. All the ligation reactions were carried out at 4° C. overnight in a shaker. The samples were analyzed by electrophoresis on a 12% Tris-Glycine PAGE (Novex/Invitrogen) in 1×TAE Buffer for 2.5 hours at 25 mA (current). Results are shown in FIG. 8. The DNA species possessing FAM probe signal was detected by scanning the PAGE gel with an 488 nm excitation wavelength on Typhoon Imager (GE Healthcare). DNA ligation resulted in formation of new species of larger molecular mass, absent in the control reactions without ligase (No Enzyme). Both untagged T4 DNA ligase and soluble HS-T4 DNA Ligase showed no detectable ligase activity after treatment in the temperature range of 60-100° C., indicating that soluble form was subjected to irreversible denaturation. In contrast, HS-T4 DNA Ligase immobilized to BG-Agarose beads retained enzymatic activity after treatment in the same range of elevated temperature tested.

### Example 3D. Effect of Heat Treatment on Various Soluble and Immobilized Products of T4 DNA Ligases

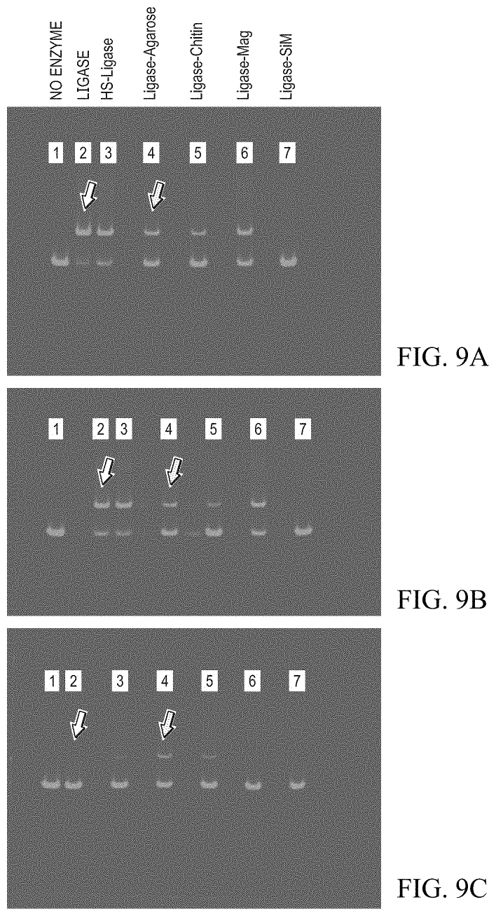

Four types of beads, Agarose (SNAP-Capture Pull-Down Resin, NEB S9144), Chitin, Magnetic beads (Mag), SiMag beads (SiM) were modified to possess benzylguanine ligand, a substrate of SNAP-tag. SNAP-tagged T4 DNA Ligase (HS-T4 DNA Ligase) protein was immobilized to each type of benzylguanine-functionalized beads. A typical immobilization reaction was performed by mixing 100 μg protein with an Agarose bead slurry at 4° C. overnight, followed by extensive wash. The resulting immobilized enzyme was termed Ligase-Agarose, Ligase-Chitin, Ligase-Mag and Ligase-SiM, respectively. Ligation reactions were set up by mixing the following components in a final volume of 20 μL containing 1×T4 DNA Ligase Reaction Buffer, 0.5 μM FAM-labeled DNA duplex, 3.75 μM adaptor, and 1 μL of immobilized HS-T4 DNA Ligase or HS-T4 DNA Ligase (HS-Ligase) or T4 DNA Ligase (Ligase, NEB M0202S). The reaction mixtures were incubated for 30 min at 4° C. (A), 37° C. (B) or 45° C. (C). Subsequently, all the reaction mixtures were incubated for 2 hours at 23° C. for DNA ligation to proceed. The samples were analyzed by electrophoresis on a 12% Tris-Glycine PAGE (Novex/Invitrogen) in 1×TAE Buffer for 2.5 hours at 25 mA (current). The DNA species possessing FAM signal was visualized by scanning the PAGE gel with an 488 nm excitation wavelength on Typhoon Imager (GE Healthcare). Results are shown in FIGS. 9A-9C. DNA ligation resulted in formation of a product of higher molecular mass, which is absent in the control reactions without ligase (No Enzyme).

All ligase-containing reactions except for Ligase-SiM showed ligase activity after treatment at 4° C. (FIG. 9A) or 37° C. (FIG. 9B) for 30 min. FIG. 9C shows that after heat treatment at 45° C. HS-T4 DNA Ligase immobilized to BG-Agarose beads retained higher enzymatic activity compared to the other immobilized ligase products. Both untagged T4 DNA ligase (Ligase) and soluble HS-T4 DNA Ligase (HS-Ligase), however, showed no or residual ligase activity after treatment at 45° C. for 30 min, indicating that these soluble form ligases was subjected to irreversible denaturation.

### Example 3E. Effect of Heat Treatment on Various Soluble and Immobilized Products of T4 DNA ligases

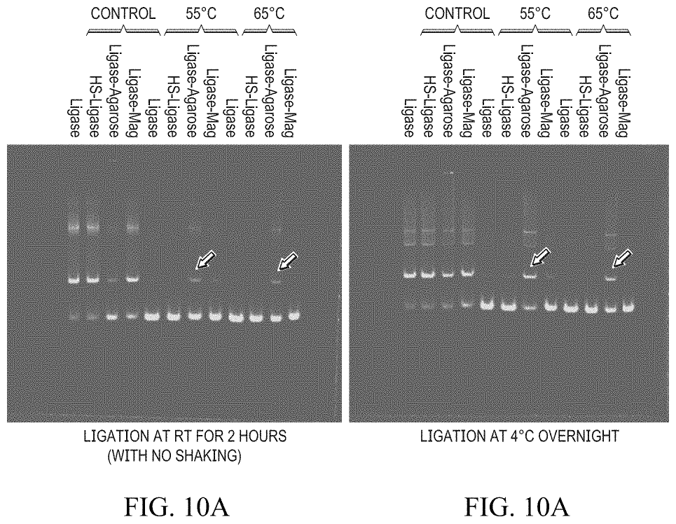

SNAP-tagged T4 DNA Ligase (HS-T4 DNA Ligase) protein was immobilized to BG-Agarose Beads (SNAP-Capture Pull-Down Resin, NEB 59144) and BG-Magnetic Beads (Mag, 1 μm) functionalized with benzylguanine ligand. The resulting immobilized enzyme was termed Ligase-Agarose and Ligase-Mag, respectively. Ligation reactions were set up by mixing the following components in a final volume of 20 μL containing 1×T4 DNA Ligase Reaction Buffer, 0.5 μM FAM-labeled DNA duplex, 3.75 μM adaptor, and 1 μL of immobilized HS-T4 DNA Ligase or HS-T4 DNA Ligase (HS-Ligase) or T4 DNA Ligase (Ligase, NEB M0202S). The reaction mixtures were incubated for 10 min at 4° C., 55° C. (B) or 65° C. Subsequently, the reaction mixtures were incubated either at 23° C. for 2 hours (FIG. 10A) or at 4° C. overnight (FIG. 10B). The samples were electrophoresed on a 12% Tris-Glycine PAGE (Novex/Invitrogen) in 1×TAE Buffer for 2 hours at 25 mA/gel. The DNA species possessing FAM probe were visualized by scanning the PAGE gel with an 488 nm excitation wavelength on Typhoon Imager (GE Healthcare). Results are shown in FIGS. 10A-10B. All positive control reactions (4° C.) displayed ligase activity. Untagged T4 DNA ligase (Ligase) and soluble HS-T4 DNA Ligase (HS-Ligase) as well as HS-T4 DNA Ligase immobilized onto Magnetic Beads (Ligase-Mag) showed no or residual ligase activity after treatment at 55° C. or 65° C. In contrast, HS-T4 DNA Ligase immobilized to BG-Agarose Beads exhibited similar enzymatic activity for each series of ligation reactions when pre-treated at 4° C., 55° C. or 65° C., indicating that immobilization to BG-Agarose Beads improved heat resistance of T4 DNA Ligase.

### Example 3F. Reusing T4 DNA Ligase to Incorporate Unique Molecular Identifiers (UMIs)

For next-generation sequencing, barcoding is an effective and commonly used approach in multiplexed deep sequencing experiments. During the demultiplexing step, identification of UMIs (barcodes) enables calling and quantification of the individual libraries which are pooled for a single sequencing run. Furthermore, UMIs are increasingly used to track nucleic acids from individual cells and to quantitatively assess their clonal contributions over time. This example provides a workflow for efficiently producing libraries with UMIs that reuses immobilized enzymes.

A typical library preparation protocol may consist of (a) repairing the ends of the members of a population of nucleic acids, (b) A/dA-tailing repaired members of the population, (c) ligating adapters to A/dA tailed members of the population, and (d) bead purification of adapter-tagged members of the population. Using immobilized enzymes in accordance with this example obviates the need for bead purification and allows enzymes to be reused in subsequent cycles of library preparation.

In each cycle, a nucleic acid library may be ligated to an adapter with a bar code using immobilized enzymes in accordance with Example 4E to produce an adapter tagged library. Immobilized enzyme beads (IM-Poly(A) polymerase and IM-ligase) are extensively washed, for example, at least 5 times to remove residual barcoded adaptor, as demonstrated in the experiment below and retained for reuse in the next cycle. A wash step can be incorporated to wash away residual bar-coded adaptor in each cycle before an adaptor possessing a different barcode is ligated to RNA species from a fresh RNA sample. The number of cycles may be varied, and all resulting adapter-tagged libraries may be pooled for multiplex sequencing.

In this example, a preparation of 300 units of T4 DNA ligase immobilized onto magnetic beads was utilized to perform repeated ligation of two adaptors used for library construction for Nanopore direct RNA sequencing. One of the adaptor sequences was labeled with a 5′ FAM probe to detect and quantify the ligation product using capillary electrophoresis. In each reaction cycle, (a) an RNA library and the adapters were added to a vial containing the immobilized ligase and incubated at 25° C. for 10 min; (b) the enzyme-bearing beads were pelleted on magnetic rack; (c) the product-containing supernatant was removed from the vial and transferred for CE analysis; and (d) the pelleted beads were washed 5 times in conjunction with micro-centrifugation in preparation for the next adaptor ligation cycle.

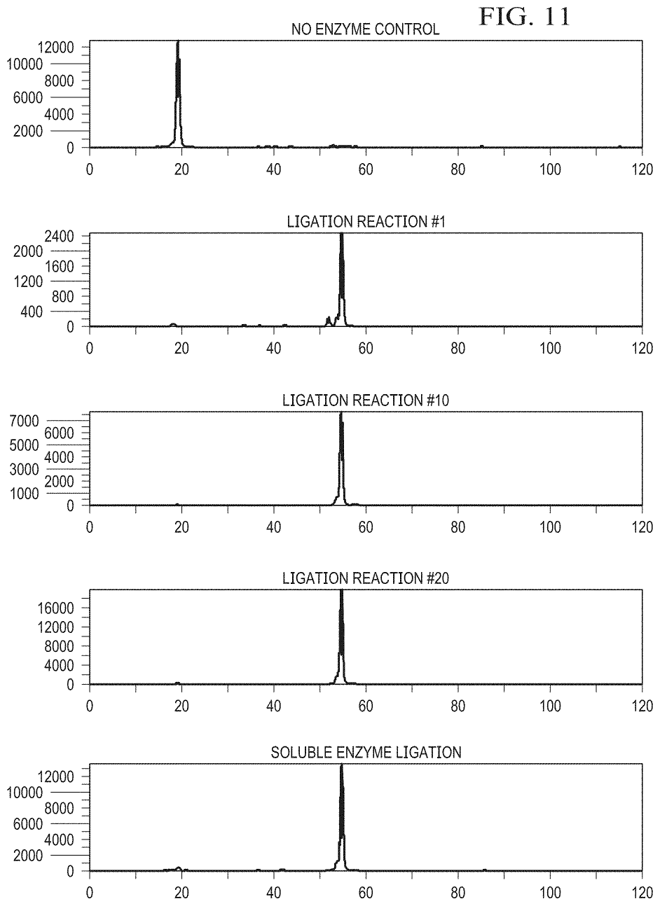

Results are shown in FIG. 11 and demonstrate efficient ligation in 20 consecutive ligation reactions, which is indicative of reliability and reproducibility of immobilized T4 DNA ligase. In the control, soluble T4 DNA ligase was used to carry out a single ligation reaction for the same adaptor substrates.

### Example 4: Library Preparation Using Soluble and Immobilized Enzymes and Nanopore Direct RNA Sequencing

Nanopore direct RNA sequencing was performed using libraries prepared according to one of the five methods described in this example and illustrated in FIGS. 1A-1E. Total RNA from Listeria monocytogenes was extracted using NEB Monarch Total RNA Miniprep Kit (NEB #T2010) and DNase I pack (NEB #T2019L) according the protocols of the manufacturer. The concentration of purified total RNA was measured using Invitrogen Qubit RNA High-sensitivity Assay Kit (cat. Number: Q32852).

Details of RNA library preparation for each approach are discussed below. In all cases in this Example 4, sequencing preparation began with 500 ng of each RNA as recommended by Oxford Nanopore Technologies. For the libraries prepared with soluble enzyme with bead purification, Nanopore's bead purification protocol was adopted. After bead purification 20 μL of the resulting RNA library was mixed with 17.5 μL of nuclease-free water and 37.5 μL of RNA running buffer (provided by ONT) to a final volume of 75 μL before loading into a flow cell for direct RNA sequencing. For the libraries prepared with soluble enzyme without bead purification, and immobilized enzymes, a portion of each 40 μL RNA library was supplemented with nuclease-free water to 37.5 μL, and mixed with 37.5 μL RNA running buffer to a final volume of 75 μL.

Direct RNA sequencing was performed on a MinION® MkIb with R9.4 flow cells. MinKNOW® instrument software (ONT) recorded the nanopore current as each strand of an adaptor-ligated RNA translocated through a nanopore. Albacore 1.2.1 (ONT) was used to perform base-calling. A report that displayed the major data sets was generated for each sequencing. Major parameters, such as direct RNA reads and average read length, were compared.

### Example 4A. Soluble Poly(A) Polymerase Without Bead Purification (FIG. 1A)

For poly(A) tailing, mix 8 μL quick ligation buffer, 1.2 μL 5 M NaCl solution, 0.5 μL poly(A) polymerase (NEB M0276), and 500 ng total Listeria monocytogenes RNA, supplemented with nuclease-free water to 30 μL in a 0.2 mL thin-walled PCR tube. Incubate the reaction at 37° C. for 20 min. Next, for adaptor ligation, add 1.0 μL RT Adaptor (RTA), 6.0 μL RNA Adaptor (RMX) and 3.0 μL T4 DNA ligase (NEB M0202M) to the poly(A) tailed RNA sample to make a final volume of 40 μL. Incubate the reaction at 25° C. for 10 min. RNA concentration was measured using the Qubit method after the enzymatic reaction (FIG. 1A). An aliquot of 40 μL RNA sample was used for further library prep as described above.

### Example 4B. Sequential Reactions with Soluble Enzymes and Bead Purification (FIG. 1B)

Library preparation according to Example 4A was repeated with the addition of a bead purification step after the enzymatic reactions. Specifically, 40 μL of resuspended NEBNext Sample Purification beads (E7104S) were combined with the adapter ligation reaction (40 μL) and mixed by pipetting and incubated on a Hula mixer (rotator mixer) at room temperature for 5 min. Samples were spun and pelleted on a magnet. Supernatant was pipetted off while pellets were retained on a magnet. Beads were combined with 150 μL of Wash Buffer (WSB) (150 μL) and resuspended by flicking the tubes. Tubes were returned to the magnetic rack to allow beads to pellet and supernatant was removed by pipette. The wash step was repeated and the supernatant was removed. Each pellet was resuspended in 21 μL Elution Buffer by gently flicking the tube after removal from the magnetic rack. Each tube was incubated at room temperature for 10 min to allow the elution of RNA. Beads were then pelleted on a magnet until the eluate was clear and colorless. 21 μL, of each eluate was removed and retained in a clean Eppendorf DNA LoBind® tube. 1 μL of RNA was used for concentration measurement using Qubit Assay Kit. Final yield and recovery rate were determined. All 20 μL RNA samples were used for further library prep as described above.

### Example 4C. Coupled Reactions with Soluble Poly(A) Polymerase and Bead Purification (FIG. 1C)

For the coupled reactions approach using soluble enzymes, mix 8 μL quick ligation buffer, 1.2 μL 5 M NaCl solution, 0.5 μL poly(A) polymerase (NEB M0276), 500 ng total RNA, 1.0 μL RT Adaptor (RTA), 6.0 μL RNA Adaptor (RMX) and 3.0 μL T4 DNA ligase (NEB M0202M) supplemented with nuclease-free water to 40 μL in a 0.2 mL thin-walled PCR tube. Incubate the reaction at 37° C. for 20 min followed by 25° C. for 10 min to allow the simultaneous poly(A) tailing and adaptor ligation. Sample purification, RNA concentration determination, and further RNA library prep (using all 20 μL) were carried out as described in Example 4B.

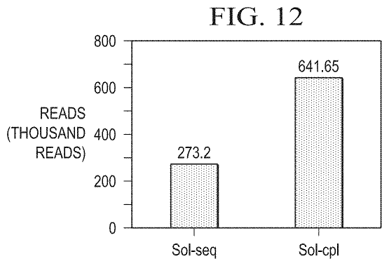

Comparison of Nanopore direct RNA sequencing reads. Each library was prepared using soluble enzyme with bead purification by sequential (Sol-seq) and coupled (Sol-cpl) reaction protocols for poly(A) tailing and adaptor ligation. Results are shown in FIG. 12.

### Example 4D. Sequential Reactions with Immobilized Enzymes (FIG. 1D)

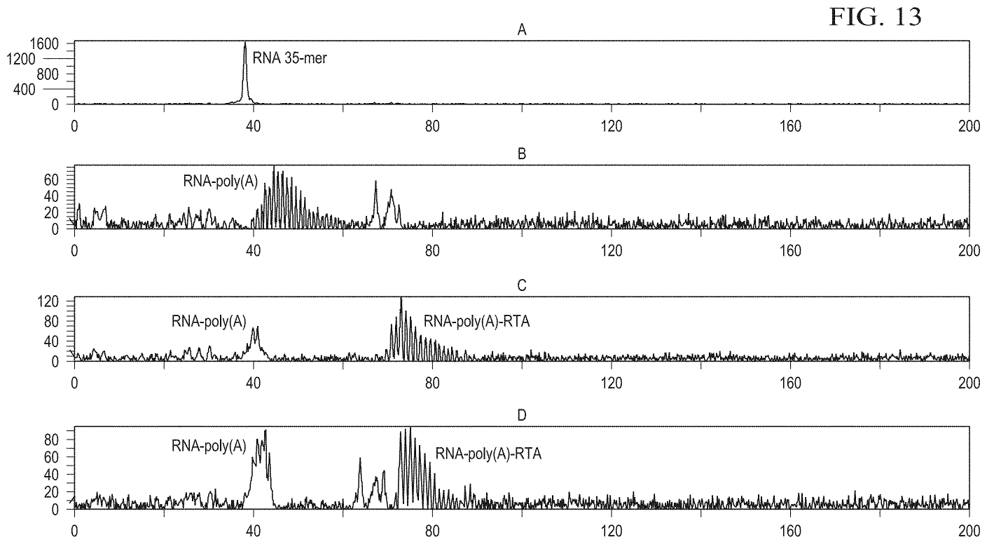

A model study was conducted by CE analysis of sequential treatment of FAM-labeled RNA oligo (35mer) with immobilized poly(A) polymerase and immobilized T4 DNA ligase. Poly(A) tailing was performed at 37° C. for 20 min after mixing 6 μL nuclease-free water, 1 μL 10× poly(A) polymerase reaction buffer (NEB), 1 μL 10 mM ATP, 0.5 μL RNase inhibitor, 1 μL 1 μM RNA 35-mer oligo (100 nM final concentration) and 0.5 μL immobilized poly(A) polymerase (EXAMPLE 1C). Subsequently, after removal of immobilized PAP ligation was carried out at 25° C. for 10 min with the addition of immobilized T4 DNA Ligase (provided by NEB, 60 units/μL) and RTA-poly(dT)15 or RTA-poly(dT)₁₀ (300 nM). Positive results were observed by CE analysis of the samples taken from the poly(A) tailing reaction and adaptor ligation reactions (FIG. 13).

An RNA library was also prepared using immobilized enzymes using the same workflow as described in Example 4B above except that the soluble enzymes (i.e. poly(A) polymerase and T4 DNA ligase) were replaced with their immobilized counterparts. Briefly, poly(A) tailing and ligation were carried out sequentially by incubating RNA with 2.5 μL immobilized Poly(A) polymerase at 37° C. for 20 min., removing the beads, incubating the supernatant with 3.0 μL immobilized T4 DNA ligase at 25° C. for 10 min., and removing the beads with immobilized T4 DNA ligase. Immobilized enzymes, poly(A) polymerase in the first step and T4 DNA ligase in the second step, were separated from the reaction medium on a magnetic rack and the supernatant containing the products and other soluble components were transferred to a fresh tube for the subsequent reaction. No bead purification was performed after the ligation of RTA and RMX adapters. The RNA concentration in the supernatant was determined using Qubit method. A portion of the 40 μL RNA library was supplemented with nuclease-free water to 37.5 μL, and mixed with 37.5 μL RNA running buffer to a final volume of 75 μL before loading into a flow cell for direct RNA sequencing.

FIG. 14 shows that using immobilized enzymes yielded total reads and sequence length comparable to both soluble enzymes and bead purification, indicating that immobilized enzymes can be used to substitute soluble enzymes in catalyzing poly(A) tailing and adaptor ligation reactions. In addition, immobilized enzymes generated many more sequence reads than the soluble enzyme protocol incorporating no bead purification. Thus, removal of the enzyme components from the RNA library appears to be sufficient for generation of high sequence reads in nanopore sequencing presumably by avoiding clogging of nanopores by enzyme molecules. The soluble enzyme protocol without bead purification yielded fewer reads, suggesting that proteins or other components in the reaction mixture may cause nanopore fouling. Soluble enzyme protocol with bead purification also displayed fewer reads probably due to impurities as the result of bead purification.

### Example 4E. Coupled Reactions with Immobilized Enzymes (FIG. 1E)

The sequential poly(A) tailing and ligation steps (shown in Example 4D) were combined into a single, coupled reaction as shown in FIG. 1E. Poly(A) tailing and ligation were carried out by using 2.5 μL immobilized Poly(A) polymerase and 3.0 μL immobilized T4 DNA ligase together at 37° C. for 20 min followed by 25° C. incubation for 10 min. The immobilized enzyme beads were separated from the reaction medium on magnetic rack and the supernatant containing the products and other soluble components were transferred to a fresh tube. A library was also prepared using the sequential reaction protocol with immobilized enzymes described in EXAMPLE 4D.

The RNA concentration in the resulting libraries was determined using Qubit method. The same amount of RNA from each library was used to prepare the sequencing mixtures supplemented with nuclease-free water to a volume of 37.5 μL and another 37.5 μL of RNA running buffer (RRB) were used to prepare 75 μL sample for RNA sequencing according to Example 2D. Direct RNA sequencing for both sequential (Example 2C) and coupled reaction (this example) were performed on a MinION® MkIb with R9.4 flow cells as introduced before.

Results shown in FIG. 15 contrast the number of sequencing read from the coupled reaction protocol to that of the sequential reaction protocol using immobilized enzymes. The library prepared using a sequential reaction strategy (Example 4D) with an RNA sequencing input of 105 ng of RNA yielded 459 K reads, which is comparable to the results presented in Example 4C with final yield of 488.5 K reads from 164.4 ng of RNA input. However, the library prepared in this Example using a coupled reaction protocol with immobilized enzymes with similar amount of RNA input produced almost a 3-fold increase in RNA sequencing reads compared to a sequential reaction protocol using immobilized enzymes.

### Example 4F. Coupled Reactions with Co-Immobilized Enzymes

O⁶-benzylguainine (BG) functionalized magnetic beads coated with PEG₇₅₀ (100 of a 25% (v/v) slurry) were used for enzyme co-immobilization. Poly(A) polymerase—SNAP fusion protein and T4 DNA ligase—SNAP fusion protein (12.5 μg of each) were dissolved in 125 μL buffer (1×PBS with 300 mM NaCl), combined with the washed BG beads, and incubated at 4° C. overnight to immobilize the fusion protein on the beads, according to the procedure described in EXAMPLE 1C. The co-immobilized beads were washed with the same buffer 8 times to remove any unbound enzyme molecules. Diluent A buffer without BSA (NEB) with 100 mM NaCl was used to resuspend the beads with immobilized fusion protein for storage at −80° C.

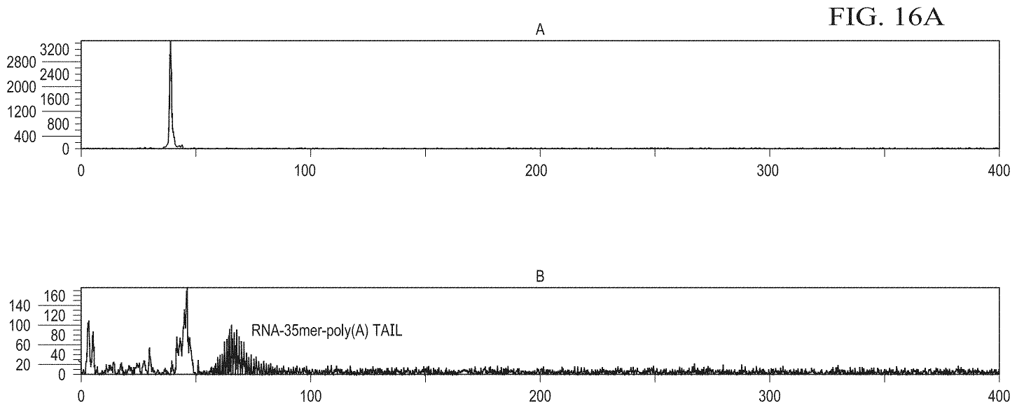

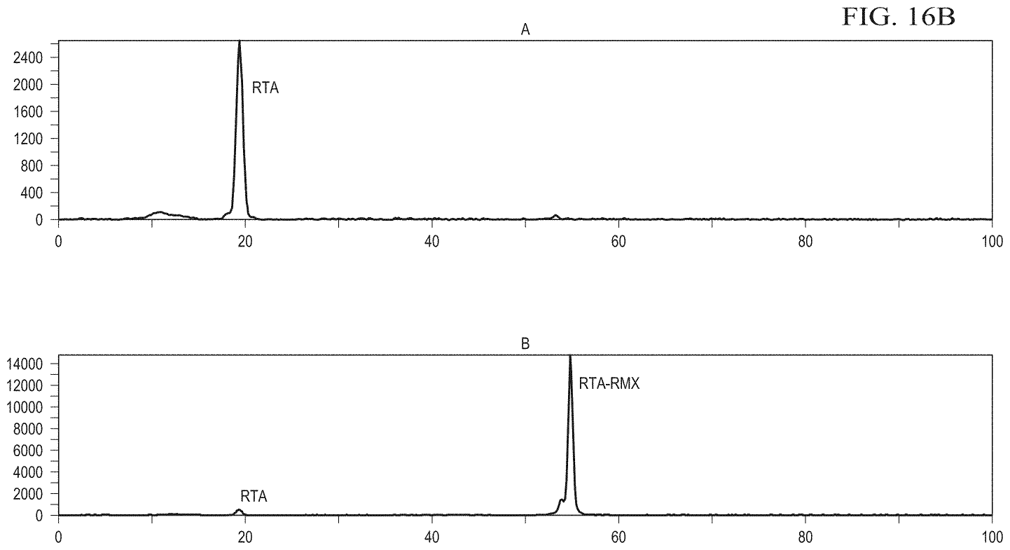

Poly(A) tailing and adaptor ligation activities were measured for the bead sample co-immobilized with poly(A) polymerase and T4 DNA ligase. 5 μL of the co-immobilized enzyme bead mixture were used to replace 2.5 μL immobilized poly(A) polymerase and 3 immobilized T4 DNA ligase in each activity assay. The co-immobilized enzymes displayed both poly(A) polymerase activity (FIG. 16A) and T4 DNA ligase activity (FIG. 16B).

### Example 5: Automated Library Construction and Sequencing

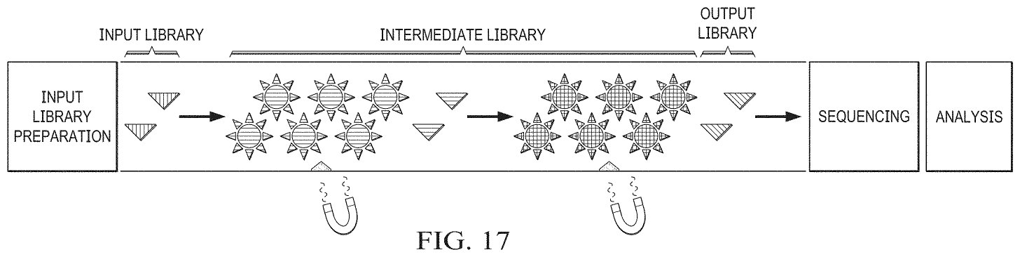

While next-generation sequencing (NGS) has greatly advanced biological research and clinical diagnostics, the process would benefit from automation from library construction to sequencing libraries and data analysis. The preceding examples demonstrate that application of immobilized enzymes in multi-reaction library construction workflows avoids bead based nucleic acid purification which may cause sample loss and bias in fragment distribution. Combining the single-reaction preparation of Example 4 with NGS (e.g., using robotics and/or microfluidics) advances automation in sequencing. Specifically, otherwise cumbersome steps of adding adapters to nucleic acid libraries to be sequenced may be in a single reaction vessel and fed directly into sequencing platforms. The concomitant reduction in handling will reduce error rates and variations in high-throughput research and clinical application of Nanopore and other NGS technologies (FIG. 17).

As shown, magnetic beads bearing enzymes are positioned in proper enclosed chamber and tunnel to process an input or intermediate library. There is no extra purification step required for separation of the enzymes and the resulting products, between or after enzymatic reaction steps. The input library can be produced by a method, for example, RNA extraction, that incorporates a properly designed automated workflow. An output library can be properly formulated for direct sequencing on a nanopore sequencing device, i.e. flow cell, such as currently available R9.4.1. or R10. Ultimately, this workflow is linked to locally based or cloud-based computer software to provide a fully automated sequencing solution.

### Example 6: Direct RNA Sequencing with Low Input RNA is Possible when Immobilized Enzymes are Used in Library Prep

All sequencing reads using the libraries described in Example 4 were performed with 500 ng of RNA as suggested by Oxford Nanopore Technologies. This example demonstrates construction and successful sequencing of duplicate libraries from a lower initial input (100 ng) of Listeria monocytogenes RNA using either a sequential or coupled reaction protocol with immobilized poly(A) polymerase and T4 DNA Ligase. For sequential reaction protocol, 3 μL quick ligation buffer, 0.45 μL 5 M NaCl solution, 1.5 μL immobilized poly(A) polymerase, and 100 ng total RNA, supplemented with nuclease-free water to 10 μL were mixed in a 0.2 mL thin-walled PCR tube and incubated at 37° C. for 20 min for poly(A) tailing. After immobilized poly(A) polymerase beads were removed by placing the tube on the magnetic rack, 0.5 μL RT Adaptor (RTA), 3.0 μL RNA Adaptor (RMX) and 1.5 μL immobilized T4 DNA ligase were added to the poly(A) tailed RNA sample to yield a final volume of 15 μL. This mixture was incubated at 25° C. for 10 min for adaptor ligation. The immobilized ligase was removed using the magnet. For the coupled reaction protocol, the same amounts of enzymes and buffer were utilized as disclosed above (except that all the components were combined in a single tube). The mixtures were incubated at 37° C. for 20 min and 25° C. for 10 min, consecutively, and both immobilized enzymes were removed in a single step on the magnetic rack. The RNA yields of the prepared libraries were determined by high sensitivity RNA Qubit assay.

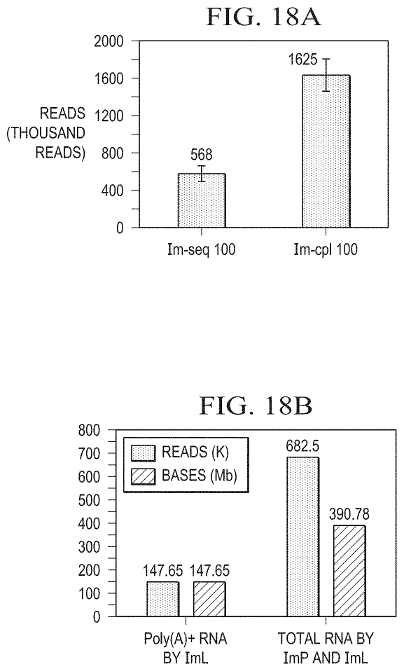

With the initial RNA input of 100 ng, the sequential and coupled reaction methods resulted in recovery of an average of 38 ng and 83 ng, respectively, from duplicate libraries. Thus, the recovery rate for RNA library prep using the coupled reaction protocol is 83% of the initial input, much higher than that of the sequential reaction protocol (FIG. 18A). The results also suggest that a coupled reaction process generates more reads compared to the sequential reaction. In addition, a sequential reaction generates 568 K reads on average, higher than that obtained from the protocol using soluble enzymes with bead purification. The results demonstrate that low input RNA library construction can be achieved by using both sequential and coupled reaction protocols with immobilized enzymes (FIG. 18A).

Similar results were obtained with library construction from a low initial input mammalian human brain RNA (100 ng). FIG. 18B shows direct RNA sequencing of low input of poly(A) tailed human RNA (100 ng PolyA+ RNA) prepared by ligation to RTA and RMX adaptors with immobilized T4 DNA Ligase (ImL). Furthermore, total mammalian RNA library (100 ng input), prepared with immobilized poly(A) polymerase and immobilized T4 DNA ligase (ImP and ImL) using the coupled reaction protocol described above in this example, was successfully used for direct RNA sequencing.

### Example 7: Metrics of the Ont Direct RNA-Seq Datasets from Various Library Preparation Protocols

Duplicate libraries using each of the five RNA library preparation protocols were made with 500 ng input RNA extracted from Listeria monocytogenes (ATCC 1115) culture.

The final volume for the enzymatic reactions was 40 μl prior to bead purification; for soluble enzyme protocols, Sol-seq and Sol-cpl, each RNA library was further purified with RNA binding beads and eluted in 20 μl volume. For immobilized enzyme protocols enzyme removal was performed without purification using RNA binding beads prior to use of a fraction of the library sample for Nanopore direct RNA sequencing. Results are shown in TABLE 1.

TABLE 1

| 500 ng | | | | | | | | | | | | |
|---|---|---|---|---|---|---|---|---|---|---|---|---|
| input | | | | | | | | | | | | |
| libraries | Sol-seq | | | | Sol-cpl | | | | Im-seq | | | |
| Replicate | R1 | R2 | Avg | Std | R1 | R2 | Avg | Std | R1 | R2 | Avg | Std |
| | FAM9 | FAM9 | | | FAM9 | FAN2 | | | FAN2 | FAN2 | | |
| | 6082 | 4718 | | | 4789 | 3186 | | | 7746 | 7699 | | |
| Recovery | 29% | 39% | 34% | | 34% | 34% | 34% | | 31% | 47% | 39% | |
| rate | | | | | | | | | | | | |
| Loading | 176 | 197 | 186 | | 196 | 222 | 209 | | 88 | 116 | 102 | |
| (ng) | | | | | | | | | | | | |
| Loading | 20 | 20 | | | 20 | 20 | | | 20 | 20 | | |
| (μL) | | | | | | | | | | | | |
| Reads | 134 | 149 | 141 | 8 | 642 | 757 | 699 | 58 | 903 | 798 | 850 | 52 |
| generated | | | | | | | | | | | | |
| (K) | | | | | | | | | | | | |
| Bases | 136 | 168 | 152 | 16 | 681 | 816 | 748 | 68 | 836 | 737 | 786 | 50 |
| generated | | | | | | | | | | | | |
| (Mb) | | | | | | | | | | | | |
| Estimated | 157 | 200 | 178 | 21 | 829 | 962 | 895 | 66 | 1030 | 896 | 963 | 67 |
| generated | | | | | | | | | | | | |
| (Mb) | | | | | | | | | | | | |
| Bases/ | 0.86 | 0.84 | 0.85 | | 0.82 | 0.85 | 0.84 | | 0.81 | 0.82 | 0.82 | |
| Estimated | | | | | | | | | | | | |
| Run | 18 | 22 | 20 | | 45 | 53 | 49 | | 40 | 40 | 40 | |
| Length | | | | | | | | | | | | |
| (hr) | | | | | | | | | | | | |

| 500 ng | | | | | | | | |
|---|---|---|---|---|---|---|---|---|
| input | | | | | | | | |
| libraries | Im-cpl | | | | Sol w/o BP | | | |
| Replicate | R1 | R2 | Avg | Std | R1 | R2 | Avg | Std |
| | FAM9 | FAN0 | | | FAL6 | FAL7 | | |
| | 4697 | 3228 | | | 8474 | 0280 | | |
| Recovery | 100% | 117% | 108% | | | | | |
| rate | | | | | | | | |
| Loading | 176 | 197 | 186 | | | | | |
| (ng) | | | | | | | | |
| Loading | 11 | 15.6 | | | | | | |
| (μL) | | | | | | | | |
| Reads | 1580 | 1070 | 1325 | 255 | 430 | 226 | 328 | 102 |
| generated | | | | | | | | |
| (K) | | | | | | | | |
| Bases | 1610 | 1120 | 1365 | 245 | 427 | 146 | 286 | 140 |
| generated | | | | | | | | |
| (Mb) | | | | | | | | |
| Estimated | 2000 | 1390 | 1695 | 305 | 526 | 192 | 359 | 167 |
| generated | | | | | | | | |
| (Mb) | | | | | | | | |
| Bases/ | 0.81 | 0.81 | 0.81 | | 0.81 | 0.76 | 0.80 | |
| Estimated | | | | | | | | |
| Run | 72 | 72 | 72 | | 43 | 21 | 32 | |
| Length | | | | | | | | |
| (hr) | | | | | | | | |

### Example 8: Comparison of the Major Metrics of the Ont Direct RNA-Seq Datasets from Various Library Preparation Methods

The average of the major metrics from duplicate libraries prepared using each of the five RNA library preparation protocols were analyzed. Each library was made with 500 ng input RNA extracted from Listeria monocytogenes (ATCC 1115) culture. The final volume for the enzymatic reactions was 40 μl prior to bead purification; for soluble enzyme protocols, Sol-eq and Sol-cpl, each RNA library was further purified with RNA binding beads and eluted in 20 μl volume. For immobilized enzyme protocols enzyme removal was performed without purification using RNA binding beads prior to use of a fraction of the library sample for Nanopore direct RNA sequencing.

Averages of two replicate runs are shown in TABLE 2.

TABLE 2

| 500 ng input libraries | Sol-seq | Sol-cpl | Im-seq | Im-cpl |
|---|---|---|---|---|
| Recovery rate | 0.34 | 0.34 | 0.39 | 1.08 |
| Loading (ng) | 186.4 | 208.8 | 102.0 | 186.4 |
| Loading (μL) | 20 | 20 | 20 | 13.3 |
| Reads generated (K) | 141.42 | 699.33 | 850.42 | 1325 |
| Bases generated (Mb) | 151.58 | 748.15 | 786.34 | 1365 |
| Estimated | 178.2 | 895.4 | 963.11 | 1695 |
| generated (Mb) | | | | |
| Bases/Estimated | 0.85 | 0.84 | 0.82 | 0.81 |
| Run Length (hr) | 20.2 | 48.8 | 40.3 | 72.0 |
| Mean read length (nt) | 1145.3 | 1156.4 | 1065.9 | 1107.9 |
| Median read | 1157 | 1028 | 950 | 951 |
| length (nt) | | | | |
| Read length N50 | 1493 | 1520 | 1464 | 1511 |
| Mean read quality | 10.2 | 10.1 | 10.2 | 10.2 |
| Median read quality | 10.4 | 10.2 | 10.4 | 10.3 |
| Mapping | 99.6% | 99.2% | 99.3% | 99.1% |
| Mapped reads | 140.84 | 693.52 | 844.55 | 1313.08 |
| Expected Reads | 141.4 | 699.3 | 1615.8 | 3902.4 |
| (K)/Library | | | | |
| Expected bases | 151.6 | 748.2 | 1494.0 | 4020.2 |
| (Mb)/Library | | | | |
| Ratio of Expected | 1 | 4.9 | 11.4 | 27.6 |
| Reads (K)/Library | | | | |

### Example 9: Comparison of Nanopore Direct RNA Sequencing Reads

Duplicate libraries were prepared using the five protocols illustrated in FIGS. 1A-1E. Each library was made with 500 ng input RNA extracted from Listeria monocytogenes (ATCC 1115) culture. For Sol-seq and Sol-cpl, each RNA library was further purified with RNA binding beads prior to Nanopore sequencing. Both immobilized enzyme protocols did not utilize a purification step with RNA-binding beads after enzymatic treatment, and enzyme removal was performed with magnetic rack prior to sequencing. Results are shown in FIG. 19.

### Example 10: Reduced Inactivation of Nanopores Using Libraries Prepared with Immobilized Enzymes

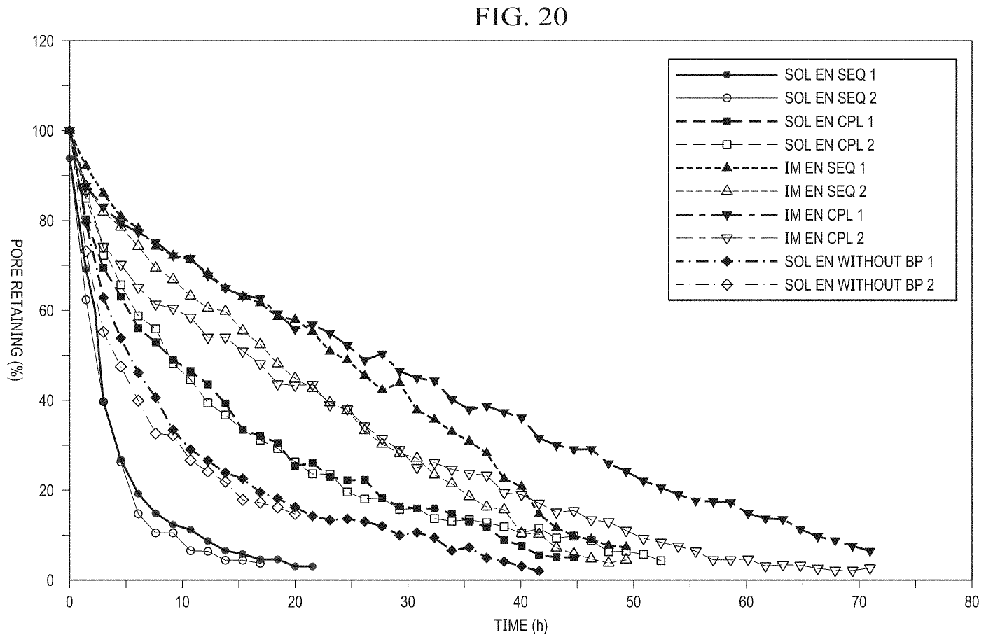

Data presented in Examples 7-9 demonstrates that bead purification following library preparation with soluble enzymes is associated with a considerable loss of library RNA. Example 13 shows further that omitting a bead purification step (to avoid this loss of RNA) may not be a favorable solution in that the resulting libraries produce substantially fewer sequence reads from the same amount of input RNA compared to the immobilized enzyme protocols. A possible reason for fewer sequence reads from the libraries produced by the soluble enzyme protocol without bead purification is that residual polymerase and/or ligase may occlude nanopores. On the other hand, bead purification may affect library quality due to loss of RNA (or certain RNA types) and may also produce impurity derived from the wash solutions. In this example, the activity of nanopores was monitored over the course of a sequencing run from MinKNOW® report. Results shown in FIG. 20 demonstrate that more nanopores remain active (upper trace) when the libraries were prepared with immobilized enzymes compared to those prepared with soluble enzymes. For example, after two hours of sequencing, about 90% of the pores processing immobilized enzyme libraries remained active while only about 65% of the pores processing soluble enzyme libraries remained active. At 8 hours, about half of the pores processing immobilized enzyme libraries remained active while only about 10% of the pores processing soluble enzyme libraries remained active. These results demonstrate that the use of immobilized enzymes in library construction can increase nanopore sequencing output, possibly by reducing nanopore fouling.

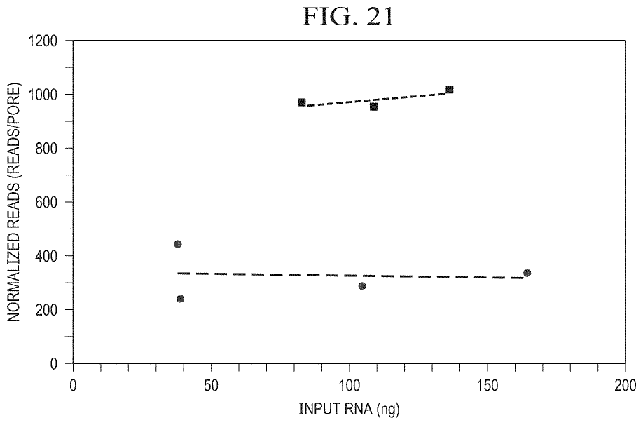

The number of reads per pore was also evaluated for the coupled reaction method. Normalized reads shown in FIG. 21 were generated from dividing the reads from sequencing a Listeria RNA library by the number of pores.

Three Listeria RNA libraries were prepared using the coupled reaction protocol using immobilized enzymes (squares). Two low input RNA libraries were prepared in 15 uL following the coupled reaction protocol as described in Example 5, resulting in direct RNA sequencing of 83 ng and 136.5 ng RNA per flow cell, respectively. The third library was prepared as described in Example 4E with 500 ng input RNA in 40 uL and only part of the resulting library (109 ng) was loaded for sequencing.

The number of reads per pore was also examined for a set of four libraries prepared using the sequential reaction protocol using immobilized enzymes (dots). Two low input RNA libraries were prepared in 15 uL as described in Example 5, resulting in sequencing 38 ng and 39 ng RNA per flow cell, respectively. Two 500 ng input RNA libraries were made in 40 uL as described in Example 4D and a portion of each resulting library, 105 ng and 164.4 ng, respectively, was used for loading on a flow cell and direct RNA sequencing.

Results indicate that the coupled reaction protocol can generate a significantly higher reads per nanopore compared to the sequential reaction protocol using the same set of immobilized enzymes and conditions (i.e. buffer, total reaction time and volume).

### Example 11: DNA Library Construction Workflow for Nanopore Sequencing of Ultra-Long Templates without Bead Purification

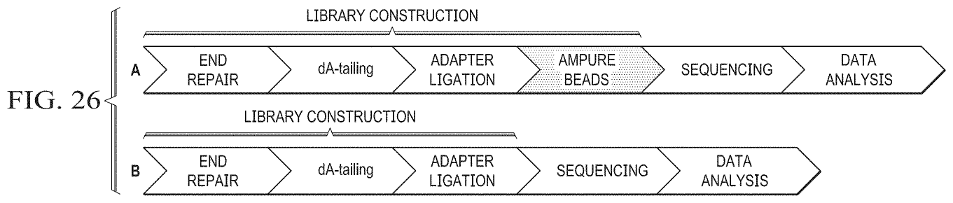

This example describes a new strategy for preparation of DNA libraries for nanopore DNA sequencing. The current ONT protocol, as depicted in FIG. 26 and Example 13, utilizes a set of four DNA-modifying enzymes to perform end-polishing, dA-tailing and adaptor ligation, in conjunction with bead purification to produce a library for long-read sequencing. Addition of a single 3′A in dA-tailing demands use of PEG in the subsequent adaptor ligation because T/A pairing is inefficient in the absence of PEG or other enhancers. However, use of PEG in conjunction with use of AMPure® beads may not be ideal since PEG can cause DNA compaction onto beads. In addition, application of bead purification can result in shearing of long DNA templates thereby adversely affecting the ability of ultra-long sequence reads by nanopore sequencing.

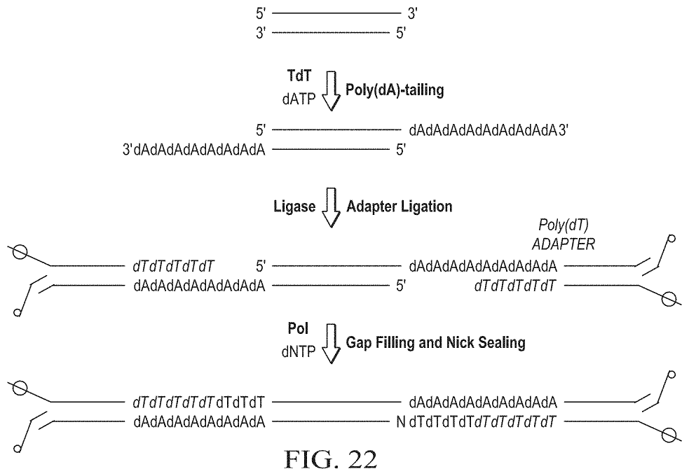

A new method is proposed here and illustrated in FIG. 22. As shown, the proposed method does not use bead purification and/or may include or exclude use PEG during DNA library preparation. This workflow is comprised of three major enzymatic steps. First, Terminal deoxynucleotidyl transferase (TdT) is employed to catalyze poly(dA) tailing at 3′ end of DNA fragments possibly pre-treated with end-polishing enzyme(s). The oligo(dA) overhang can then efficiently ligate with an adaptor with a 3′ Poly(dT) overhang and motor protein, in the presence or absence of PEG in the reaction medium. Next, gap-filling and nick sealing can be accomplished with DNA polymerase and DNA Ligase. Enzymes may be removed, inactivated or present in the final sequencing library. Breakage of DNA molecules may be reduced and/or recovery of long DNA templates may be improved by avoiding use of bead purification and/or PEG. As practitioners having the benefit of this disclosure will appreciate, TdT can add other types of oligos, such as poly(dT) or poly(dG) to be suitable for other adaptor ligation strategies in the absence of PEG or DNA-compacting factor. Thus, different enzymes and tailing approaches can be designed to prepare DNA-adaptor molecules anchored with motor protein and other features required for nanopore sequencing.

### Example 12: DNA Library Preparation

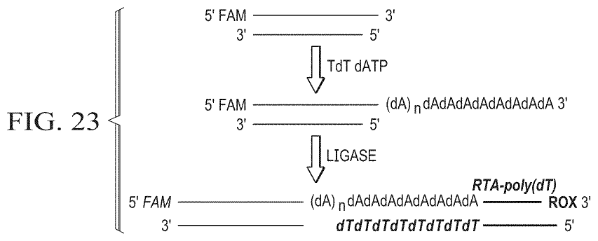

This example demonstrates poly(dA) tailing of a synthetic DNA substrate and subsequent ligation of the products possessing various lengths of 3′ poly(dA) sequences to an adaptor having a 3′ poly(dT) overhang as illustrated in FIG. 23.

### Example 6A. Poly(dA)-Tailing Mediated by Terminal Deoxynucleotidyl Transferase (TdT)

This example demonstrates poly(dA) tailing of a synthetic DNA substrate by Terminal deoxynucleotidyl Transferase (TdT). A double-stranded DNA substrate was formed by annealing two oligonucleotides, with one possessing a 5′ fluorophore probe, FAM and 3′ protruding overhang for addition of Poly(dA) tails. 5′FAM-labeled double-stranded DNA was treated with TdT in the presence of various concentrations of dATP to create different substrate to dATP ratios (e.g. 1:100 and 1:200). CE analysis was performed to assess the incorporation of dAMP at the 3′ termini of 5′FAM-labeled DNA strand and estimation of the lengths (or range) of poly(dA) tails.

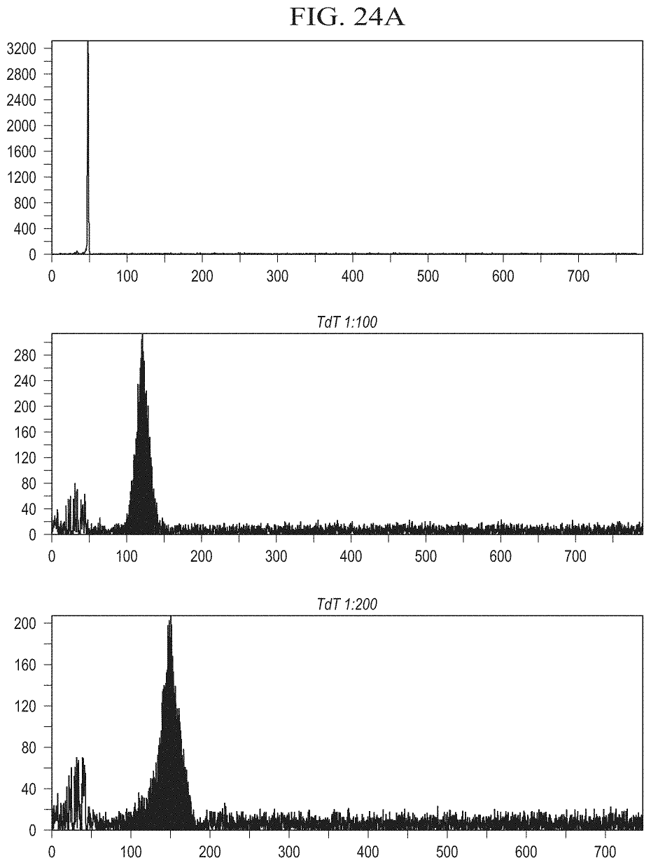

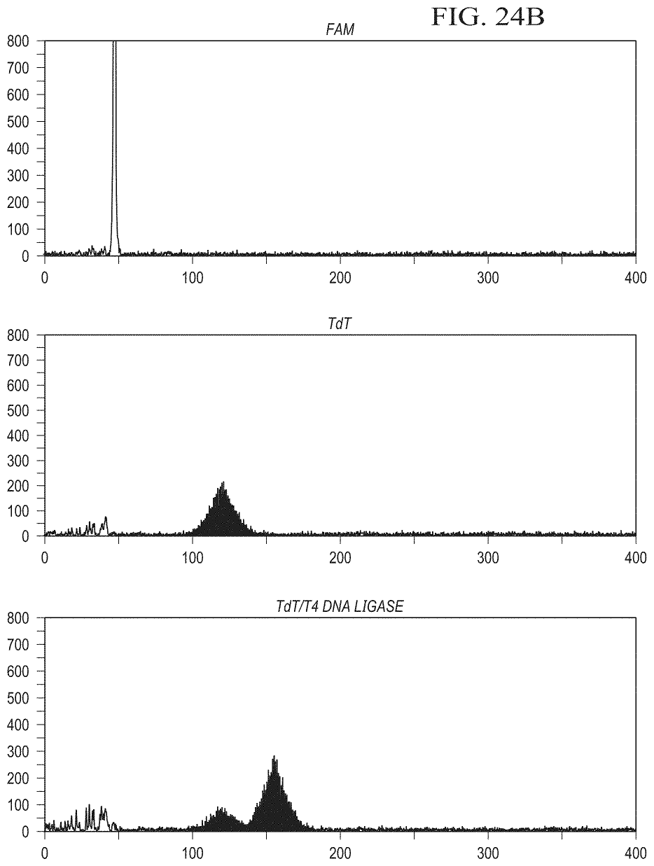

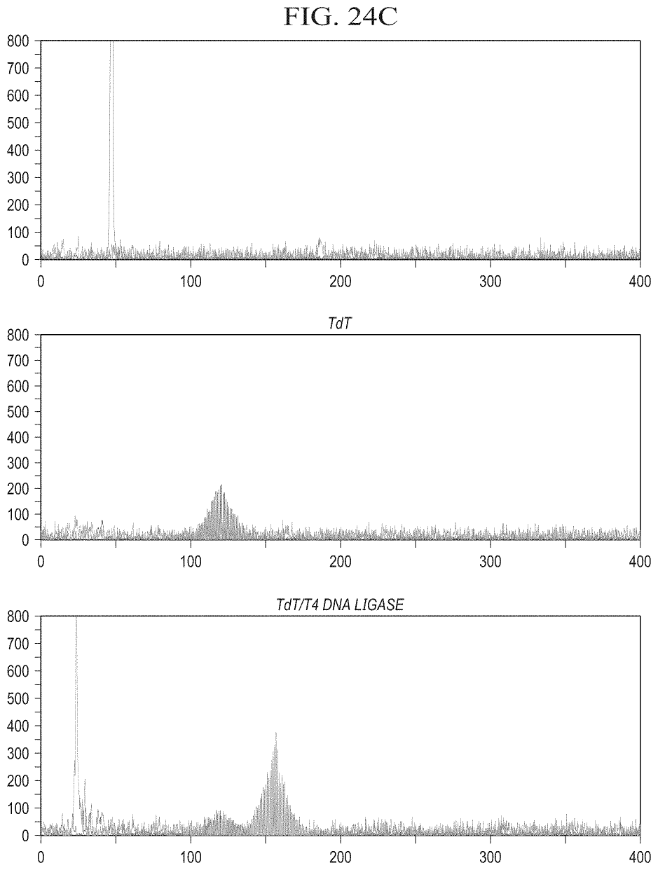

3′ poly(dA) tailing was carried out in a 30 μl reaction volume in the presence of 0.1 μM of the DNA substrate, 0.5 μl (20 units) of TdT (NEB, M0315S, 40,000 units/ml,), 1×TdT Reaction Buffer (NEB), 0.25 mM CoCl₂ and 10 or 20 μM of dATP. The reactions were performed at 37° C. for 30 min, followed by treatment at 70° C. for 10 min in a T-100 Thermocycler (Bio-Rad Laboratories, Hercules, Calif.). The reactions were terminated by diluting in 1:1 ratio in 50 mM EDTA and 0.1% Tween-20, and analyzed by CE technique and Peak Scanner software. Results shown in FIG. 24A demonstrate that poly(dA) tailing can be performed in 1×TdT buffer supplemented with CoCl₂. Efficient conversion of the DNA substrate to poly(dA) tailed products of various lengths was observed after treatment with TdT and the length of the poly(dA) can be modulated by the control of substrate-to-dATP ratio.

### Example 12B. Sequential Poly(dA) Tailing and Adaptor Ligation

The FAM-labeled double-stranded DNA substrate was assayed for sequential poly(dA) tailing with soluble TdT as described in EXAMPLE 12A and adaptor ligation with soluble T4 DNA Ligase.

A modified RTA adaptor, RTA-Poly(dT) was made by annealing two oligonucleotides, derived from the sequences of RTA (provided by ONT), with one oligonucleotide containing 5′ phospho group and 3′ ROX probe, and the second one being modified to possess 3′ poly(dT). 3′ poly(dA) tailing was carried out in a 30 μl reaction volume in the presence of 0.1 μM of the DNA substrate, 1 unit of Terminal Deoxynucleotide Transferase (NEB, M0315S, 40,000 units/ml,), 1×TdT Reaction Buffer (NEB), 0.25 mM CoCl₂ and 10, 20 or 50 μM of dATP. The reactions were performed at 37° C. for 30 min, followed by treatment at 70° C. for 10 min in a T-100 Thermocycler (Bio-Rad Laboratories, Hercules, Calif.). Next, adaptor ligation was performed at 25° C. for 30 min after addition of 100 nM RTA-poly(dT), 1 mM ATP and 1 μl of T4 DNA Ligase (NEB, M0202S, 400,000 units/0 to the TdT-treated samples. The reactions were terminated by diluting in 50 mM EDTA and 0.1% Tween-20, and analyzed by CE technique and Peak Scanner software. Efficient conversion of the DNA substrate to poly(dA) tailed products of various lengths was observed after treatment with TdT. Joining of poly(dA) tailed products and modified RTA was also detected because of the shift of the FAM labeled products and the co-localization of FAM and ROX signals after ligation reaction, in comparison with the TdT-treated sample.

FIG. 24B shows a peak formed by a range of FAM-labeled products (in dark gray), representing various 3′ poly(dA) lengths. Subsequently, the reaction medium containing the poly(dA)-tailed DNA products, was incubated with T4 DNA ligase and RTA-poly(dT) adaptor possessing 3′ poly(dT) and 5′ ROX. FIG. 24C shows co-localization of the fluorescence signals of FAM (dark gray) and ROX (light gray) indicates ligation of the 5′FAM-labeled DNA species to the 3′ ROX-labeled strand of the adaptor. Successful ligation also resulted in a shift of the FAM-labeled species (major peak in TdT sample) to higher molecular products (major Peak in TdT/T4 DNA Ligase sample).

Results shown FIG. 24B and FIG. 24C demonstrate that both poly(dA) and ligation reactions can be performed in 1×TdT buffer supplemented with CoCl₂.

### Example 12C. Ligation of Poly(dA) tailed DNA with Adaptor with Soluble and Immobilized Ligase

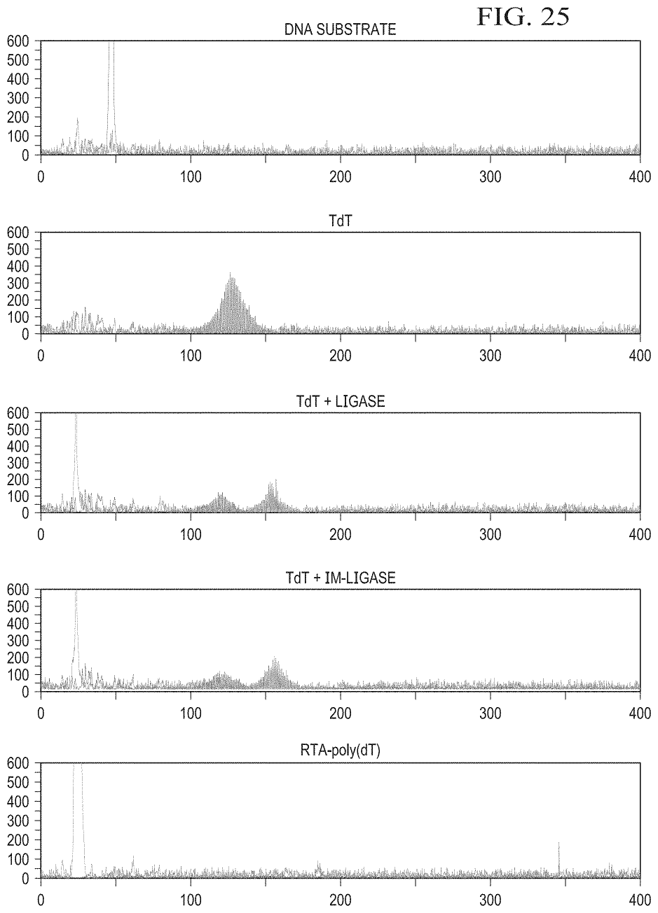

As shown in FIG. 25, a FAM-labeled double-stranded DNA substrate was first tailed using soluble TdT as described in EXAMPLE 6A and then ligated to an adapter with either soluble or immobilized T4 DNA Ligase. 3′ poly(dA) tailing was carried out in a 30 μl reaction volume in the presence of 0.1 μM of the DNA substrate, 1 unit of Terminal Deoxynucleotide Transferase (NEB, M0315S, 40,000 units/ml,), 1×TdT Reaction Buffer (NEB), 0.25 mM CoCl₂ and 10, 20 or 50 μM of dATP. The reactions were performed at 37° C. for 30 min, followed by treatment at 70° C. for 10 min in a T-100 Thermocycler (Bio-Rad Laboratories, Hercules, Calif.). Next, adaptor ligation was performed at 25° C. for 30 min after addition of 100 nM RTA-poly(dT), 1 mM ATP and 1 μl of T4 DNA Ligase (NEB, M0202S, 400,000 units/μl) or immobilized T4 DNA Ligase (NEB, Production Lot 1, 60 units/μl) to the TdT-treated samples. The reactions were terminated by diluting in 50 mM EDTA and 0.1% Tween-20, and analyzed by CE technique and Peak Scanner software. Efficient conversion of the DNA substrate to poly(dA) tailed products of various lengths was observed after treatment with TdT. Poly(dA) tailed products and modified RTA were detected with either soluble or immobilized T4 DNA ligase because of the shift of the FAM labeled products and the co-localization of FAM and ROX signals after ligation reaction, in comparison with the TdT-treated sample. These results show that both poly(dA) and ligation reactions can be performed in 1×TdT buffer supplemented with CoCl₂.

### Example 13: DNA Library Construction Using Immobilized DNA Modifying Enzymes

Many existing methods rely on steps (e.g., AMPure® bead purification) that shear long DNA molecules and are detrimental to long-read sequencing. In addition to use for RNA library preparation for sequencing, immobilized enzymes can be used to construct DNA libraries. FIG. 26 shows a schematic of DNA library construction using a set of four immobilized DNA modifying enzymes (IM-T4 DNA polymerase IM-T4 PNK, IM-Taq DNA Pol, IM-T4 DNA Ligase). Soluble forms of these enzymes are currently used for Nanopore DNA library construction. The following example sets forth the use of relevant immobilized enzymes to generate a DNA library by using an oligo DNA model system with a CE technique to conduct step-by-step analyses.

### Example 14: DNA LIBRARY CONSTRUCTION USING IMMOBILIZED DNA Modifying Enzymes

A DNA library construction protocol for nanopore sequencing may include fragmentation, end repair (blunting and 5′ phosphorylation), 3′ A-tailing and adaptor ligation. Once the sample DNA has been sheared, the fragment ends are repaired by blunting and 5′ phosphorylation with a mixture of enzymes, such as T4 polynucleotide kinase (PNK) and T4 DNA polymerase (T4 DNA pol). This end repair step is followed by 3′ A-tailing at 37° C. using a mesophilic polymerase such as Klenow Fragment 3′-5′ exonuclease minus¹¹, or at elevated temperatures using a thermophilic polymerase such as Taq DNA polymerase (Taq DNA pol) (Head, S. R. et al. Library construction for next-generation sequencing: overviews and challenges. BioTechniques 56, 61-64, 66, 68, passim (2014); Star, B. et al. Palindromic Sequence Artifacts Generated during Next Generation Sequencing Library Preparation from Historic and Ancient DNA. PLOS ONE 9, e89676 (2014)). 3′ A-tailed DNA fragments are ligated to an adaptor using a T/A ligation method and purified using AMPure® beads prior to nanopore sequencing. Bead-based purification step(s) may result in shearing large DNA which is detrimental to long read sequencing. In addition, T/A ligation efficiency is highly dependent on the presence of crowding agent, such as PEG, however, use of a crowding agent, namely PEG, appears to cause large DNA molecules to compact (Warren M. Mardoum, Stephanie M. Gorczyca, Kathryn E. Regan, Tsai-Chin Wu, and Rae M. Robertson-Anderson. Crowding Induces Entropically-Driven Changes to DNA Dynamics That Depend on Crowder Structure and Ionic Conditions. Front Phys. 2018; 6: 53; Heikki Ojal, Gabija Ziedait, Anders E. Wallin, Dennis H. Bamford, Edward Hæggström. Optical tweezers reveal force plateau and internal friction in PEG-induced DNA condensation. European Biophysics Journal, March 2014, Volume 43, Issue 2-3, pp 71-79). Consequently, the PEG-induced DNA compaction may reduce DNA elution from AMPure® beads, resulting in low library yield of large DNA.

An enzyme immobilization strategy was previously utilized to perform DNA library construction for the sequencing on the Illumina platform (Zhang, Aihua, et al. Solid-phase enzyme catalysis of DNA end repair and 3′ A-tailing reduces GC-bias in next-generation sequencing of human genomic DNA. Scientific reports 8.1 (2018): 1-11.). The relevant DNA-modifying enzymes were produced as SNAP-tagged fusion proteins and immobilized by covalent conjugation onto magnetic beads functionalized with benzyl guanine ligand (the substrate of SNAP-tag). These immobilized enzymes were successfully applied to Illumina DNA library construction in place their soluble counterparts. One of the major of the major advantages is that the enzymes can be removed without heat treatment or AMPure® bead purification.

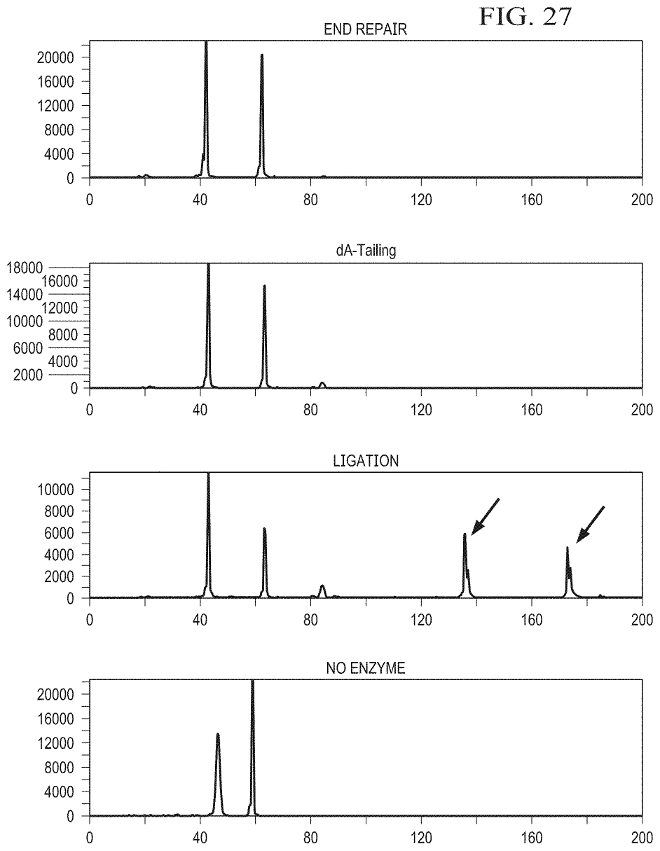

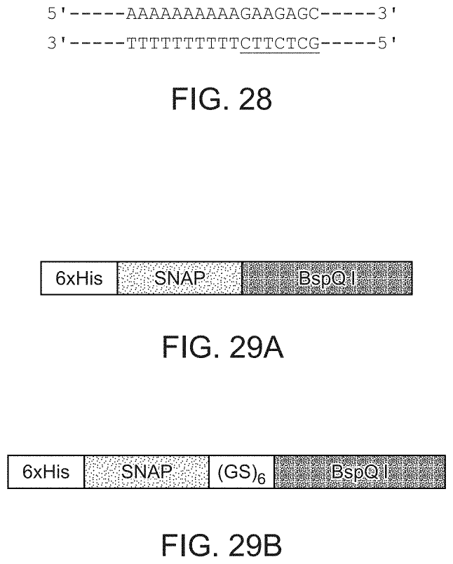

This example demonstrates that the same set of enzymes can be used for the current workflow of Nanopore DNA library construction. In this example, each enzymatic reaction step was monitored by fluorescence capillary gel electrophoresis (CE) using a synthetic double stranded DNA end-labeled with a fluorescent probe, FAM. DNA was end-repaired for 30 min at 20° C. using immobilized T4 DNA pol and T4 polynucleotide kinase in a 20 μl reaction in the presence of 1×NEBNext End Repair Buffer II. These end-repair enzymes were pelleted on a magnetic rack and the supernatant was transferred to a new tube for 3′ A-tailing with immobilized Taq DNA pol at 37° C. for 30 min. The resulting product was ligated to an adaptor with an end possessing 5′ phospho group and 3′T. The reactions were terminated by diluting in 50 mM EDTA and 0.1% Tween-20. The reactions were performed in a T-100 Thermocycler (Bio-Rad Laboratories, Hercules, Calif.) and analyzed by CE technique and Peak Scanner software. A partial ligation of the DNA substrate to the adaptor is shown in FIG. 27, indicating that T/A ligation can be performed in 1×NEBNext End Repair Buffer II without supplement with PEG.

### Example 15: Recombinant Plasmids for SNAP-Tagged BspQI Expression

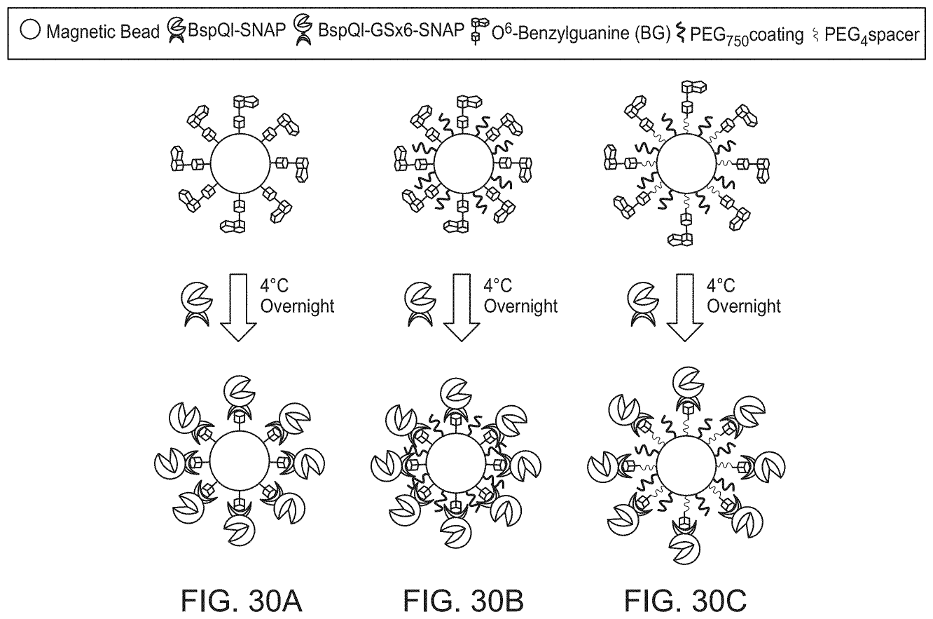

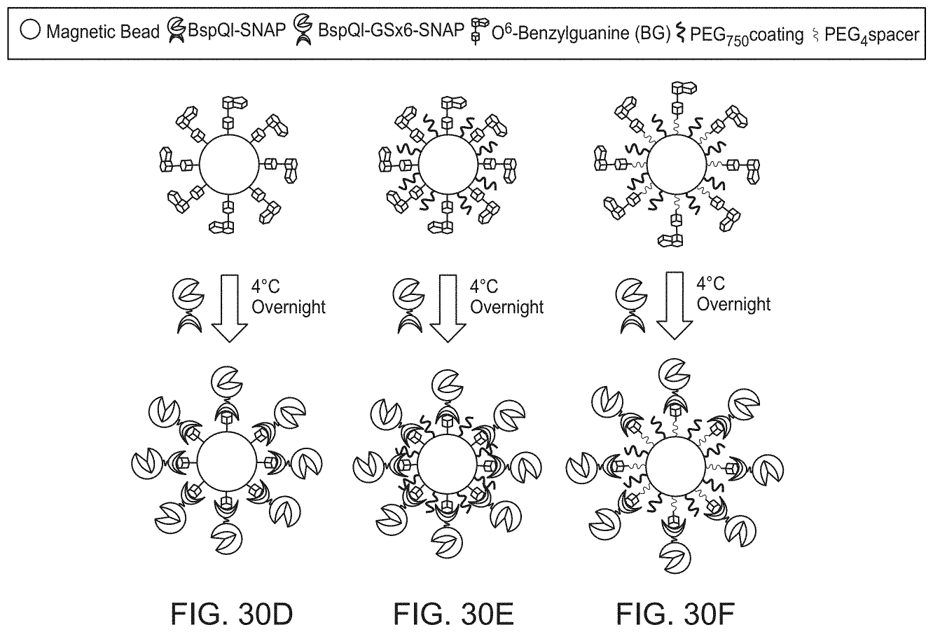

As disclosed herein, the microenvironment of an immobilized enzyme may differ from a corresponding soluble enzyme, for example, with respect to the spatial orientation and flexibility of the enzyme—differences that may impact enzymatic activity. SNAP-tagged BspQI fusion constructs were made without (FIGS. 30A-30C) or with (FIGS. 30D-30F) a flexible peptide linker (GSx6) for protein production and immobilization.

Two E. coli plasmid constructs were constructed to express SNAP-tagged BspQI fusion proteins. Both constructs contain the DNA sequences encoding BspQI, SNAP-Tag® and 6×His-tag. One of the constructs possesses an extra DNA sequence encoding for repeats of glycine-serine (GS) amino sequence between BspQI and SNAP-Tag® to produce a fusion protein, 6×His-SNAP-GSx6-BspQI. These plasmids were constructed following the instructions of NEBuilder® (New England Biolabs, Inc.). pHS-BspQI was made using primer pair 5′-CTT TAA GAA GGA GAT ATA CCA TGG GCA GCA GCC ACC AC-3′ (SEQ ID NO:4) and 5′-TTT TTT GCT AAT CGT CTC ATT CCT GGC GCG CCT ATA GC-3′ (SEQ ID NO:5) to generate a DNA fragment encoding 6×His-SNAP from pHS-PNK (Kant) plasmid for in-frame fusion into pMC-031 possessing BspQI coding gene prepared by amplification with primers 5′-ATG AGA CGA TTA GCA AAA AAT TC-3′ (SEQ ID NO:6) and 5′-GGT ATA TCT CCT TCT TAA AGT TAA AC-3′ (SEQ ID NO:7). pHS-GSx6-BspQI was made using primer 5′-CTT TAA GAA GGA GAT ATA CCA TGG GCA GCA GCC ACC AC-3′ (SEQ ID NO:8) and 5′ TTT TTT GCT AAT CGT CTC ATG GAA

CCA GAG CCA GAA CCG-3′ (SEQ ID NO:9) to generate a DNA fragment encoding 6×His-SNAP-GSx6 from pHS-PNK (Kan^{r}) plasmid for in-frame fusion to BspQI encoding gene in pMC-031.

Example sequences of resulting plasmid constructs are SEQ ID NOS:10-11, diagrams of which are shown in FIGS. 29A-29B. These plasmids were used for protein expression in E. coli. Plasmid DNA was mixed with competent cells and the transformation was conducted by electroporation (1800V using bacteria program in 1 mm electroporation cup). 1 mL Rich media was added and the transformants were cultured at 37° C. for 1 h. Rich media plates with 30 mg/mL chloramphenicol and 20 mg/mL kanamycin were used for transformants pick-up. The Rich media with same concentration of chloramphenicol and kanamycin was used for colony culture until OD600 reached 0.7 at 37° C. Expression of the fusion construct was initiated by addition of 100 mM IPTG and the culture was incubated at 16° C. overnight. Cells were harvested by centrifugation and lysed by sonication on ice in a buffer comprised of 50 mM Tris-HCl buffer, pH 8, 10 mM imidazole, and 0.3 M NaCl. The resulting lysate was centrifuged, and the clarified crude extract was loaded onto a column packed with Ni-NTA beads (Qiagen) for protein purification. After loading, the column was extensively washed with wash buffer (50 mM Tris-HCl buffer, pH 8, 20 mM imidazole, and 0.3 M NaCl). The fusion protein was eluted with 50 mM Tris-HCl buffer, pH 8, 250 mM imidazole, and 0.3 M NaCl), dialyzed against Diluent A buffer No BSA (NEB) overnight, and stored as aliquots in microfuge tubes at −80° C. The protein concentration was determined using Bradford assay.

### Example 16: Immobilization of SNAP-Tagged BspQI Proteins and Functional Screening

SNAP-tagged BspQI fusion proteins were immobilized onto O⁶-benzylguainine (BG)-functionalized magnetic beads without (FIG. 30A and FIG. 30D) chemical modifications, with only a surface PEG₇₅₀ coating on the beads (FIGS. 30B and 30E), or with both a surface PEG₇₅₀ coating on the beads and a PEG₄ spacer between the BG ligand and bead surface (FIGS. 30C and 30F). Chemically modified beads may be prepared as described by Li et al, 2018; Fang et al, 2021.

Three types of BG)-functionalized magnetic beads, BG-beads, BG beads with PEG₇₅₀ coating, and BG beads with PEG₇₅₀ coating and PEG4 spacer (in 100 μL of a 25% (v/v) slurry) were washed five times with 250 μL buffer (1×PBS, #9808, Cell Signaling, 1 mM DTT, 300 mM NaCl). Each type of pre-washed BG beads was mixed with either 6×His-SNAP-tag-BspQI or 6×His-SNAP-tag-GSx6-BspQI fusion protein (25 μg) in 125 μL buffer (1×PBS with 100 mM NaCl) and incubated at 4° C. overnight to immobilize the fusion protein. The enzyme bead mixture was washed with the same buffer 8 times to remove unbound protein, but the amount of unbound protein was not quantified. The enzyme bead mixture was then resuspended in 125 μL of Diluent A buffer without BSA (NEB) for storage at −80° C.

### Example 17: Enzyme Activity Assay of Immobilized BspQI

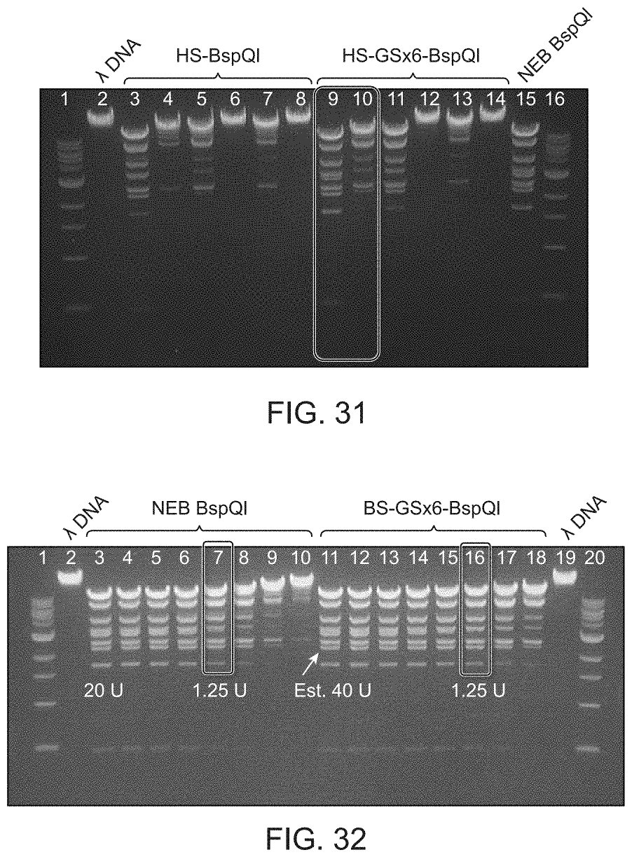

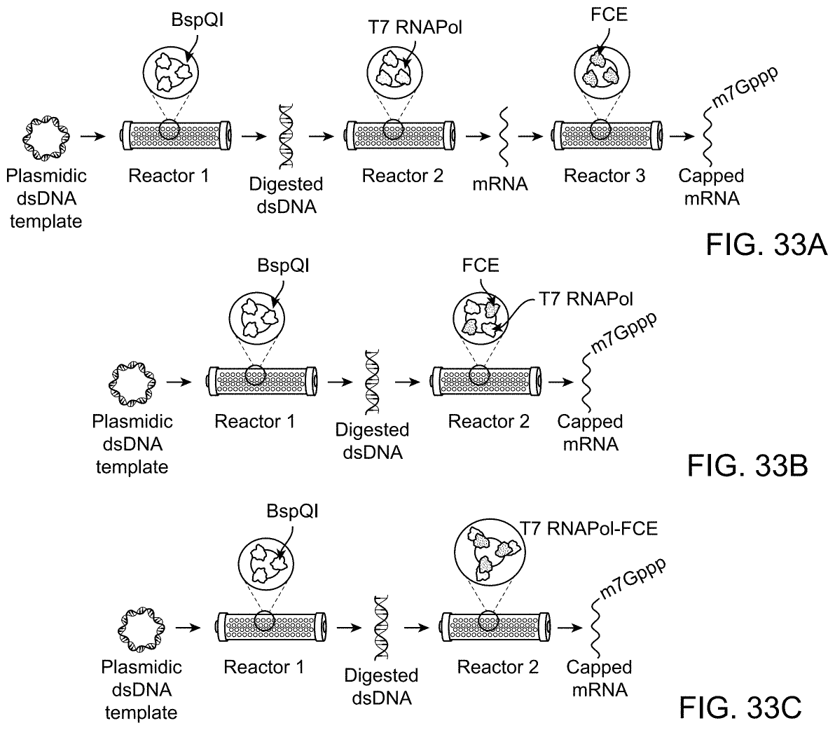

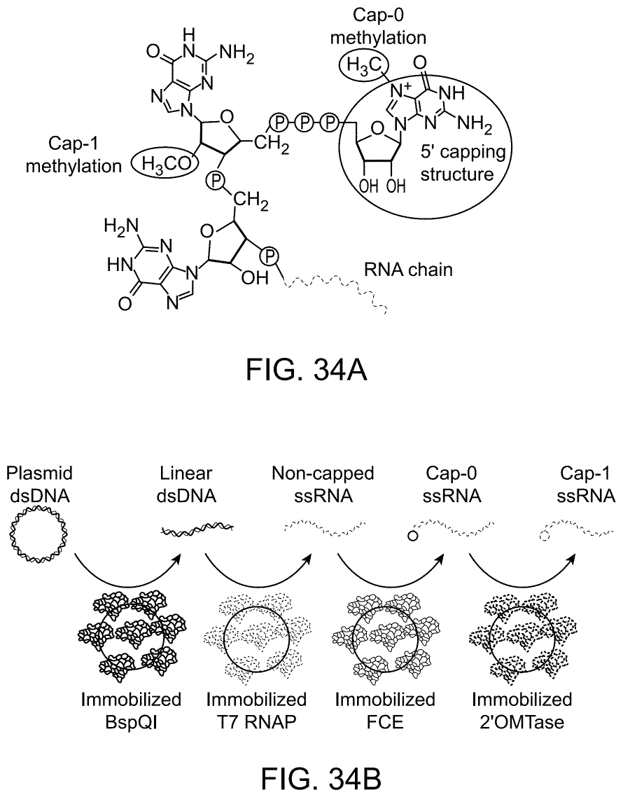

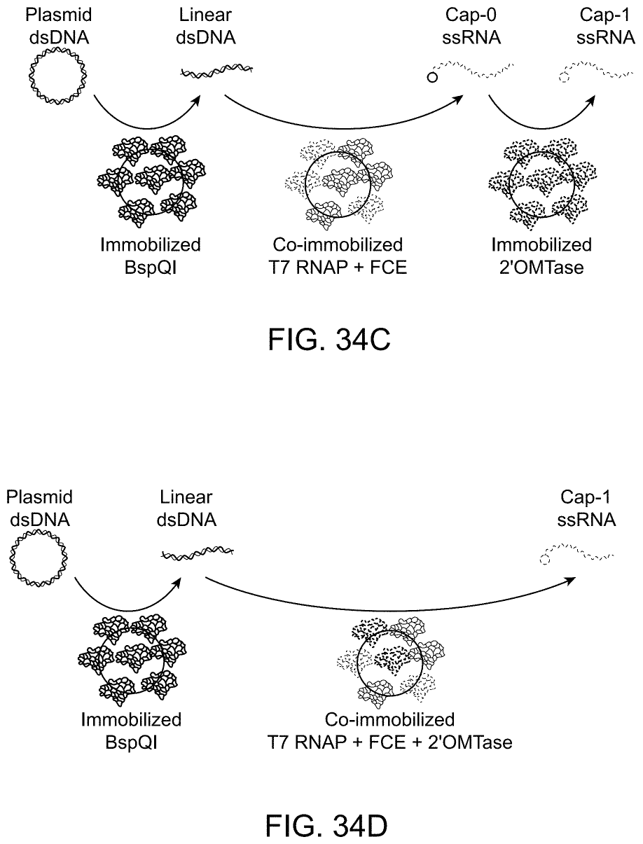

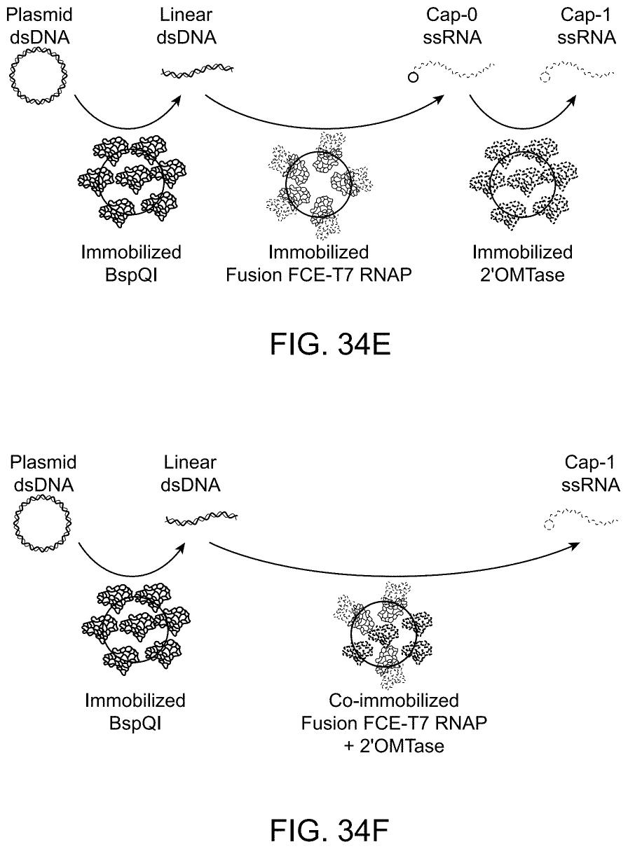

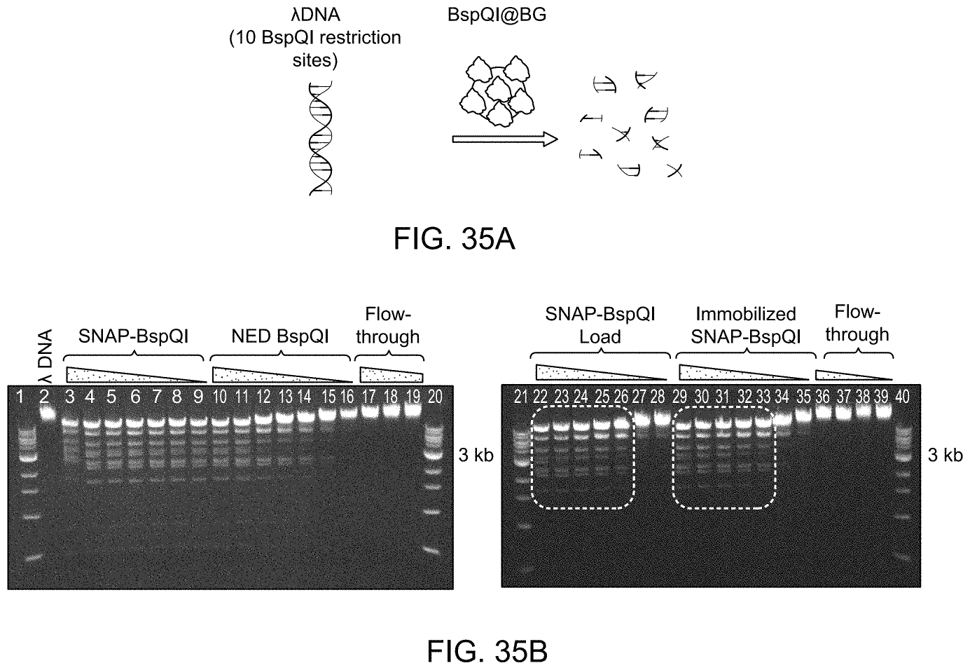

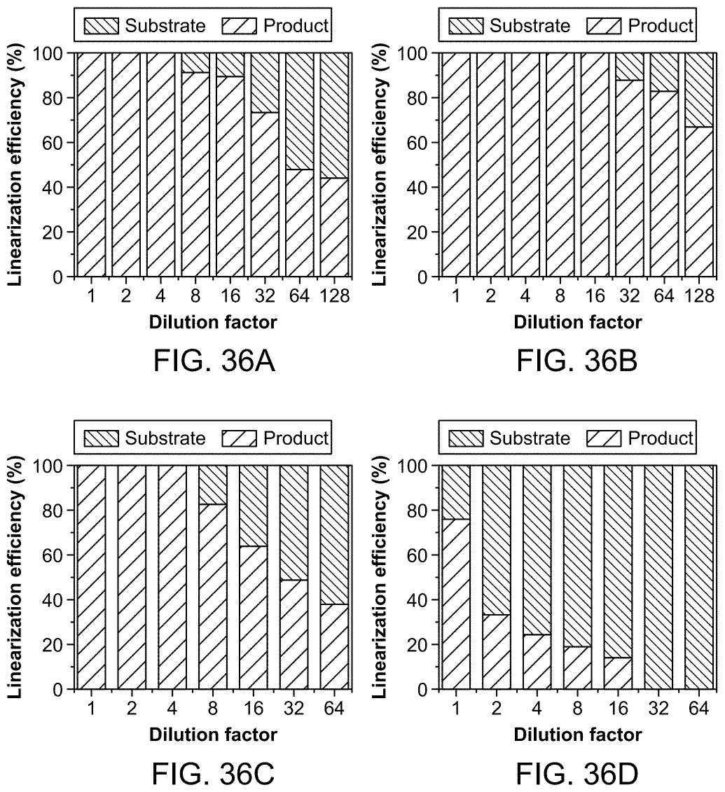

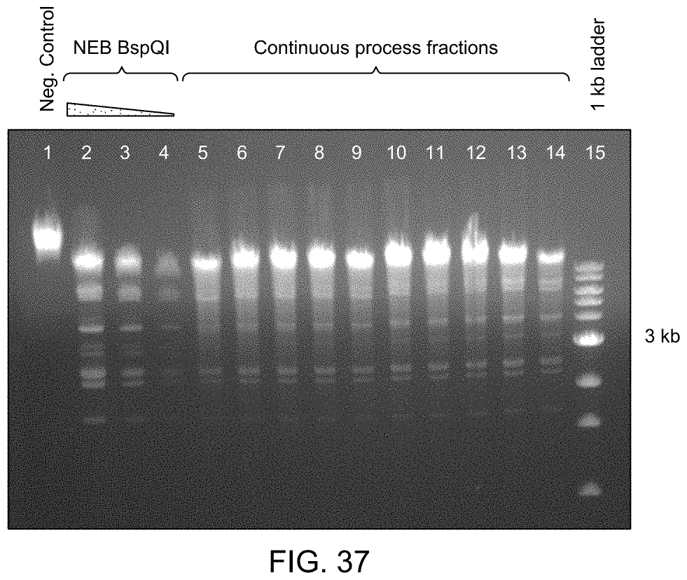

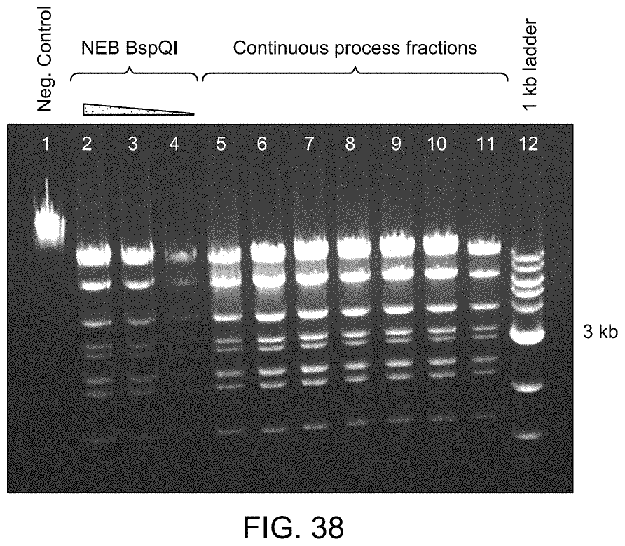

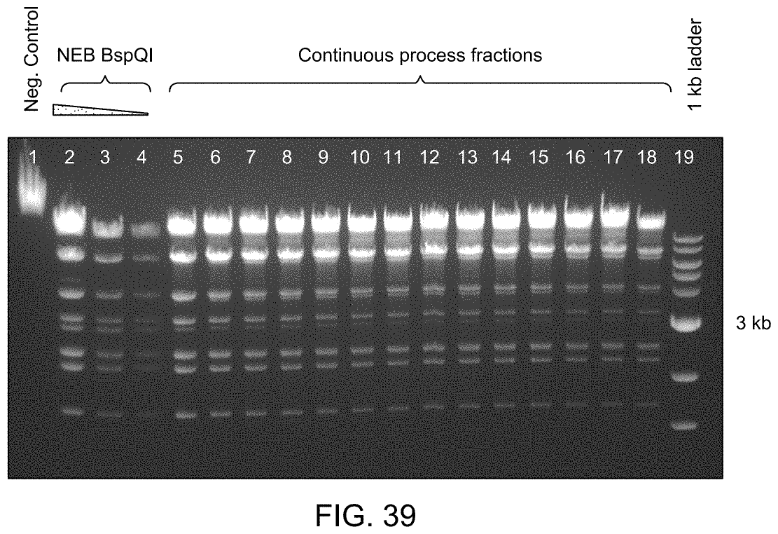

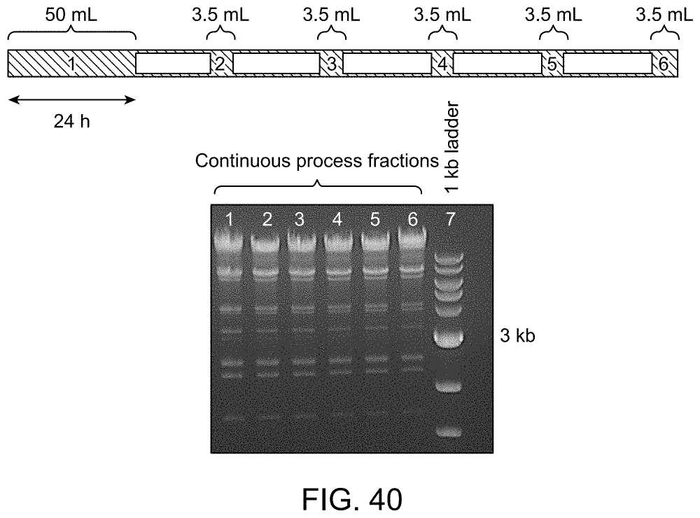

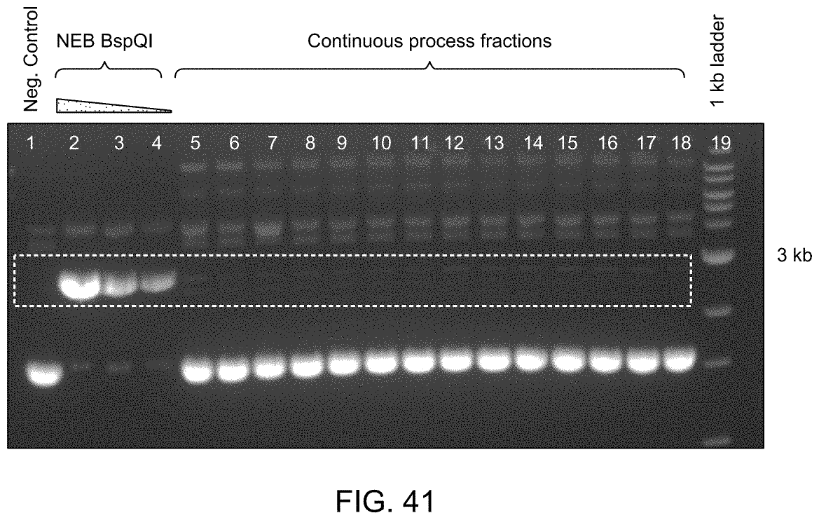

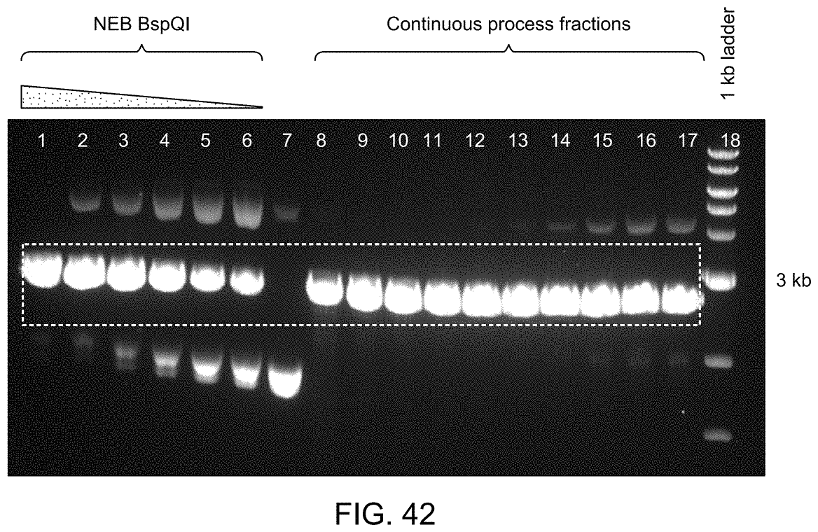

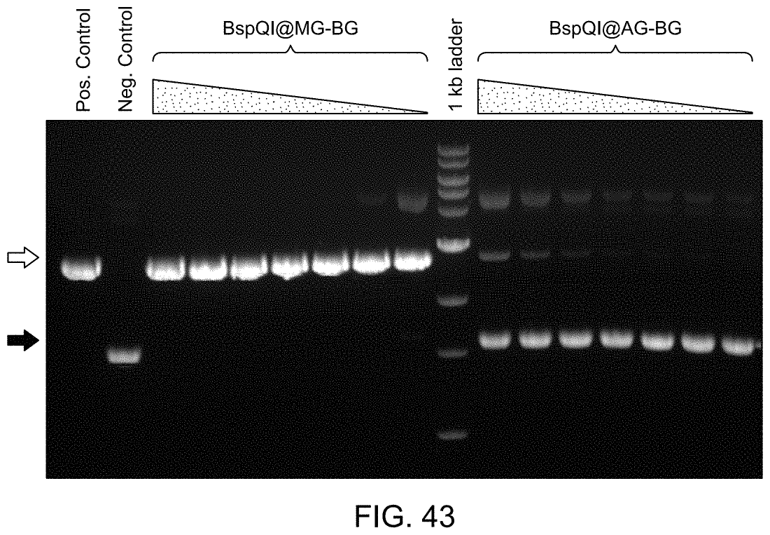

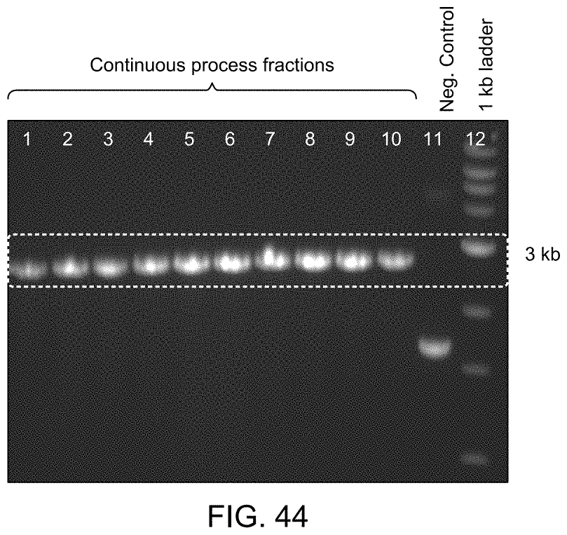

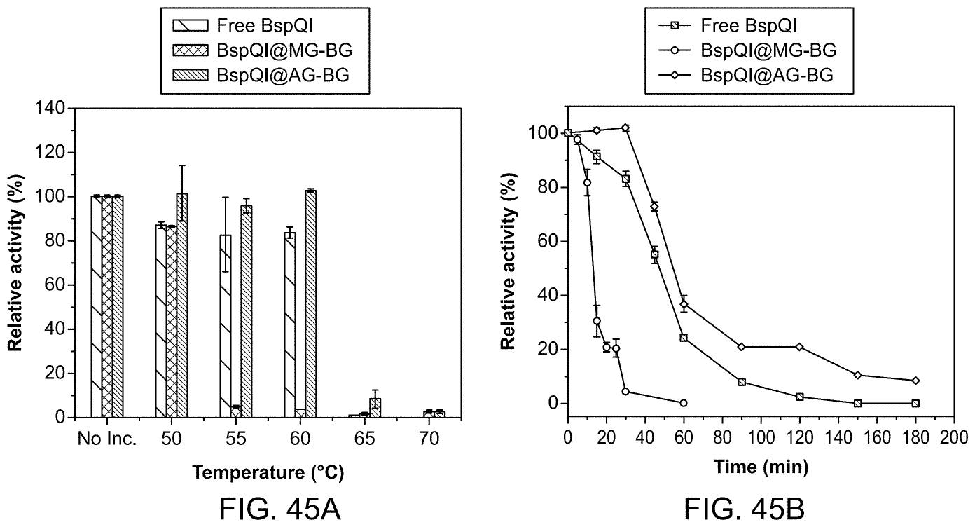

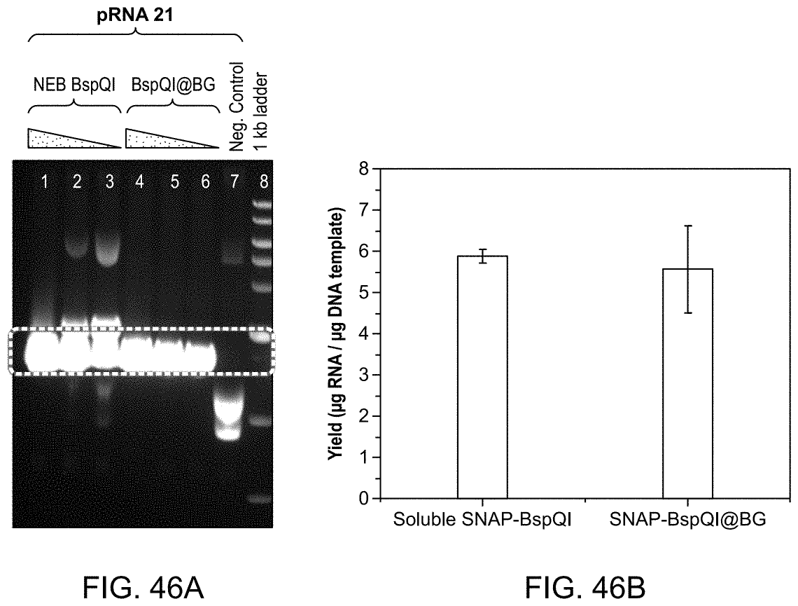

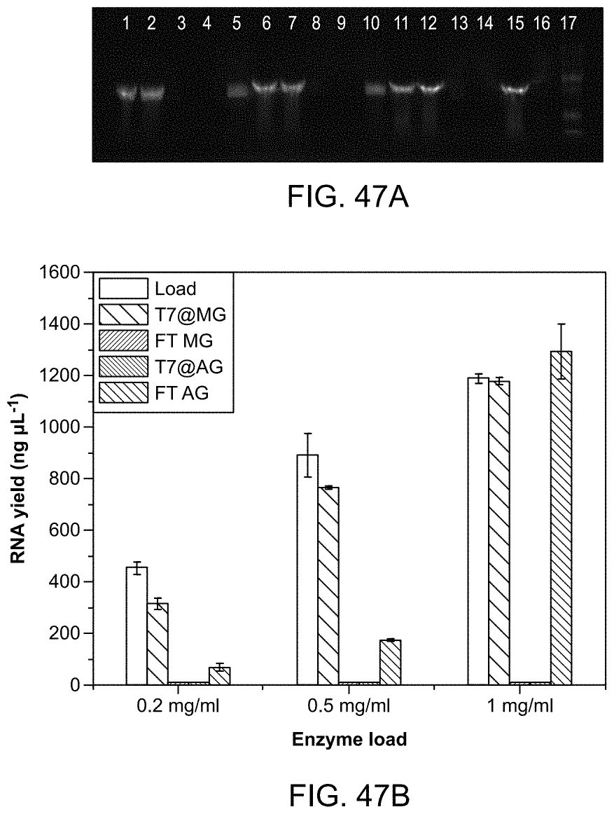

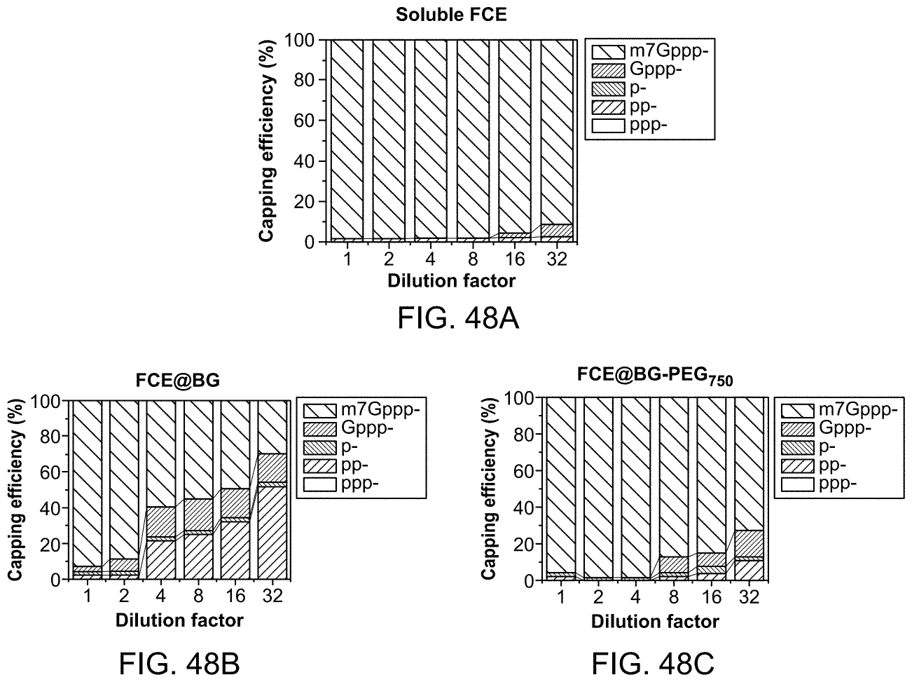

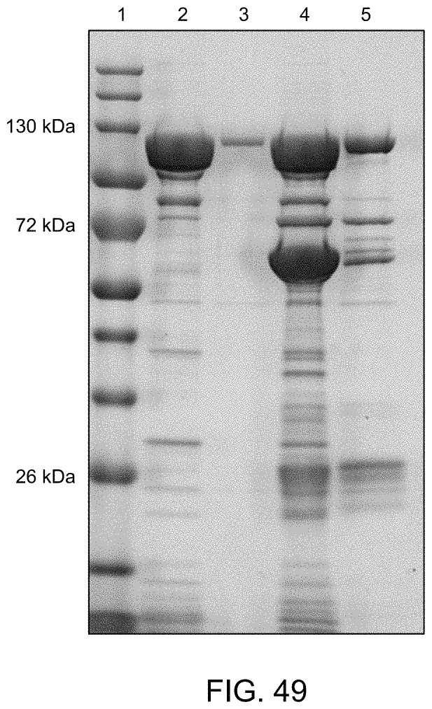

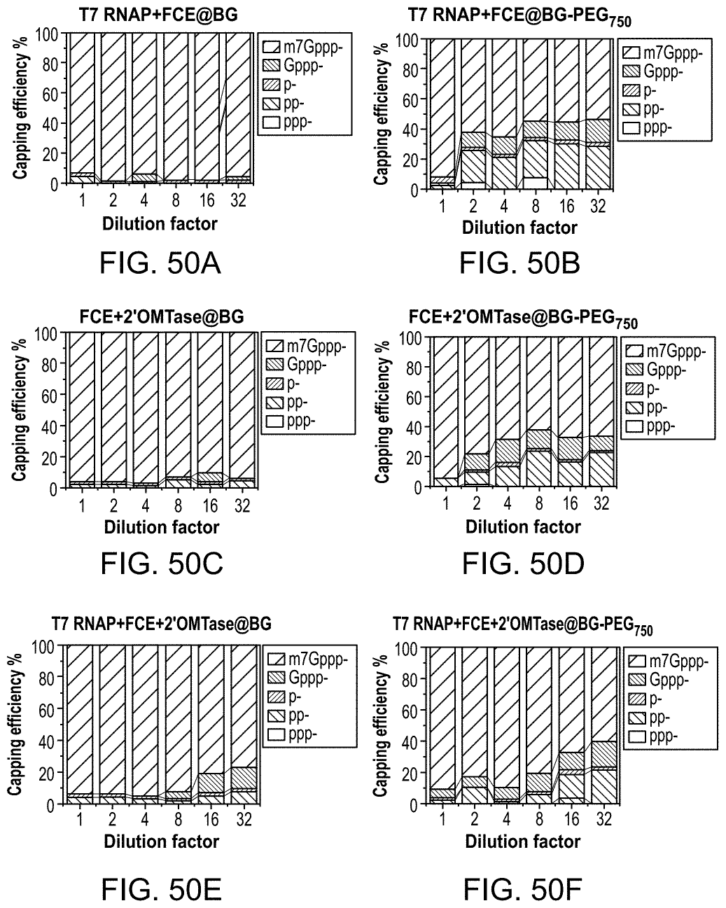

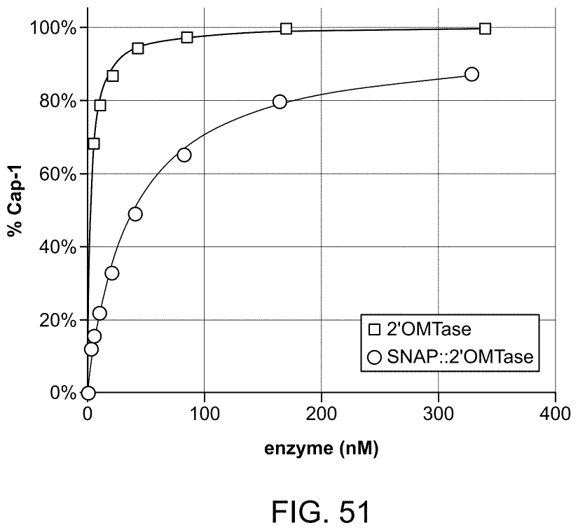

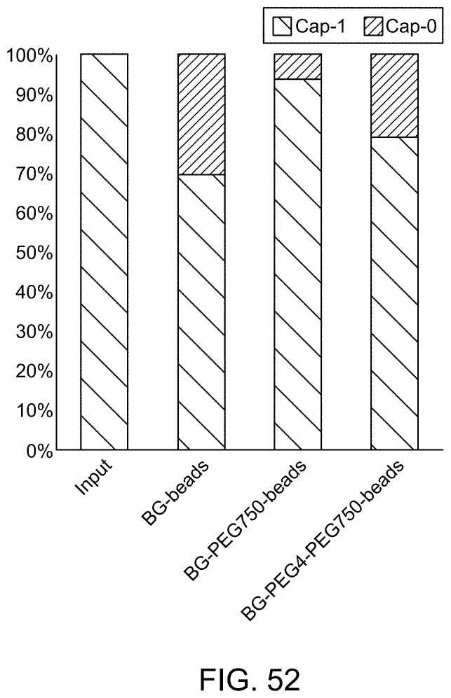

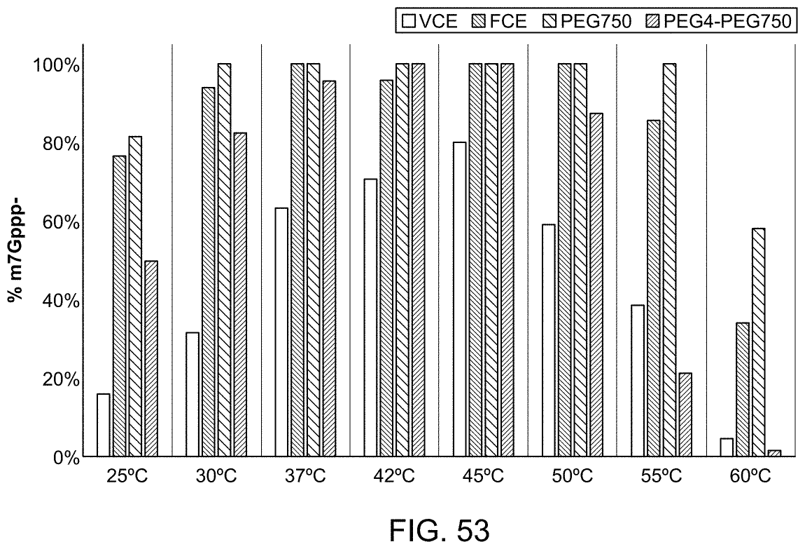

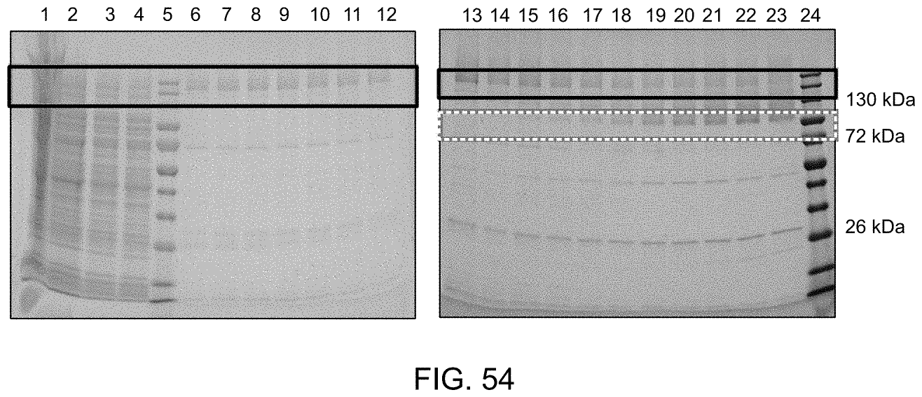

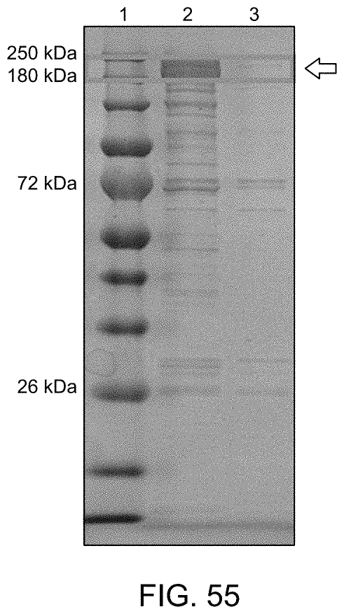

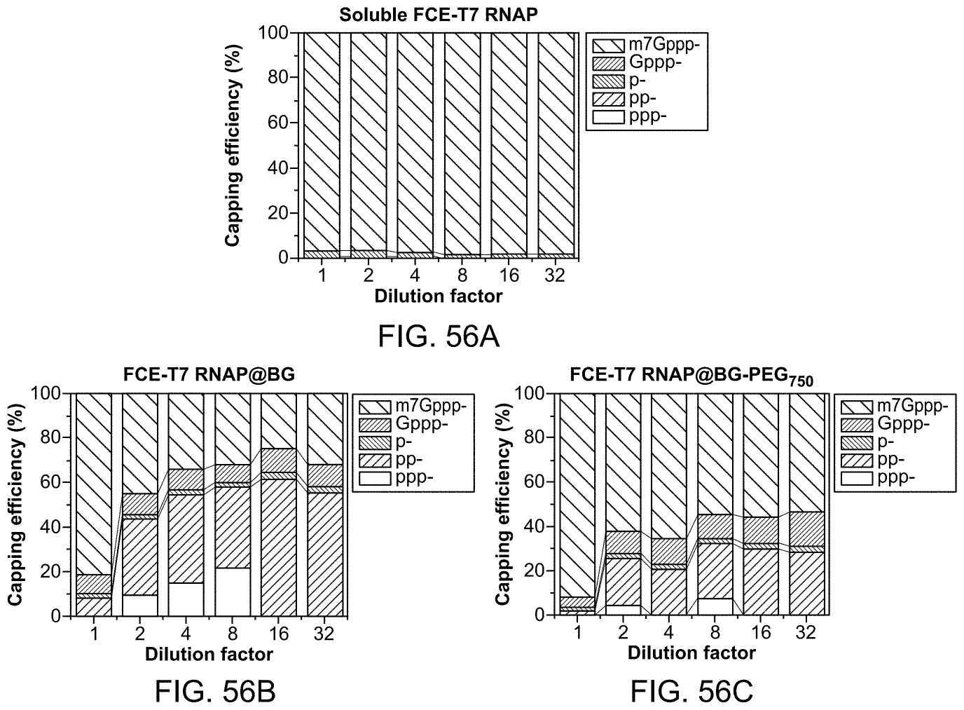

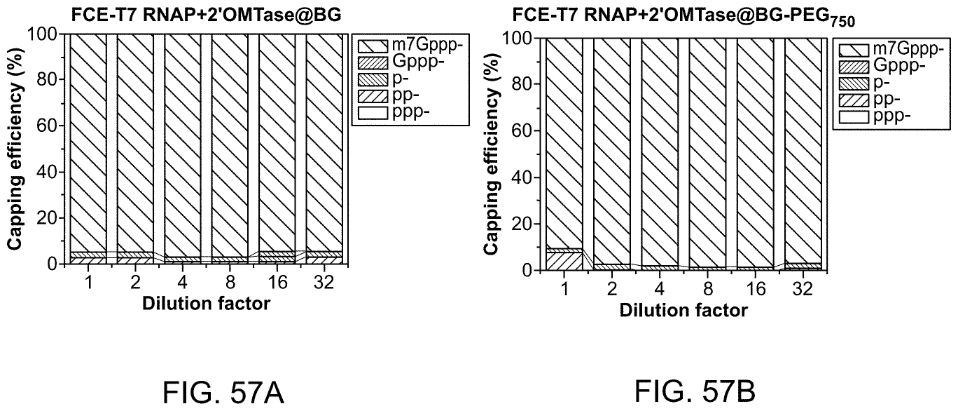

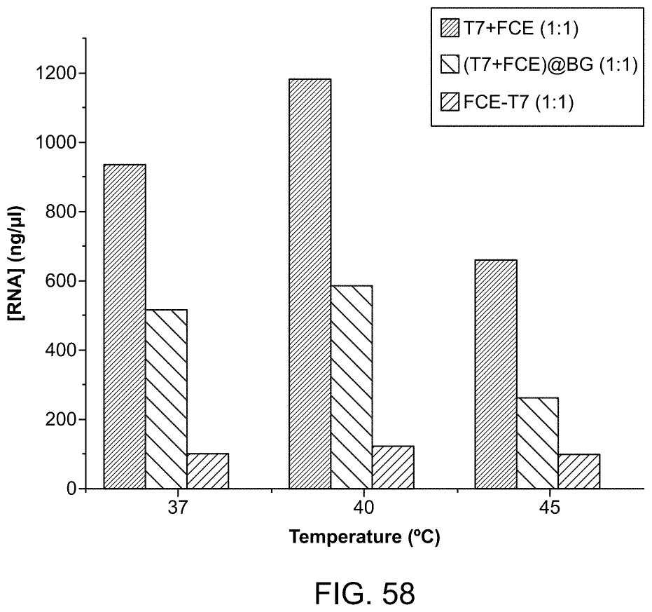

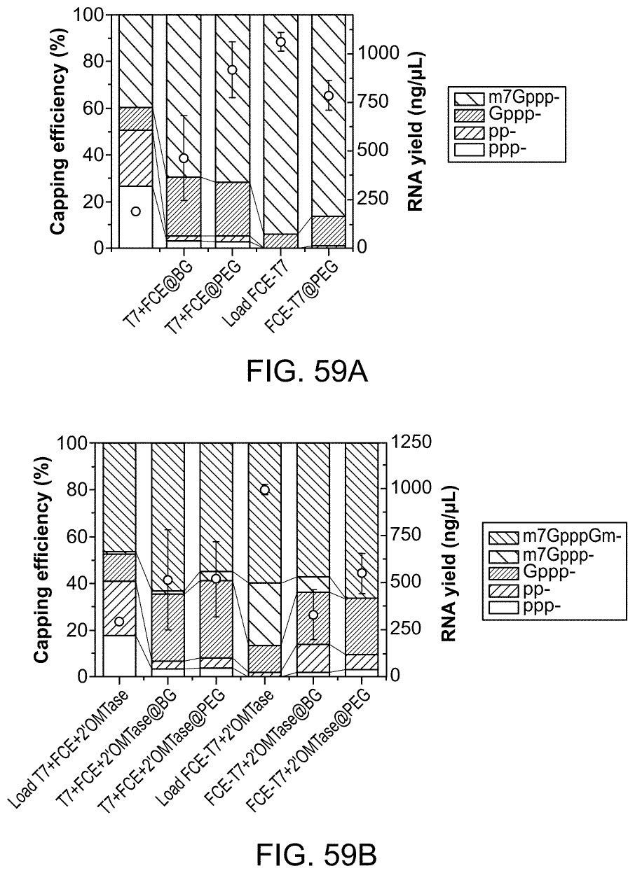

Enzyme activity of immobilized BspQI was evaluated using λ DNA as substrate. Immobilized BspQI prepared according to Examples 15 and 16 were diluted by Diluent A (no BSA) to 10 fold and 100 fold. For each enzyme assay, 15 μL nuclease-free water, 2 μL Buffer 3.1 (NEB), 2 μL λ DNA (0.5 mg/mL) and 1 μL of immobilized enzymes were mixed and incubated at 50° C. for 30 min. All immobilized enzyme preparations had an equimolar amount of BspQI. The results (shown in FIG. 31) demonstrate 6×His-SNAP-tag-GSx6-BspQI immobilized on BG modified magnetic beads without a PEG₇₅₀ coating and without a PEG₄ spacer (“HS-GSx6-BspQI”) displayed highest enzyme activity among all six enzyme-bead combinations.

### Example 18: Comparison of Enzyme Activity of Immobilized BspQI and Soluble Bspqi

Enzyme activities of example soluble and immobilized BspQI preparations were evaluated. The immobilized BspQI (HS-GSx6-BspQI, molecular weight of 72,38 Kd) sample was made using 0.2 mg protein per μL bead slurry (Example 16). Soluble BspQI (NEB #R0712L, molecular mass of 50.33 Kd) contains 0.0075 mg BspQI/mL. 2-fold serial dilutions were prepared of immobilized BspQI and soluble BspQI (NEB #R0712L) and used in activity assays as described in Example 17. Results are shown in FIG. 32. The activity of immobilized BspQI was determined as 40,000 U/mL, compared to 20,000 U/mL for soluble BspQI.

According to EXAMPLE 16, the maximum amount of fusion protein in each 1254 preparation of immobilized BspQI is 25 μg (assuming 100% retention on the magnetic beads). Accordingly, the maximum amount of fusion protein in each activity assay is 200 ng (1 μL of a 25 μg/125 μL preparation). In light of the results presented here, the specific activity of the immobilized BspQI is at least 200,000 units per milligram of enzyme. The specific activity would be even higher if less than 100% of the 25 μg of BspQI was retained on the beads. For comparison, the specific activity of soluble BspQI is estimated to be 2.7M units/mg.

### Example 19: A Schematic of In Vitro Transcription Mediated by Immobilized Enzymes

FIGS. 33 and 34 illustrate IVT schemes that take advantage of convenient removal of enzymes following an enzymatic reaction step and can be carried out in continuous flow or batch preparation. The process comprises contacting a DNA molecule (e.g., a circular DNA molecule) with a restriction enzyme (e.g., BspQI) to form a linear DNA template, contacting the linear DNA template with a DNA-dependent RNA polymerase (e.g., T7 RNA polymerase (T7 RNAP)) to produce an in vitro RNA synthesis product, (optionally) modifying the in vitro RNA synthesis product by contacting it with an RNA capping enzyme (e.g., Faustovirus Capping Enzyme (FCE)) to form a Cap-0 product, and (optionally) contacting the Cap-0 product with a 2′-O-methyltransferase (2′OMT) to form a Cap-1 product. The RNA polymerase and capping enzyme involved can be soluble or immobilized. The utility of multiple immobilized enzymes in conjunction with a single, compatible reaction buffer system may result in a highly streamlined workflow, with fewer purification steps between enzymatic treatments and, potentially, and/or less sample loss (or higher RNA yield). The enzymatic treatments may be performed consecutively or concurrently. For example, RNA synthesis enzymes (e.g., T7 RNAP) and capping enzymes (e.g., FCE) may be utilized in a one-pot formulation. For example, a one-pot formulation may use (a) separate enzymes wherein each is immobilized on its own support, (b) separate enzymes co-immobilized on a common support, or (c) a fusion protein comprising both the synthesis and capping activities, the fusion protein (optionally) immobilized on a support.

### Example 20: SNAP-BspQI Immobilization and Activity in Batch

This example demonstrates a typical immobilization reaction of SNAP-tagged BspQI enzyme using 1 μm magnetic BG-beads and subsequent characterization of the enzymatic activities of the soluble and immobilized (BspQI@MG-BG) form. The titration fractions of various samples were used to treat λDNA substrate (at a 50 μg/mL concentration) at 37° C. for 1 hour followed by agarose gel electrophoresis analysis of the digestion pattern. Results are shown in FIGS. 35A-35C 35. Both untagged BspQI (soluble) and SNAP-tagged BspQI (soluble) displayed high activity. Examination of the flow-through fractions of the immobilization reaction (lanes 17-19 and 36-39) detected little, if any, BspQI activity, indicating high conjugation efficiency. Indeed, the activity level after immobilization (lanes 21-28; left box) compared to the SNAP-BspQI Load (the soluble enzyme sample used for immobilization, lanes 29-35; right box), is consistent with high immobilization efficiency and nominal loss of activity upon immobilization.

### Example 21: Fam-Labeled Oligonucleotides as BspQI Substrate for CE Activity Assay

The enzymatic activity of various soluble and immobilized BspQI proteins was tested. NEB #R0712L, SNAP-BspQI and BspQI@MG-BG were evaluated using the following oligonucleotides as substrates:

| | | SEQ | |
|---|---|---|---|
| | Molecular | ID | |
| Protein | weight: | NO: | Substrate Sequence |
| BspQI | 18,794.3 g/mol | 13 | 5′/56FAM/TGCCGCTTT |
| Substrate | | | CTGCATCAGCACATCATC |
| FAM | | | TTCAGGCTCTTCGTCAGC |
| Forward | | | CTCGCGCCGGTTCAG-3′ |
| BspQI | 18,256.8 g/mol | 14 | 5′TGCCGCTTTCTGCATC |
| Substrate | | | AGCACATCATCTTCAGGC |
| Forward | | | TCTTCGTCAGCCTCGCGC |
| | | | CGGTTCAG-3′ |
| BspQI | 18,698.1 g/mol | 15 | 5′CTGAACCGGCGCGAGG |
| Substrate | | | CTGACGAAGAGCCTGAAG |
| Reverse | | | ATGATGTGCTGATGCAGA |
| | | | AAGCGGCA-3′ |

The FAM-labeled substrate was prepared by mixing 140 μL of nuclease-free water, 20 μL of 10× Buffer 2.1 NEB #B7202S, 20 μL of 100 μM BspQI Substrate FAM Forward and 20 μL of 100 μM BspQI Substrate Reverse. The no-FAM-labeled substrate was prepared by mixing 700 μL of nuclease-free water, 100 μL of 10×Buffer 2.1 NEB #B7202S, 100 μL of 100 μM BspQI Substrate Forward and 100 μL of 100 μM BspQI Substrate Reverse. Both mixtures were incubated for 2 minutes in a heat block set on at 85° C. and for 16 hours in the heat block turn off. After the incubation, the samples were equilibrated in cold block on ice for 1 hour. 10% FAM substrate was prepared by mixing 100 μL of 10 μM BspQI Substrate FAM Forward mix with 900 μL 10 μM BspQI Substrate no-FAM Forward mix.

Enzyme activities of soluble and immobilized BspQI preparations were evaluated. BspQI@MG-BG (SNAP-BspQI, molecular weight of 72.38 kDa) sample was made using 0.2 mg protein per mL bead slurry. Soluble SNAP-BspQI contains 0.2 mg protein/mL. Soluble BspQI (NEB #R0712L, molecular weight 50.33 kDa) contains 0.0075 mg protein/mL. 2-fold serial dilutions were prepared of immobilized and soluble SNAP-BspQI and of soluble BspQI NEB #R0712L. For each reaction, the following components were mixed: 20 μL of 10× Buffer 3.1 NEB #B7203S, 14 μL of 10% FAM substrate, 158 μL of nuclease-free water and 8 μL of each BspQI dilution. Reactions for soluble SNAP-BspQI were also tested using T7 RNAP NEB buffer #B9012S. Reaction samples were incubated 1 hour at 50° C. After incubation, EDTA was added to a final concentration of 10 mM to stop the reaction. Samples were analyzed through capillary electrophoresis.

BspQI NEB #R0712L estimated activity is 10,000 U/mL. Based on this number and the results showed in FIGS. 36A-36D, the estimated activity for BspQI NEB #R0712L is 1.33×10⁶ U/mg, and the estimated activity for both soluble and immobilized SNAP-BspQI is 1.6×10⁶ U/mL. The conclusion is that neither the SNAP-fusion insert nor the immobilization in BG beads seem to have a significant negative effect on BspQI activity. Moreover, SNAP-BspQI seems to work even better in T7 RNAP NEB buffer #B9012S than in 3.1 NEB buffer #B7203S.

### Example 22: Characterization of Immobilized BspQI Reactor in Continuous Flow

A first experiment evaluated BspQI@MG-BG in 1.5 mL suspension at 37° C. using a 5 mL of reaction mixture containing λDNA as substrate. For each enzyme assay, λDNA was diluted to 0.05 mg/mL in reaction mixture. BspQI immobilized enzyme (suspension in 10 mM Tris-HCl pH 7.4, 0.1 mM EDTA, 50 mM KCl, 1 mM DTT and 50% glycerol) was packed in a Omnifit glass column with 6.6 mm of diameter and 15 cm long. The column was introduced in a glass manifold with a temperature sensor and coupled to the reactor module of an R-series Vapourtec flow chemistry reactor. An external magnet was applied to the column to retain magnetic beads in the internal surface of the column. 1.5 mL of BspQI@MG-BG suspension was loaded in the glass column. 5 mL of reaction mixture was prepared by mixing 150 μL of 10× Buffer 3.1 NEB #B7203S, 150 μL of λDNA (0.5 mg/mL) and 1.2 mL of nuclease-free water. The flow rate in the reactor was set to 100 μL/min, and the temperature of the column was set to 37° C. 10 fractions were collected with the same time lapse (5 min) along the reaction. The data demonstrate an activity level (in the range of 5000 U/mL) for all the continuous flow fractions, with a digestion pattern comparable to that of soluble form (NEB BspQI). Results are shown in FIG. 37.

In a second experiment, 3 mL of BspQI@MG-BG suspension was loaded in the Omnifit glass column for continuous flow of 14 mL of reaction mixture. The 14 mL of reaction mixture was prepared by mixing 1.4 mL of 10×Buffer 3.1 NEB #B7203S, 1.4 mL of λDNA (0.5 mg/mL) and 11.2 mL of nuclease-free water. The flow rate in the reactor was set to 20 μL/min, and the temperature of the column was set to 37° C. 7 fractions were collected with the same time lapse along the reaction. Results shown demonstrate a higher activity than the one observed in λDNA Experiment 1 (superior to 10000 U/mL) for all the continuous flow fractions. This can be explained by the increase of the residence time of the reaction mix into the column. Results are shown in FIG. 38.

In a third experiment, a reactor packed with 3 mL of BspQI@MG-BG suspension in the Omnifit glass column was evaluated in continuous flow of a 50 mL of reaction mixture at 37° C. The 50 mL reaction mixture was prepared by mixing 5 mL of 10×Buffer 3.1 NEB #B7203S, 5 mL of λDNA (0.5 mg/mL) and 40 mL of nuclease-free water. The flow rate in the reactor was set to 50 μL/min, and the temperature of the column was set to 37° C. 14 fractions were collected with the same time lapse along the reaction. Results shown demonstrate a higher activity than the one observed in λDNA Experiment 1 (superior to 10000 U/mL) for all the continuous flow fractions. This experiment demonstrates the successful operation of the catalyst for a larger substrate volume scale. Results are shown in FIG. 39.

In a fourth experiment, λDNA by was digested by BspQI@MG-BG during 24 hours of a continuous flow run. 50 mL of λDNA substrate at a 50 μg/mL concentration was passed through a 3 mL column heated to 37° C. for 16.7 hours (at a flow rate of 50 μL/min; Fraction 1). Five additional 3.5 mL of λDNA-containing fractions were pumped through the 3 ml column and collected at the interval time indicated (Fractions 2-6). Agarose gel electrophoresis analysis indicate efficient digestion of λDNA in all the flow-through fractions. Results are shown in FIG. 40.

### Example 23: Examination of BspQI Leaching from a BspQI@MG-BG Packed-Bed Reactor

An example reactor packed with magnetic beads immobilized with BspQI was tested in continuous flow for possible leaching. BspQI activity present in the flow-through fractions of λDNA digestion by BspQI@MG-BG was analyzed by linearization of pUC19 plasmid DNA possessing a single BspQI recognition sequence. pUC19 plasmid DNA (at a 50 μg/mL concentration) was treated at 37° C. for 60 min with the 14 fractions collected from the continuous flow run (shown in EXAMPLE 22C). Agarose gel electrophoresis analysis reveals the absence of a dominant DNA species corresponding to linear pUC19 DNA (FIG. 41, box; compare lanes 2-4 positive control vs lanes 5-18), indicating little or very low leaching from the BspQI column during the passage of pUC19 substrate.

### Example 24: pUC19 Digestion in BspQI@MG-BG Packed-Bed Reactor

This example shows digestion of plasmid DNA pUC19 by an immobilized BspQI reactor in continuous flow. 3 mL of BspQI@MG-BG suspension was loaded in the glass column. 50 mL of reaction mixture was prepared by mixing 5 mL of 10× Buffer 3.1 NEB #B7203S, 2.5 mL of pUC19 (1 mg/mL) and 42.5 mL of nuclease-free water. The flow rate in the reactor was set to 30 μL/min, and the temperature of the column was set to 50° C. 10 fractions were collected with the same time lapse along the reaction. Results are shown in FIG. 42. The data indicate efficient digestion of pUC19 plasmid DNA to generate a linear DNA product (indicated in box, FIG. 42) albeit a small amount of undigested pUC19 DNA was observed in the final flow-through fractions as tested (Lane 14-17).

### Example 25: Comparison of BspQI Immobilized onto Magnetic or Agarose Beads

This example compares the retained activity when 30 μg of SNAP-BspQI were loaded to either 33 mg of agarose beads (BspQI@AG-BG) or 1 mg of sera magnetic beads (BspQI@MG-BG). The titration fractions of various preparations were used to treat pUC19 substrate (at a 50 μg/mL concentration) at 50° C. for 1 hour followed by agarose gel electrophoresis analysis of the reaction products. Results are shown in FIG. 43. BspQI@MG-BG dilutions (from 1:1 to 1:64) shown in lanes 3-9 displayed complete linearization of pUC19 (hollow arrow). BspQI@AG-BG dilutions (from 1:1 to 1:64) shown in lanes 11-17 displayed partial digestion of pUC19 (bands around both the hollow and solid arrows) and a lower activity corresponded to an increased dilution factor of BspQI@AG-BG (from 1:1 to 1:64).

### Example 26: pUC19 Digestion in BspQI@AG-BG Packed-Bed Reactor

This example demonstrates digestion of circular plasmid DNA pUC19 by a reactor packed with immobilized BspQI beads in continuous flow. 2 mL column volume (3.28 g) of BspQI@AG-BG was loaded in the glass column. 50 mL of reaction mixture was prepared by mixing 5 mL of 10×Buffer 3.1 NEB #B7203S, 2.5 mL of pUC19 (1 mg/mL) and 42.5 mL of nuclease-free water. The flow rate in the reactor was set to 30 μL/min, and the temperature of the column was set to 50° C. 10 fractions were collected with the same time lapse along the reaction. Results are shown in FIG. 44. The data indicate efficient digestion of pUC19 plasmid DNA to generate a linear DNA product (indicated in box, lane 1-10).

### Example 27: BspQI Thermostabiliity

This example shows analysis of thermostability of immobilized BspQI using CE assay. An aliquot of soluble SNAP-BspQI, BspQI@MG-BG and BspQI@AG-BG was incubated for 30 min at different temperatures (50, 55, 60, 65 and 70° C.). CE assay described in Example 21 was performed before and after the incubation, and the activity of the enzyme after each incubation was represented as relative activity compared to the non-incubated reaction. As observed in the figure below, SNAP-BspQI and BspQI@AG-BG displayed more than 80% of their initial activity until 60° C., and their activity dropped at 65° C. It is remarkable that BspQI@AG-BG showed a relative activity slightly higher than SNAP-BspQI. On the contrary, BspQI@MG-BG presented a lower thermostability, loosing almost all its initial activity when incubated at 55° C. With these results in hand, SNAP-BspQI, BspQI@MG-BG and BspQI@AG-BG were incubated at 60° C., and the CE assay described in Example 21 was performed after different incubation times: 0, 5, 10, 15, 20, 25, 30 and 60 for BspQI@MG-BG, and 0, 15, 30, 45, 60, 90, 120, 150 and 180 for SNAP-BspQI and BspQI@AG-BG. Relative activity is again represented comparing the product after each time of incubation with the product obtained in the reaction when the enzyme was not incubated. Results are shown in FIGS. 45A-45B and illustrate that BspQI@AG-BG presents a higher thermostability than soluble SNAP-BspQI for longer term incubations, whereas BspQI@MG-BG activity drops more quickly.

### Example 28: Generation of DNA Template by Immobilized Bspqi for In Vitro Transcription

This example demonstrates that immobilized BspQI generated linear DNA template for in vitro transcription by T7 RNA polymerase without a purification step. pRNA21 possessing two recognition sequences of BspQI was treated with a BspQI enzyme to generate a DNA template for synthesis of run-off transcripts possessing a 3′ polyA tail. Specifically, pRNA21 at 50 μg/mL concentration was digested by either soluble or immobilized BspQI at 50° C. for 1 hour. The samples were subjected to agarose gel electrophoresis analysis, to examine digestion efficiency (as shown in FIG. 46A with linearized DNA highlighted by the box). Following the treatment, immobilized BspQI was pelleted by magnet and the reaction medium, with a 100% DNA recovery yield, was transferred to a fresh Eppendorf tube for RNA synthesis with T7 RNA polymerase (#NEB M0251S), while the DNA template in the soluble enzyme reaction was purified with a recovery yield of approximately 70%. In vitro transcription reaction was performed using the same amount of each recovered DNA template by mixing the following components: 9.5 μL nuclease-free water, 2 μL 10× Reaction Buffer (#NEB B9012S), 4 μL rNTP mix (25 mM, #NEB N04665), 0.5 μL digested pRNA21 DNA template (500 ng), 2 μL MgCl₂ (140 mM), 0.5 μL yeast inorganic pyrophosphatase (100 U/mL, #NEB M2403S), 0.5 μL murine RNase inhibitor (40,000 U/mL, #NEB M0314S) and 2 μL of T7 RNA polymerase. After the reaction, 1 μL Turbo DNase I (2 U/μL, Invitrogen) was added to each sample, which were incubated 30 min more at 37° C. RNA was purified by using #NEB T2050L Monarch kit. The RNA yields of the transcript (173 nucleotides in length) were measured by Nanodrop (FIG. 46B), indicating that both protocols result in essentially the same RNA yields when the same amount of the pRNA21 DNA template recovered after the template preparation was utilized for each IVT reaction.

### Example 29: T7 RNA Polymerase Immobilization for CLUC DNA IVT

This example shows SNAP-T7 RNA polymerase activity when immobilized to sera magnetic and agarose benzyl guanine functionalized beads, respectively, at different protein loads. Beads were washed 5 times with one volume of 1×phosphate buffer saline and 1 mM dithiothreitol (DTT). 150 μL of enzyme suspension (in the same buffer) at 0.2; 0.5 and 1 mg/mL were loaded into whether 1 mg of sera magnetic beads (T7@MG-BG) or 33 mg of agarose beads (T7@AG-BG), which were incubated at 4° C. overnight. Flow-through was recovered and beads were washed 5 times more with the same buffer, and finally resuspended in 100 μL of 50 mM Tris-HCl (pH 8) 100 mM NaCl, 20 mM DTT, 1 mM EDTA, 50% glycerol, 0.1% Triton X-100.

cLuc DNA template was previously digested by mixing the following components: 670 μL nuclease-free water, 229 μL cLuc plasmid DNA (87.5 ng/μL), 100 μL rCutSmart buffer (#NEB B6004S), 1 μL NotI (10 U/mL, #NEB R0189S). The mixture was incubated for 1 h at 37° C. and cleaned with #NEB T1030L Monarch kit. DNA concentration was determined through Nanodrop. In vitro transcription reactions were performed as described in Example 27, but using T7 RNA polymerase load, immobilized suspension or flow-through. Results are shown in FIGS. 47A and 47B.

As observed in FIG. 46B, the recovered activity of T7@MG-BG when compared to the load is superior to 80% for all the enzyme loads (achieving 100% at 1 mg/mL enzyme load), and no activity was observed in the flow-through fractions.

### Example 30: Capping Assay for Soluble and Immobilized Faustovirus Capping Enzyme

This example describes Faustovirus capping enzyme (FCE) capping efficiency in its soluble and immobilized form. 0.05 mg of FCE was immobilized in 100 μl of BG-coated sera magnetic beads (with and without PEG750 coating) by incubating with 1×PBS, 1 mM DTT overnight at 4° C. After washing the beads, they were resuspended in 100 μl of FCE storage buffer (20 mM Tris-HCl pH 8, 100 mM NaCl, 1 mM DTT, 20 mM L-Arg, 50% Glycerol). Enzyme activity was screened using a FAM-labeled RNA oligonucleotide as substrate. For every sample, the following components were assembled: 6.5 μl nuclease-free H₂O, 1 μl 10×capping buffer (50 mM Tris-HCl pH 8, 5 mM KCl, 1 mM MgCl₂, 1 mM DTT), 0.5 μl SAM (2 mM), 0.5 μl GTP (10 mM), 0.5 μl ppp25mer[FAM] substrate (1 μM) and 1 μl FCE (starting at 0.05 mg/ml). Reactions were also performed with serial 1:2 dilutions of the concentrated enzyme. Reactions were incubated at 37° C. for 30 minutes and quenched by adding 10 μl of stop reagent (20 mM EDTA, 2% SDS). Reactions were analyzed through capillary electrophoresis. As shown in FIGS. 48A-48C, capping efficiency is more than 95% for soluble FCE in all the dilutions. Immobilized enzymes effectively capped mRNA with a capping efficiency of 80% for enzyme immobilized on PEG750 coated beads until 1:32 dilution and 60% for enzyme immobilized on non-PEG750 coated beads from 1:4 dilution.

### Example 31: Capping Assay for Co-Immobilized T7 RNA Polymerase, Faustovirus Capping Enzyme, and 2′O-Methytransferase

FCE capping efficiency was assessed in co-immobilization samples of three different combinations: SNAP-T7 RNAP+SNAP-FCE; SNAP-FCE+SNAP-2′OMTase; SNAP-T7 RNAP+SNAP-FCE+SNAP-2′OMTase. In each case, 0.1 mg of SNAP-T7 RNAP, 0.05 mg of SNAP-FCE and/or 0.05 mg of SNAP-2′OMTase were immobilized in 100 μl of BG-coated sera magnetic beads (with and without PEG750 coating) as described in Example 30. Results are shown in FIG. 49 for PEG750 beads. Capping efficiency of each sample was determined through the CE assay also described in Example 30. As shown in FIGS. 50A to 50F, co-immobilization (both of SNAP-T7 RNAP with SNAP-FCE and SNAP-FCE with 2′OMTase) in non-PEG750 coated beads displayed a capping efficiency higher than 80% for all the tested dilutions. Under conditions tested, PEG750 coated beads displayed a capping efficiency of 60% in the T7 RNAP+FCE@BG-PEG750 1:2 dilution, in the FCE+2′OMTase@BG-PEG750 1:8 dilution and in the T7 RNAP+FCE+2′OMTase@BG-PEG750 1:32 dilution.

### Example 32: Capping Assay for Soluble and Immobilized Vaccinia Cap 2′O-Methytransferase

Capping efficiency vaccinia cap 2′-O-methyltransferase was assessed in its soluble form with and without the SNAP-tag and in its SNAP-tagged immobilized form. 0.05 mg of vaccinia cap 2′-O-methyltransferase were immobilized in 100 μl of BG-coated sera magnetic beads (with and without PEG750 coating) by incubating with 1×PBS, 1 mM DTT overnight at 4° C. After washing the beads, they were resuspended in 100 μl of FCE storage buffer (20 mM Tris-HCl pH 8, 100 mM NaCl, 1 mM DTT, 20 mM L-Arg, 50% Glycerol). Enzyme activity was screened using a 25 μL synthetic RNA oligonucleotide with a 5′ Cap-0 structure (SEQ ID NO: 20). For every sample, the following components were assembled: 6.5 μl nuclease-free H₂O, 1 μl 10× capping buffer (500 mM Tris-HCl pH 8, 50 mM KCl, 10 mM MgCl₂, 10 mM DTT), 0.5 μl SAM (2 mM), 0.5 μl of the 25mer substrate with a 5′ Cap-0 structure (10 μM) and 1 μl of vaccinia cap 2′-O-methyltransferase at varying protein concentrations. Reactions were incubated at 37° C. for 30 minutes and quenched by adding 2 μl of 30 mM EDTA and heating at 75° C. for 5 min. The extent of methylation was then assessed using intact LC-MS analysis. As shown in the figure below, the specific activity (quantity of Cap-1 produced per enzyme concentration) of the SNAP-2′OMTase fusion is lower than its counterpart without the SNAP-tag. Results are shown in FIG. 51.

After quenching using EDTA and heat, the extent of methylation was assessed using intact LC-MS analysis. As shown in FIG. 52, the immobilized SNAP-2′OMTase maintained 70%-90% enzyme activity compared to the immobilization input. SNAP-2′OMTase immobilization on BG-PEG750 beads exhibits highest enzyme activity as tested.

### Example 33: Operational Temperature Range of Immobilized SNAP-FCE

Enzymatic activity of immobilized SNAP-FCE was assessed over a wide range of reaction temperature. SNAP-FCE was immobilized on BG-coated sera magnetic beads (with and without PEG750 or PEG4-PEG750 coating) as described in EXAMPLE 30. Enzyme activity was assayed using a 25 μL synthetic RNA oligonucleotide with 5′ triphosphate. For every sample, the following components were assembled: 6.5 μl nuclease-free H₂O, 1 μl 10× capping buffer (500 mM Tris-HCl pH 8, 50 mM KCl, 10 mM MgCl₂, 10 mM DTT), 0.5 μl SAM (2 mM), 0.5 μl of the 25mer substrate with 5′ triphosphate and 3′ FAM group (10 μM), and 1 μl of the immobilized SNAP-FCE, soluble FCE or VCE at 10 nM protein concentrations. Reactions were incubated at 25 to 60° C. for 30 minutes. After quenching with 10 μl of 2×Quench Solution (20 mM EDTA, 2% SDS), the extent of RNA capping was assessed using capillary electrophoresis. As shown in the figure below, SNAP-FCE immobilized on BG-PEG750 beads generated equal or more Cap-0 structure (m7Gppp-) at temperatures spanning 25-60° C. Results are shown in FIG. 53.

### Example 34: Expression and Purification of Fusion SNAP-FCE-T7 RNAP

A SNAP-FCE-T7 RNAP fusion construct was prepared and assayed for one-pot co-transcriptional capping. The construct possesses a DNA sequence encoding SNAP-tag®, FCE and T7 RNAP, and was obtained following the instructions of NEBuilder (New England Biolabs, Inc). A SNAP-FCE-T7 RNAP was made using primer pair:

(SEQ ID NO: 13)
5′- atcatcatggcgcgggcaccaaaatcgaagaagacaaggactgcga
aatgaaacgtaccaccctggatagcccg-3′
and
(SEQ ID NO: 14)
5′- caacgctgcagacgcttcgcgcccgcggtcgcgccgcc-3′

to generate a DNA fragment encoding SNAP gene for in-frame fusion into pTac possessing both FCE and T7 RNAP genes prepared by amplification with primers:

(SEQ ID NO: 15)
5′- gcgaagcgtctgcagcgttg-3′
and
(SEQ ID NO: 16)
5′- ggtgcccgcgccatgatg-3′.

The resulting plasmid was used for protein expression in E. coli. Plasmid was mixed with competent cells and the transformation was conducted by thermal shock. 950 μl of rich media was added and the transformants were cultured at 37° C. for 1 h. Rich media plates with 20 mg/ml kanamycin were used for transformants pick-up. The Rich media with same concentration of kanamycin was used for colony culture until OD600 reached 0.6 at 37° C. Expression of the fusion construct was initiated by addition of 100 mM IPTG and the culture was incubated at 18° C. overnight. Cells were harvested by centrifugation and lysed by sonication on ice in a buffer comprising 50 mM Tris-HCl pH 8, 50 mM NaCl, 20 mM L-arginine and 2% glycerol. The resulting lysate was centrifuged, and the clarified crude extract was loaded onto a column packed with Ni-NTA beads (Qiagen) for protein purification. After loading, the column was extensively washed with buffer (20 mM Tris-HCl pH 8, 50 mM NaCl, 20 mM imidazole, 20 mM L-arginine). The fusion protein was eluted with 20 mM Tris-HCl pH 8, 150 mM NaCl, 500 mM imidazole, 20 mM L-arginine, and dialyzed overnight against 40 mM Tris-HCl pH 8, 100 mM NaCl, 50 mM L-arginine, 0.1 mM DTT, 50% glycerol. The protein presence was assessed through SDS-PAGE and its concentration was determined through Bradford assay. As shown in FIG. 54, after lane 18 the main protein band corresponds to the weight of SNAP-FCE, which could be explained by a protease partial cleavage of T7 RNAP from the fusion protein. To avoid the mixture of SNAP-FCE-T7 RNAP and SNAP-FCE, fractions 6-17 (in which the presence of SNAP-FCE is much lower) were mixed to be dialyzed as previously described.

### Example 35: Capping Assay for Soluble and Immobilized Fusion SNAP-FCE-T7 RNAP and Co-Immobilized SNAP-FCE-T7 RNAP and 2′O-Methytransferase

Capping efficiency for SNAP-FCE-T7 RNAP fusion protein in its soluble and immobilized forms were assayed. 0.1 mg of SNAP-FCE-T7 RNAP was immobilized in 100 μl of BG-coated sera magnetic beads (with and without PEG750 coating) by incubating with 1×PBS, 1 mM DTT overnight at 4° C. After washing the beads, they were resuspended in 100 μl of FCE storage buffer (20 mM Tris-HCl pH 8, 100 mM NaCl, 1 mM DTT, 20 mM L-Arg, 50% Glycerol). Capping efficiency of each sample was determined through the CE assay also described in Example 30. As shown in FIGS. 55 and 56, capping efficiency exceeded 95% for all the dilutions of the soluble SNAP-FCE-T7 RNAP. Immobilized fusion protein preparations capped RNA with an efficiency of 60% for PEG750-coated magnetic beads at 1:2 to 1:32 dilutions and an efficiency of 40% for non-PEG750 coated beads.

### Example 36: Capping Assay for Co-Immobilized SNAP-FCE-T7 RNAP and SNAP-2′O-Methytransferase

Capping efficiency was assessed for co-immobilized SNAP-FCE-T7 RNAP and SNAP-2′OMTase. In each case, 0.1 mg of FCE-T7 RNAP and 0.05 mg of 2′OMTase were immobilized in 100 μl of BG-coated sera magnetic beads (with and without PEG750 coating) as described in Example 30. Capping efficiency of each sample was determined through the CE assay also described in Example 30. Comparing FIGS. 57A-57B with FIGS. 56B-56C, it is noticeable that the presence of 2′OMTase positively affects capping efficiency of immobilized SNAP-FCE-T7 RNAP with an efficiency of 90% observed for all tested dilutions.

### Example 37: RNA Synthesis at Different Temperatures

The impact of temperature on transcription was assessed. Three different combinations were assayed: (a) a mixture of soluble T7 RNAP and soluble FCE (“T7+FCE”); (b) co-immobilized T7 RNAP and FCE (on non-PEG750 coated magnetic beads; “(T7+FCE)@BG”); and (c) soluble fusion FCE-T7 RNAP (“T7-FCE”).

In vitro co-transcriptional capping was performed by mixing the following components: 2.75 μL nuclease-free water, 1.7 μL 10× Reaction Buffer (#NEB B9012S), 4 μL rNTP mix (25 mM, #NEB N04665), 3.3 μL digested pRNA21 DNA template (500 ng), 2 μL MgCl₂ (140 mM), 0.5 μL yeast inorganic pyrophosphatase (100 U/mL, #NEB M2403S), 0.5 μL murine RNase inhibitor (40,000 U/mL, #NEB M0314S), 0.25 μL SAM (32 mM), 1 μL DTT (100 mM), 2 μL 2′ OMTase (or water), 2 μL of FCE (or water) and 2 μL of T7 RNA polymerase (in its soluble, immobilized or fusion-protein form). All the reactions were performed with non-diluted (starting from a stock of 1 mg/ml T7 RNAP, 0.5 mg/ml FCE, 0.5 mg/ml 2′ OMTase and 0.5 mg/ml FCE-T7 RNAP) and 1:4 and 1:16 protein dilutions. All reactions were performed at 37, 40 and 45° C. for 1 h. After the reaction, 1 μL Turbo DNase I (2 U/μL, Invitrogen) was added to each sample, which were incubated 30 min more at 37° C. RNA was purified by using #NEB T2050L Monarch kit. The RNA yields of the obtained transcript (173 nucleotides) were measured by Nanodrop and compared in FIG. 58.

As shown in FIG. 58, soluble T7 RNAP, as tested, worked slightly better at 40° C. in presence of FCE. Co-immobilization in non-PEG750 coated magnetic beads resulted in a loss of around 50% of T7 RNAP activity, and displayed a similar rate at 37 and 40° C., while a notable drop at 45° C. (around 50%). Fusion FCE-T7 RNAP also shows a slightly better transcription efficiency at 40° C. but turns to be around 10% of the single soluble T7 RNAP activity.

### Example 38: LCMS Cap Incorporation Assay

Protection of a portion of an RNA substrate from the action of a single-stranded nucleotide-specific endoribonuclease (e.g., hRNase4) by hybridization with a complementary affinity tagged DNA probe (e.g., shorter than the RNA substrate) can be used to selectively isolate and analyze features of the protected portion, such as a cap structure and/or any modifications that are present. In some embodiments, methods may include contacting an RNA substrate with multiple probes (e.g., multiple biotinylated-DNA probes) targeting different portions of the RNA substrate, permitting simultaneous analysis of such portions. Disclosed methods may be applied to RNA modification analysis, such as RNA identification, locating an RNA within a sequence, assessing RNA stoichiometry, detecting RNA presence, permanence, and/or dynamics (i.e., installation and removal), and detecting co-existence of RNA modifications. For example, a method for measuring capping activity may include annealing a capped RNA substrate and aa DNA probe (e.g., a biotinylated DNA probe) which is complementary to at least a portion of the capped RNA substrate (e.g., a segment of interest) to form an RNA/DNA duplex. The duplex and an enzyme composition (e.g., an enzyme composition comprising hRNase4 and optionally an RNA end repair enzyme) may be combined to form a cleaved DNA-RNA hybrid duplex and one or more single-stranded RNA fragments of the RNA substrate. The cleaved DNA-RNA hybrid duplex may then be affinity purified (e.g., using streptavidin magnetic beads). The remaining portion of the RNA substrate included in the purified DNA-RNA hybrid duplex may be eluted, for example, by contacting the purified DNA-RNA hybrid duplex with a DNase I. In this example, a DNA probe sequence comprising desthiobiotin (e.g., CTTCTTTTCTCTCTTATTTCCC/3deSBioTEG/(SEQ ID NO: 26)) was hybridized to a co-transcriptionally capped IVT product using a touchdown hybridization approach (heating to 95° C. for 2 minutes, followed by slowly cooling to 22° C. at 0.1° C./s) in 1×NEBuffer 1 supplemented with 3 M urea. The hybridized mRNA solution was diluted to 1 M urea in NEBuffer 1 and a composition of hRNase4/T4 PNK was added. The mixture was incubated at 37° C. for 1.5 hours. Digestion was stopped by addition of human placental RNase inhibitor. Next, the resulting duplex comprising the 5′-biotinylated DNA probe and the corresponding hybridized RNA oligonucleotide was purified utilizing streptavidin magnetic beads. The hybridized RNA was eluted by incubation with DNase I at 37° C. The isolated RNA oligonucleotide was characterized by or LC-MS or LC-MS/MS. Comparative experiments were performed in the absence of either the DNA probe or hRNase4/T4 PNK. Example target RNase4 cleavage fragment include

(SEQ ID NO: 27)
GGGAAAUAAGAGAGAAAAGAAGAGU,
(SEQ ID NO: 28)
GGGAAAUAAGAGAGAAAAGAAGAG,
and
(SEQ ID NO: 29)
GGGAAAUAAGAGAGAAAAGAAGAGUA.

### Example 39: Lcms Analysis of Co-Transcriptional Capping

Co-transcriptional capping reactions were performed at 45° C. with linear pRNA21 DNA template as described in EXAMPLE 37, by adding 1 μM of each enzyme in the following reactions (24 of enzyme solution in 20 μL of reaction volume): soluble SNAP-T7 RNAP and SNAP-FCE (T7+FCE); immobilized SNAP-T7 RNAP and SNAP-FCE in BG-magnetic beads (T7+FCE@BG) and in BG and PEG750-magnetic beads (T7+FCE@PEG); soluble SNAP-FCE-T7 RNAP (FCE-T7); immobilized SNAP-FCE-T7 RNAP in BG-magnetic beads (FCE-T7@BG) and in BG and PEG750-magnetic beads (FCE-T7@PEG); soluble SNAP-T7 RNAP, SNAP-FCE and SNAP-2′-O-Methyltransferase (T7+FCE+2′ OMTase); immobilized SNAP-T7 RNAP, SNAP-FCE and SNAP-2′-O-Methyltransferase in BG-magnetic beads (T7+FCE+2′OMTase@BG) and in BG and PEG750-magnetic beads (T7+FCE+2′ OMTase@PEG); soluble SNAP-FCE-T7 RNAP and 2′-O-Methyltransferase (FCE-T7+2′OMTase); immobilized SNAP-FCE-T7 RNAP and 2′-O-Methyltransferase in BG-magnetic beads (FCE-T7+2′OMTase@BG) and in BG and PEG750-magnetic beads (FCE-T7+2′ OMTase@PEG).

After RNA purification, RNA yield was measured with Nanodrop. Each sample was prepared for LCMS analysis in accordance with EXAMPLE 38 by mixing 2.5 μL of H₂O, 6 μL of TRIS pH 7.0 (100 mM), 50 μL of each sample (2 μM) and 1.5 μL of RNase4 probe (100 μM; SEQ ID NO:26). The mixture was incubated at 80° C. for 0.5 min and ramp down at 0.1° C./s to 25° C. After the temperature ramp, the following mixture was added to the sample: 8 μL of 10× Buffer r1.1 (#NEB B7030S), 0.8 μL of RNase4 (1.13 μM), 1.2 μL of T4 polynucleotide kinase (10 U/μL) and 10 μL of H₂O. The mixture was incubated at 37° C. for 1 h. After that, 4 μL of human RNase inhibitor were added, and the mixture was incubated 10 min at room temperature. On the other hand, 80 μL of Streptavidin magnetic beads (#NEB S1420S) were washed two times with 100 μL of Buffer B (100 mM Tris pH 7.5, 10 mM EDTA, 50 mM NaCl) and resuspended in 50 μL of Buffer A (100 mM Tris pH 7.5, 10 mM EDTA, 500 mM NaCl). Buffer A was removed from beads, and the digestion was added, incubating 15 min at room temperature with agitation. The supernatant was removed, and beads were washed again one time with Buffer B, two times with Buffer A and one last time with Buffer B (5 min at room temperature with shaking). RNA was eluted from beads by heating at 65° C. for 5 min adding 35 μL of water. The eluant was filtered through Millipore 0.2 mm spin filter (16,000 rcf, room temperature, 5 min). The sample was sent to LC-MS analysis.

As shown in FIG. 59A, all reactions with T7 RNAP and FCE yielded RNA products comprising RNA having Cap 0. T7 RNAP and FCE co-immobilization and FCE-T7 fusion protein seem to be a successful strategy to obtain Cap 0 product, achieving the maximum rate (>90%) in soluble FCE-T7 RNAP reaction. Capping efficiency was higher compared with individual soluble T7 RNAP and FCE reaction (around 60%), as catalytic T7 RNAP and FCE need to be closer in space to assess co-transcriptional capping properly. Similarly, all reactions with T7 RNAP, FCE and 2′-O-Methyltransferase yielded RNA products comprising RNA having Cap 1, achieving in all cases around 50% of the final product, and showing that co-immobilization of both T7 RNAP, FCE and FCE-T7 RNAP with 2′-O-Methyltransferase does not suggest a relevant activity loss of any of the enzymes.

What is claimed is:

1. An immobilized enzyme comprising:
an enzyme selected from a type IIS restriction endonuclease, an RNA polymerase, and a capping enzyme,
a peptide linker attached to the enzyme by a peptide bond,
a SNAP-tag attached to the linker by a peptide bond,
O⁶-benzyleguanine bound to the SNAP-tag; and
magnetic beads having a surface modification comprising the O⁶-benzyleguanine.

2. An immobilized enzyme according to claim 1, wherein the type IIS restriction endonuclease has a recognition sequence and cleave site of 5′ GCTCTTC N1 3′ or 5′ GCTCTTC N1/N4 3′ or the RNA polymerase is T7 RNA polymerase or the capping enzyme is Faustovirus capping enzyme or 2′O-methyltransferase.

3. An immobilized BspQI comprising:
BspQI,
a glycine-serine linker attached to the BspQI by a peptide bond,
a SNAP-tag attached to the linker by a peptide bond,
O⁶-benzyleguanine bound to the SNAP-tag; and
magnetic beads having a surface modification comprising the O⁶-benzyleguanine.

4. An immobilized T7 RNA polymerase comprising:
T7 RNA polymerase,
a glycine-serine linker attached to the T7 RNA polymerase by a peptide bond,
a SNAP-tag attached to the linker by a peptide bond,
O⁶-benzyleguanine bound to the SNAP-tag; and
magnetic beads having a surface modification comprising the O⁶-benzyleguanine.

5. An immobilized Faustovirus capping enzyme comprising:
Faustovirus capping enzyme,
a glycine-serine linker attached to the Faustovirus capping enzyme by a peptide bond,
a SNAP-tag attached to the linker by a peptide bond,
O⁶-benzyleguanine bound to the SNAP-tag; and
magnetic beads having a surface modification comprising the O⁶-benzyleguanine.

6. A method of cleaving a double stranded DNA substrate, the method comprising:
contacting a first portion of the double stranded DNA substrate with an immobilized enzyme comprising a type IIS restriction endonuclease to produce double stranded DNA cleavage products;
separating the immobilized enzyme from the double stranded DNA cleavage products to form separated immobilized enzyme and separated double stranded DNA cleavage products; and
contacting a second portion of the double stranded DNA substrate with the separated immobilized enzyme comprising a type IIS restriction endonuclease to produce more double stranded DNA cleavage products.

7. A method according to claim 6, further comprising repeating the separating and subsequent contacting steps from 2 to 50 times.

8. A method according to claim 6, further comprising combining the separated double stranded DNA cleavage products with the more double stranded DNA cleavage products to produce pooled products.

9. A method according to claim 6, wherein the enzyme is BspQI or Nt.BspQI.

10. A method of cleaving a double stranded DNA substrate, the method comprising:
contacting the double stranded DNA substrate with an immobilized enzyme comprising a type IIS restriction endonuclease to produce double stranded DNA cleavage products, wherein the double stranded DNA cleavage products comprise at least one nick;
separating the immobilized enzyme from the double stranded DNA cleavage products to form separated immobilized enzyme and separated double stranded DNA cleavage products; and
contacting the separated double stranded DNA cleavage products with a second enzyme.

11. A method according to claim 10, wherein the double stranded DNA substrate comprises, in a 5′-3′ direction, a coding sequence, a poly(U) sequence, and a type IIS restriction endonuclease recognition site.

12. A method according to claim 11, wherein the type IIS restriction endonuclease is BspQI and the type IIS restriction endonuclease recognition site is a BspQI recognition site.

13. A method of producing RNA comprising:
(a) contacting a polynucleotide template with an immobilized enzyme to form a cleaved polynucleotide template; and
(b) contacting the cleaved polynucleotide template with an RNA polymerase to produce transcription products comprising the RNA,
wherein the polynucleotide template comprises, in a 5′-3′ direction, a coding sequence, a poly(U) sequence, and a type IIS restriction endonuclease recognition site and the immobilized enzyme comprises a type IIS restriction endonuclease, a support, and a linker disposed between the type IIS restriction endonuclease and the support.

14. A method according to claim 13, wherein the (a) contacting and the (b) contacting are performed as a coupled reaction.

15. A method according to claim 13, wherein the RNA comprises, in a 5′-3′ direction, a sequence complementary to the coding sequence and a poly(A) sequence.

16. A method according to claim 13, wherein the type IIS restriction endonuclease is BspQI and the type IIS restriction endonuclease recognition site is a BspQI recognition site.

17. A method according to claim 13, wherein the RNA polymerase is a type II RNA polymerase.

18. A method according to claim 13, further comprising contacting the RNA with a capping enzyme to form a capped RNA product.

19. A method according to claim 13, further comprising chemically capping the RNA to form a capped RNA product.

20. An in vitro transcription method, the method comprising:
(a) contacting a double stranded DNA template with a first enzyme to form a nicked template, the first enzyme comprising a type IIS restriction endonuclease;
(b) contacting the nicked template with a second enzyme to form a transcription product, the second enzyme comprising an RNA polymerase; and
(c) optionally contacting the transcription product with a third enzyme to form a capped transcription product, the third enzyme comprising a capping enzyme, wherein at least one of the first enzyme, the second enzyme, and the third enzyme are immobilized enzymes.

21. A method according to claim 20, wherein the type IIS restriction endonuclease is BspQI or Nt.BspQI.

22. A method according to claim 20, wherein the RNA polymerase is T7 RNA polymerase.

23. A method according to claim 20, wherein the capping enzyme is Faustovirus capping enzyme.

24. A method according to claim 20, wherein the contacting the transcription product with the capping enzyme further comprises contacting the transcription product with a Cap-0 capping enzyme to form a Cap-0 transcription product and optionally contacting the Cap-0 transcription product with a Cap-1 capping enzyme to form a Cap-1 transcription product.

25. A method according to claim 22, wherein the Cap-0 enzyme is Faustovirus capping enzyme.

26. A method according to claim 22, wherein the Cap-1 enzyme is 2′O-methyltransferase.

27. A method according to claim 22, wherein the capping efficiency is at least 40%.

28. An in vitro transcription system, the system comprising a first reactor having an inlet, an outlet, and a reaction chamber disposed between the inlet and the outlet, wherein the first reaction chamber holds one or more enzymes each independently selected from a soluble enzyme, a soluble fusion protein comprising one or more enzymes, an immobilized enzyme, and an immobilized fusion protein comprising one or more enzymes, and wherein the first reactor comprises:
a soluble type IIS restriction endonuclease,
a soluble RNA polymerase,
a soluble capping enzyme,
a soluble fusion protein comprising an RNA polymerase and a capping enzyme,
an immobilized type IIS restriction endonuclease
an immobilized RNA polymerase,
an immobilized capping enzyme,
an immobilized fusion protein comprising an RNA polymerase and a capping enzyme, or
combinations thereof,
optionally wherein the immobilized type IIS restriction endonuclease, the immobilized RNA polymerase, or the immobilized capping enzyme is co-immobilized on a support with one or both of the other two enzymes.
